(19) United States
(12) Patent Application Publication
(10) Pub. No.: US 2021/0403997 A1
(43) Pub. Date: Dec. 30, 2021

(54) DEVICE, NUCLEIC ACID TESTING METHOD AND NUCLEIC ACID TESTING DEVICE, AND GENE TESTING METHOD

(71) Applicants: Ricoh Company, Ltd., Tokyo (JP); FASMAC CO., LTD., Atsugi-shi (JP)

(72) Inventors: Michie HASHIMOTO, Tokyo (JP); Hirotaka UNNO, Kanagawa (JP); Satoshi NAKAZAWA, Kanagawa (JP); Yuki YONEKAWA, Kanagawa (JP); Satoshi FUTO, Kanagawa (JP); RIZTYAN, Kanagawa (JP)

(73) Assignees: Ricoh Company, Ltd., Tokyo (JP); FASMAC CO., LTD., Atsugi-shi (JP)

(21) Appl. No.: 17/414,755

(22) PCT Filed: Dec. 16, 2019
(86) PCT No.: PCT/JP2019/049088

§ 371 Date: Jun. 16, 2021

(30) Foreign Application Priority Data
Dec. 18, 2018 (JP) 2018-236805
Dec. 4, 2019 (JP) 2019-219787

(51) Int. Cl. C12Q 1/6876; C12Q 1/686; B01L 3/00

(52) U.S. Cl. CPC C12Q 1/6876; B01L 2200/0689; B01L 3/50853; C12Q 1/686

(57) ABSTRACT

Provided is a device including a well provided in a number of at least one, wherein a nucleic acid having at least one of a full-length nucleotide sequence and a partial nucleotide sequence of rRNA or rDNA is contained in a defined copy number in at least one well of the well, and wherein the defined copy number of the nucleic acid having at least one of a full-length nucleotide sequence and a partial nucleotide sequence of rRNA or rDNA is 1,000 or less.

### TECHNICAL FIELD

The present disclosure relates to a device, a nucleic acid testing method and a nucleic acid testing device, and a gene testing method.

### BACKGROUND ART

Purposes of gene testing include examining nuclear genomes and detecting rRNA and rDNA. An rDNA gene (rDNA) codes for rRNA, and rRNA constitutes a ribosome. Bacteria have 23S rRNA, 16S rRNA, and 5S rRNA depending on the size of the bacteria. Eucaryotes have 28S rRNA, 18S rRNA, 5.8S rRNA, and 5S rRNA. Because rRNAs have a high sequence conservability, rRNAs can be used for detecting a wide variety of species. On the other hand, because different species have different mutated points, rRNAs are also used for species, breeds, and lineages identification. These methods can be used for, for example, specific detection of pork and species identification of eels.

As a method for detecting rRNA or rDNA, there has been a proposed testing method such as PCR or real-time PCR including design of primer on sequence that can be used for species identification (for example, see PTL 1).

There has also been a proposed method of distinguishing a false-negative determination based on use of a standard molecule for real-time PCR testing including both of: an artificial DNA sequence of the target region of rRNA or rDNA that can be amplified under the same PCR conditions in the same reaction tube; and the sequence of the testing target DNA (for example, see PTL 2).

### CITATION LIST

### Patent Literature

PTL 1: Japanese Translation of PCT International Application Publication No. JPT-2010-530763

PTL 2: International Publication No. WO 2009/157465

### SUMMARY OF INVENTION

### Technical Problem

The present disclosure has an object to provide a device that can detect a nucleic acid contained in a sample and having at least one of a full-length nucleotide sequences and a partial nucleotide sequence of rRNA or rDNA, can avoid a false-negative determination more infallibly and enable an accurate qualitative testing including positive or negative detection particularly when the copy number of the nucleic acid having at least one of a full-length nucleotide sequence and a partial nucleotide sequence of rRNA or rDNA is low, and can measure the copy number more accurately in quantitative PCR.

### Solution to Problem

According to one aspect of the present disclosure, a device includes a well provided in a number of at least one copy. A nucleic acid having at least one of a full-length nucleotide sequence and a partial nucleotide sequence of rRNA or rDNA is contained in a defined copy number in at least one well. The defined copy number of the nucleic acid having at least one of a full-length nucleotide sequence and a partial nucleotide sequence of rRNA or rDNA is 1,000 or less.

### Advantageous Effects of Invention

The present disclosure can provide a device that can detect a nucleic acid contained in a sample and having at least one of a full-length nucleotide sequence and a partial nucleotide sequence of rRNA or rDNA and can avoid a false-negative determination more infallibly and enable an accurate qualitative testing including positive or negative detection particularly when the copy number of the nucleic acid having at least one of a full-length nucleotide sequence and a partial nucleotide sequence of rRNA or rDNA is low. The present disclosure can also provide a device that enables an accurate quantitative testing of a nucleic acid contained in a sample and having at least one of a full-length nucleotide sequence and a partial nucleotide sequence of rRNA or rDNA.

### BRIEF DESCRIPTION OF DRAWINGS

FIG. 1 is a perspective view illustrating an example of a device of the present disclosure.

FIG. 2 is a perspective view illustrating another example of a device of the present disclosure.

FIG. 3 is a side view of FIG. 2.

FIG. 4 is a perspective view illustrating another example of a device of the present disclosure.

FIG. 5 is a view illustrating an example of positions of wells in a device of the present disclosure to be filled with a nucleic acid having at least one of a full-length nucleotide sequence and a partial nucleotide sequence of rRNA or rDNA in a defined copy number.

FIG. 6 is a view illustrating another example of positions of wells in a device of the present disclosure to be filled with a nucleic acid having at least one of a full-length nucleotide sequence and a partial nucleotide sequence of rRNA or rDNA in a defined copy number.

FIG. 7 is a graph plotting a relationship between a copy number having variation according to a Poisson distribution and a coefficient of variation CV.

FIG. 8 is a graph plotting an example of a relationship between the frequency and the fluorescence intensity of cells in which DNA replication has occurred.

FIG. 9A is an exemplary diagram illustrating an example of an electromagnetic valve-type discharging head.

FIG. 9B is an exemplary diagram illustrating an example of a piezo-type discharging head.

FIG. 9C is an exemplary diagram illustrating a modified example of the piezo-type discharging head illustrated in FIG. 9B.

FIG. 10A is an exemplary graph plotting an example of a voltage applied to a piezoelectric element.

FIG. 10B is an exemplary graph plotting another example of a voltage applied to a piezoelectric element.

FIG. 11A is an exemplary diagram illustrating an example of a liquid droplet state.

FIG. 11B is an exemplary diagram illustrating an example of a liquid droplet state.

FIG. 11C is an exemplary diagram illustrating an example of a liquid droplet state.

FIG. 12 is a schematic diagram illustrating an example of a dispensing device configured to land liquid droplets sequentially into wells.

FIG. 13 is an exemplary diagram illustrating an example of a liquid droplet forming device.

FIG. 14 is a diagram illustrating hardware blocks of a control unit of the liquid droplet forming device of FIG. 13.

FIG. 15 is a diagram illustrating functional blocks of a control unit of the liquid droplet forming device of FIG. 14.

FIG. 16 is a flowchart illustrating an example of an operation of a liquid droplet forming device.

FIG. 17 is an exemplary diagram illustrating a modified example of a liquid droplet forming device.

FIG. 18 is an exemplary diagram illustrating another modified example of a liquid droplet forming device.

FIG. 19A is a diagram illustrating a case where two fluorescent particles are contained in a flying liquid droplet.

FIG. 19B is a diagram illustrating a case where two fluorescent particles are contained in a flying liquid droplet.

FIG. 20 is a graph plotting an example of a relationship between a luminance Li when particles do not overlap each other and a luminance Le actually measured.

FIG. 21 is an exemplary diagram illustrating another modified example of a liquid droplet forming device.

FIG. 22 is an exemplary diagram illustrating another example of a liquid droplet forming device.

FIG. 23 is an exemplary diagram illustrating an example of a method for counting cells that have passed through a micro-flow path.

FIG. 24 is an exemplary diagram illustrating an example of a method for capturing an image of a portion near a nozzle portion of a discharging head.

FIG. 25 is a graph plotting a relationship between a probability P (>2) and an average cell number.

FIG. 26 is a block diagram illustrating an example of a hardware configuration of a nucleic acid testing device.

FIG. 27 is a diagram illustrating an example of a functional configuration of a nucleic acid testing device.

FIG. 28 is a flowchart illustrating an example of procedures of a program for a nucleic acid testing device;

FIG. 29 is a diagram illustrating an example of nucleic acid sample positioning in Example of the present disclosure.

FIG. 30 is a diagram illustrating the results of quantitative PCR in Example of the present disclosure.

FIG. 31 is a graph plotting the results of quantitative PCR in Example of the present disclosure.

FIG. 32 is a graph plotting the results of quantitative PCR in Example of the present disclosure.

### DESCRIPTION OF EMBODIMENTS

(Device)

A device of the present disclosure includes a well provided in the number of at least one. A nucleic acid having at least one of a full-length nucleotide sequence and a partial nucleotide sequence of rRNA or rDNA is contained in a defined copy number in at least one well. The defined copy number of the nucleic acid having at least one of a full-length nucleotide sequence and a partial nucleotide sequence of rRNA or rDNA is 1,000 or less. The device includes an identifier unit, and further includes other components as needed.

The present disclosure is based on the following finding. With existing devices containing a reference nucleic acid in a well in an unspecified copy number, a result of amplification of the reference nucleic acid obtained when the reference nucleic acid is allowed to undergo an amplification reaction has a low reliability.

In the device of the present disclosure, a reference nucleic acid, i.e., a nucleic acid having at least one of a full-length nucleotide sequence and a partial nucleotide sequence of rRNA or rDNA is located in each well in a defined copy number with a filling accuracy of a certain level or higher (with a coefficient of variation of a certain level or lower).

The device of the present disclosure can avoid a false-negative determination more infallibly and can be used for qualitative testing with an improved negative determination accuracy, because the copy number of a reference nucleic acid contained in a well, i.e., a nucleic acid having at least one of a full-length nucleotide sequence and a partial nucleotide sequence of rRNA or rDNA is a defined copy number. That is, the device of the present disclosure can be used for an accurate qualitative testing including positive or negative detection. The device of the present disclosure can also be used for an accurate quantitative testing of rRNA or rDNA contained in a sample, because the copy number of a reference nucleic acid contained in a well, i.e., a nucleic acid having at least one of a full-length nucleotide sequence and a partial nucleotide sequence of rRNA or rDNA is a defined copy number.

The device of the present disclosure will be described below.

In the present specification, a device in which a nucleic acid having at least one of a full-length nucleotide sequence and a partial nucleotide sequence of rRNA or rDNA is contained in a defined copy number is referred to as “device”. A device in which a nucleic acid having at least one of a full-length nucleotide sequence and a partial nucleotide sequence of rRNA or rDNA is not contained in a defined copy number may be referred to as “plate”.

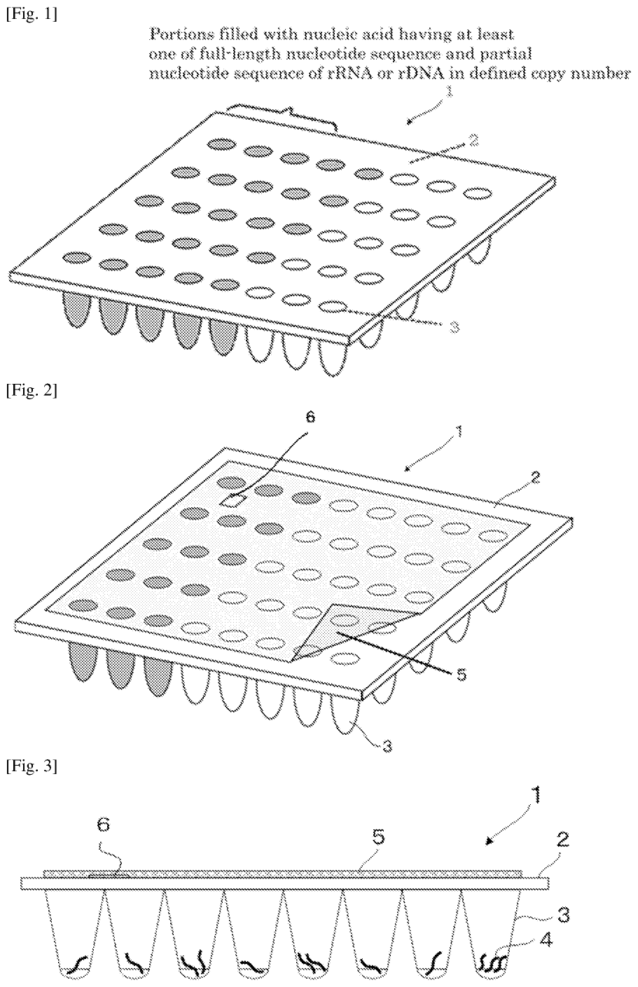

FIG. 1 is a perspective view illustrating an example of the device of the present disclosure. FIG. 2 is a perspective view illustrating another example of the device of the present disclosure. FIG. 3 is a side view of the device of FIG. 2.

In the view, the device 1 includes a base material 2 provided with a plurality of wells 3. The wells include wells to be filled with a nucleic acid 4 having at least one of a full-length nucleotide sequence and a partial nucleotide sequence of rRNA or rDNA in a defined copy number. Other wells (empty wells in the view) are to be filled with a sample. As described below, when the device of the present disclosure is configured to be filled with an amplifiable reagent different from a testing target sample (i.e., a sample that may possibly contain rRNA or rDNA), the amplifiable reagent may be filled in a well in which the testing target sample is located. In FIG. 2 and FIG. 3, the reference numeral 5 denotes a sealing member. Further, as illustrated in FIG. 2 and FIG. 3, the device 1 may include an IC chip or a barcode (identifier unit 6) storing information on the number of the reagent filled in each well 3 and the uncertainty (or certainty) of the number, or information related with these kinds of information at a position that is between the sealing member 5 and the base material 2 and does not overlap the openings of the wells. This is suitable for preventing, for example, unintentional alteration of the identifier unit.

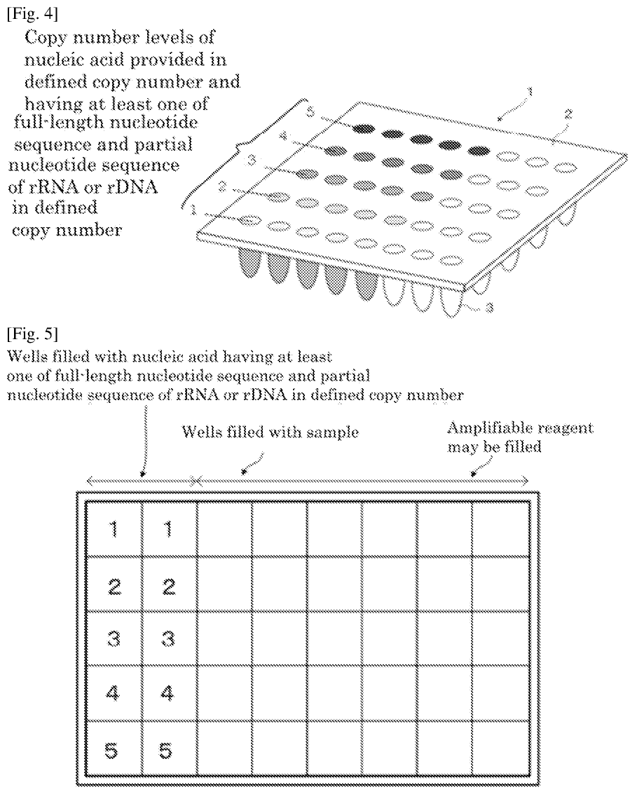

With the identifier unit, the device can be distinguished from a common well plate that does not have an identifier unit. Therefore, confusion or mistake can be prevented. FIG. 4 is a perspective view illustrating another example of the device of the present disclosure. In the device of FIG. 4, levels of the copy number of the nucleic acid having at least one of a full-length nucleotide sequence and a partial nucleotide sequence of rRNA or rDNA include the following five levels: 1, 2, 3, 4, and 5.

FIG. 5 is a view illustrating an example of the positions of wells in the device of the present disclosure to be filled with the nucleic acid having at least one of a full-length nucleotide sequence and a partial nucleotide sequence of rRNA or rDNA in a defined copy number. The numerals in the wells in FIG. 5 indicate specific numbers as the defined copy number of the nucleic acid having at least one of a full-length nucleotide sequence and a partial nucleotide sequence of rRNA or rDNA. Wells in which no numerals are indicated in FIG. 5 are to be filled with the testing target sample. Further, the wells in which no numerals are indicated in FIG. 5 may be filled with an amplifiable reagent in addition to the testing target sample.

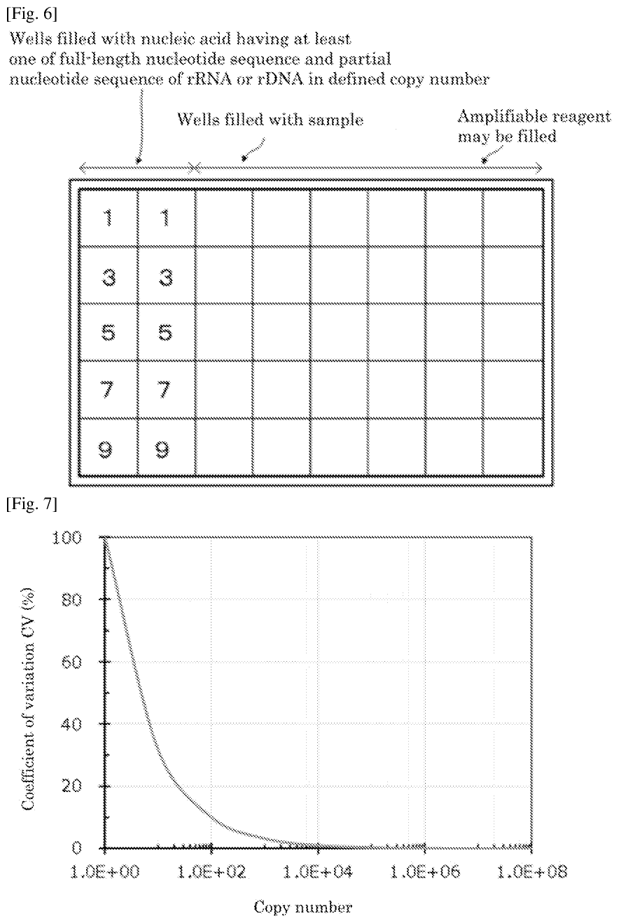

FIG. 6 is a view illustrating another example of the positions of wells in the device of the present disclosure to be filled with the nucleic acid having at least one of a full-length nucleotide sequence and a partial nucleotide sequence of rRNA or rDNA in a defined copy number. The numerals in the wells in FIG. 6 indicate specific numbers as the defined copy number of the nucleic acid having at least one of a full-length nucleotide sequence and a partial nucleotide sequence of rRNA or rDNA. Wells in which no numerals are indicated in FIG. 6 are to be filled with the testing target sample. Further, the wells in which no numerals are indicated in FIG. 6 may be filled with an amplifiable reagent in addition to the testing target sample.

In the present disclosure, a copy number means the number in which the nucleotide sequence (target nucleotide sequence) of the nucleic acid having at least one of a full-length nucleotide sequence and a partial nucleotide sequence of rRNA or rDNA is contained in the well.

The target nucleotide sequence refers to a nucleotide sequence including defined nucleotide sequences in at least primer and probe regions. Particularly, a nucleotide sequence having a defined total length is also referred to as specific nucleotide sequence.

In the present disclosure, a defined copy number refers to the aforementioned copy number that specifies the number of target nucleotide sequences at accuracy of a certain level or higher.

This means that the defined copy number is known as the number of target nucleotide sequences actually contained in a well. That is, the defined copy number in the present disclosure is more accurate or reliable as a number than a predetermined copy number (calculated estimated value) obtained according to existing serial dilution methods, and is a controlled value that has no dependency on a Poisson distribution even if the value is within a low copy number region of 1,000 or lower in particular. When it is said that the defined copy number is a controlled value, it is applicable that a coefficient of variation CV expressing uncertainty roughly satisfy either CV<1/√x with respect to an average copy number x or CV≥20%. Hence, use of a device including wells in which a target nucleotide sequence is contained in the defined copy number makes it possible to perform qualitative or quantitative testing of a sample containing the target nucleotide sequence more accurately than ever.

When the number of target nucleotide sequences and the number of nucleic acid molecules including the sequence coincide with each other, “copy number” and “number of molecules” may be associated with each other.

Specifically, for example, in the case of norovirus, when the number of viruses is 1, the number of nucleic acid molecules is 1 and the copy number is 1. In the case of yeast at a GI phase, when the number of yeast cells is 1, the number of nucleic acid molecules (the number of same chromosomes) is 1 and the copy number is 1. In the case of human cell at a G0/GI phase, when the number of human cells is 1, the number of nucleic acid molecules (the number of same chromosomes) is 2 and the copy number is 2.

Further, in the case of yeast at a GI phase having the target nucleotide sequence introduced at two positions, when the number of yeast cells is 1, the number of nucleic acid molecules (the number of same chromosomes) is 1 and the copy number is 2. In the present disclosure, a defined copy number of the nucleic acid having at least one of a full-length nucleotide sequence and a partial nucleotide sequence of rRNA or rDNA may be referred to as predetermined number or absolute number of the nucleic acid having at least one of a full-length nucleotide sequence and a partial nucleotide sequence of rRNA or rDNA.

As the defined copy number, it is applicable to provide two or more different integers.

Examples of a combination of the defined copy number (specific number) include a combination of 1, 2, 3, 4, 5, 6, 7, 8, 9, and 10, a combination of 1, 3, 5, 7, and 9, and a combination of 2, 4, 6, 8, and 10.

A combination of the defined copy number (specific number) may be, for example, a combination of the following four levels: 1, 10, 100, and 1,000.

Use of the results of amplification of a plurality of nucleic acids provided in different defined copy numbers and having at least one of a full-length nucleotide sequence and a partial nucleotide sequence of rRNA or rDNA makes it possible to generate a calibration curve. Use of the calibration curve makes it possible to perform an accurate quantification of nucleic acid such as rRNA or rDNA contained in the testing target sample.

<Nucleic Acid Having at Least any One of Full-Length Nucleotide Sequence and Partial Nucleotide Sequence of rRNA or rDNA>

The nucleic acid having at least one of a full-length nucleotide sequence and a partial nucleotide sequence of rRNA or rDNA is rRNA or rDNA of a cell.

The rRNA refers to a ribosomal RNA.

Examples of the rRNA of bacteria include 23S rRNA, 16S rRNA, and 5S rRNA depending on the size. Examples of the rRNA of eukaryotes include 28S rRNA, 18S rRNA, 5.8S rRNA, and 5S rRNA depending on the size.

The 12S rRNA is RNA of 12S subunit, which is one of subunits of ribosomes, which are organelles.

The rDNA is a ribosomal RNA gene.

The rDNA is DNA coding the rRNA.

In the present specification, “at least one of a full-length nucleotide sequence and a partial nucleotide sequence of rRNA or rDNA” means the following combination patterns.

(1-1) a full-length nucleotide sequence of rRNA

(1-2) a partial nucleotide sequence of rRNA

(1-3) both of a full-length nucleotide sequence of rRNA and a partial nucleotide sequence of rRNA

(2-1) a full-length nucleotide sequence of rDNA

(2-2) a partial nucleotide sequence of rDNA

(2-3) both of a full-length nucleotide sequence of rDNA and a partial nucleotide sequence of rDNA

The nucleic acid having at least one of a full-length nucleotide sequence and a partial nucleotide sequence of rRNA or rDNA is not particularly limited. Examples of the nucleic acid having at least one of a full-length nucleotide sequence and a partial nucleotide sequence of rRNA or rDNA include a nucleic acid into which a nucleotide sequence of 12S rRNA extracted from a cell harvested from a pig tissue is introduced, a nucleic acid into which an artificially synthesized nucleotide sequence of 12S rRNA is introduced, a nucleotide sequence of 16S rDNA extracted from a cell harvested from an eel tissue, a nucleic acid into which an artificially synthesized nucleotide sequence of 16S rRNA is introduced, and 16S rRNA of various bacteria.

The nucleic acid having at least one of a full-length nucleotide sequence and a partial nucleotide sequence of rRNA or rDNA includes a positive single strand RNA. The nucleic acid having at least one of a full-length nucleotide sequence and a partial nucleotide sequence of rRNA or rDNA may be modified or mutated.

The nucleic acid having at least one of a full-length nucleotide sequence and a partial nucleotide sequence of rRNA or rDNA may be in a bared state in a well or may be carried in a carrier in a well. The state of being carried in a carrier is preferable. The carrier is not particularly limited and may be appropriately selected depending on the intended purpose as long as the carrier can carry a nucleic acid. Examples of the carrier include cells, liposomes, microcapsules, phages, and viruses. Among these carriers, cells are preferable.

After parts of nucleotide sequences extracted from tissues are introduced by transgenesis as RNA and DNA into nucleic acids intrinsic to the cells, the number by which the nucleotide sequence of the nucleic acid having at least one of a full-length nucleotide sequence and a partial nucleotide sequence of rRNA or rDNA is present can be obtained by measuring the number of carriers into which RNA and DNA have been introduced by transgenesis, since one nucleic acid (one copy) is present per carrier.

The nucleotide sequence of the nucleic acid having at least one of a full-length nucleotide sequence and a partial nucleotide sequence of rRNA or rDNA is not particularly limited and may be appropriately selected depending on the intended purpose. For example, when detection of a pig or an eel or species identification is the intended purpose, examples of the nucleotide sequence of the nucleic acid having at least one of a full-length nucleotide sequence and a partial nucleotide sequence of rRNA or rDNA include pig 12S rRNA or rDNA, and eel 16S rRNA or rDNA.

The pig is not particularly limited and may be appropriately selected depending on the intended purpose.

The eel is not particularly limited and may be appropriately selected depending on the intended purpose. Examples of the eel include Japanese eel.

Examples of the nucleotide sequence of the pig 12S rDNA include SEQ ID NO. 1.

Examples of the nucleotide sequence of the eel 16S rDNA include SEQ ID NO. 5.

It is applicable that the total length of the nucleic acid having at least one of a full-length nucleotide sequence and a partial nucleotide sequence of rRNA or rDNA be 50 nucleotides or more.

It is applicable that the nucleic acid having at least one of a full-length nucleotide sequence and a partial nucleotide sequence of rRNA or rDNA have a nucleotide sequence having a homology of 80% or higher with respect to the nucleotide sequence of SEQ ID NO. 1 or a nucleotide sequence having an arbitrary length.

Or, it is applicable that the nucleic acid having at least one of a full-length nucleotide sequence and a partial nucleotide sequence of rRNA or rDNA have a nucleotide sequence having a homology of 80% or higher with respect to the nucleotide sequence of SEQ ID NO. 5 or a nucleotide sequence having an arbitrary length.

It is applicable that the nucleic acid having at least one of a full-length nucleotide sequence and a partial nucleotide sequence of the pig 12S rRNA or rDNA include a nucleotide sequence X including: the nucleotide sequence of SEQ ID NO. 1; and a nucleotide sequence having an arbitrary length less than or equal to 1,000 nucleotides at a 5′ terminal side or a 3′ terminal side, and a nucleotide sequence Y having a homology of 80% or higher with respect to the nucleotide sequence X.

Or it is applicable that the nucleic acid having at least one of a full-length nucleotide sequence and a partial nucleotide sequence of the eel 16S rRNA or rDNA include a nucleotide sequence X including: the nucleotide sequence of SEQ ID NO. 5; and a nucleotide sequence having an arbitrary length less than or equal to 1,000 nucleotides at a 5′ terminal side or a 3′ terminal side, and a nucleotide sequence Y having a homology of 80% or higher with respect to the nucleotide sequence X.

The nucleotide sequence X including: the nucleotide sequence of SEQ ID NO. 1; and a nucleotide sequence having an arbitrary length less than or equal to 1,000 nucleotides at a 5′ terminal side or a 3′ terminal side is not particularly limited and may be appropriately selected depending on the intended purpose.

The nucleotide sequence Y having a homology of 80% or higher with respect to the nucleotide sequence X is not particularly limited and may be appropriately selected depending on the intended purpose.

The order of the nucleotide sequence X and the nucleotide sequence Y is not particularly limited and may be appropriately selected depending on the intended purpose. For example, from the 5′ terminal side, the nucleotide sequence X may be succeeded by the nucleotide sequence Y. Alternatively, from the 5′ terminal side, the nucleotide sequence Y may be succeeded by the nucleotide sequence X.

It is applicable that the well containing the nucleic acid having at least one of a full-length nucleotide sequence and a partial nucleotide sequence of pig 12S rRNA or rDNA contain at least any one of primers of SEQ ID NOS. 2 and 3, a probe of SEQ ID NO. 4, and an amplification reagent for a PCR reaction or contain at least any one of primers of SEQ ID NOS. 9, 10, 11, 12, 13, and 14 and an amplification reagent for a LAMP reaction.

Or it is applicable that the well containing the nucleic acid having at least one of a full-length nucleotide sequence and a partial nucleotide sequence of eel 16S rRNA or rDNA contain at least any one of primers of SEQ ID NOS. 6 and 7, a probe of SEQ ID NO. 8, and an amplification reagent for a PCR reaction or contain at least any one of primers of SEQ ID NOS. 15, 16, 17, 18, 19, and 20 and an amplification reagent for a LAMP reaction.

When the eel is Japanese eel, it is applicable that the well containing the nucleic acid having at least one of a full-length nucleotide sequence and a partial nucleotide sequence of Japanese eel 16S rRNA or rDNA contain at least any one of primers of SEQ ID NOS. 21 and 22, a probe of SEQ ID NO. 23, and an amplification reagent for a PCR reaction or contain at least any one of primers of SEQ ID NOS. 24, 25, 26, 27, 28, and 29 and an amplification reagent for a LAMP reaction.

LAMP is one of the gene amplification methods and the abbreviation for LoopMediated Isothermal Amplification. LAMP is characterized in that LAMP requires at least four kinds of primers utilizing six kinds of regions whereas PCR requires two kinds of primers. LAMP reaction proceeds at a constant temperature of around from 60 degrees C. to 65 degrees C. whereas PCR runs in three-step temperature changes i.e. denaturing, annealing, and extension. The LAMP is a gene amplification method that uses an enzyme having a 5′ →3′ DNA polymerase activity and a strand displacement activity and continuously induces DNA elongation in which a primer sequence serves as a template, to enable an explosive amplification reaction in a short time.

It is applicable to confirm that one copy (one molecule) of the nucleic acid having at least one of a full-length nucleotide sequence and a partial nucleotide sequence of rRNA or rDNA is introduced per cell by transgenesis. When the copy number of the nucleic acid having at least one of a full-length nucleotide sequence and a partial nucleotide sequence of rRNA or rDNA (i.e., a specific nucleotide sequence) coincides with the number of nucleic acid molecules including that sequence, “copy number” and “number of molecules” may be associated with each other.

The method for confirming that one copy (one molecule) of the nucleic acid having at least one of a full-length nucleotide sequence and a partial nucleotide sequence of rRNA or rDNA is introduced is not particularly limited and may be appropriately selected depending on the intended purpose. Examples of the method include DNA sequencing, a PCR method, and a Southern blotting method.

The number of kinds of the nucleic acid having at least one of a full-length nucleotide sequence and a partial nucleotide sequence of rRNA or rDNA to be introduced by transgenesis may be one, or two or more. Also in the case of introducing only one kind of a nucleic acid by transgenesis, nucleotide sequences of the same kind may be introduced in tandem depending on the intended purpose.

The method for transgenesis is not particularly limited and may be appropriately selected depending on the intended purpose as long as the method can introduce the nucleic acid having at least one of a full-length nucleotide sequence and a partial nucleotide sequence of rRNA or rDNA by an intended copy number at an intended position. Examples of the method include homologous recombination, CRISPR/Cas9, CRISPR/Cpf1, TALEN, Zinc finger nuclease, Flip-in, and Jump-in. When the carrier is a yeast fungus, homologous recombination is preferable among these methods in terms of a high transgenesis efficiency and ease of controlling.

Two or more of the wells in the device contain the nucleic acid having at least one of a full-length nucleotide sequence and a partial nucleotide sequence of rRNA or rDNA in the defined copy number. It is applicable that the defined copy number of the nucleic acid having at least one of a full-length nucleotide sequence and a partial nucleotide sequence of rRNA or rDNA in one of the wells be different from the defined copy number of the nucleic acid having at least one of a full-length nucleotide sequence and a partial nucleotide sequence of rRNA or rDNA in another of the wells.

The device of the present disclosure includes wells in which a testing target sample is to be located, in addition to the wells in which the nucleic acid having at least one of a full-length nucleotide sequence and a partial nucleotide sequence of rRNA or rDNA is located in the defined copy number. The wells in which the testing target sample is to be located may be filled with a predetermined amount of an amplifiable reagent different from the testing target sample. The predetermined amount needs at least to be a sufficiently detectable amount. When amplification of the amplifiable reagent has occurred, it can be confirmed that the amplification reaction is successful in the well in which the amplifiable reagent is located. Hence, the result of amplification of the testing target sample in the same well in which the amplifiable reagent is located is ensured in better reliability.

A nucleic acid is suitable for use as the amplifiable reagent. It is applicable that the nucleic acid be introduced in a nucleic acid of a cell.

The “nucleic acid” serving as the amplifiable reagent and the “cell” serving as a carrier, both used in the device of the present disclosure, will be described in detail below.

—Nucleic Acid—

The nucleic acid means a polymeric organic compound in which a nitrogencontaining base derived from purine or pyrimidine, sugar, and phosphoric acid are bonded with one another regularly. Examples of the nucleic acid also include a fragment of a nucleic acid or an analog of a nucleic acid or of a fragment of a nucleic acid.

The nucleic acid is not particularly limited and may be appropriately selected depending on the intended purpose. Examples of the nucleic acid include DNA, RNA, and cDNA.

The nucleic acid or nucleic acid fragment may be a natural product obtained from an organism, or a processed product of the natural product, or a product produced by utilizing a genetic recombination technique, or a chemically synthesized artificially synthesized nucleic acid molecule. One of these nucleic acids may be used alone or two or more of these nucleic acids may be used in combination. With the artificially synthesized nucleic acid molecule, it is possible to suppress impurities and set the molecular weight to a low level. This makes it possible to improve the initial reaction efficiency.

The artificially synthesized nucleic acid means an artificially synthesized nucleic acid produced to have the same composition (base, deoxyribose, and phosphoric acid) as naturally existent DNA or RNA. Examples of the artificially synthesized nucleic acid include not only a nucleic acid having a nucleotide sequence coding a protein, but also a nucleic acid having an arbitrary nucleotide sequence.

The form of the nucleic acid is not particularly limited and may be appropriately selected depending on the intended purpose. Examples of the form of the nucleic acid include double-strand nucleic acid, single-strand nucleic acid, and partially double-strand or single-strand nucleic acid. Circular or linear plasmids can also be used. The nucleic acid may be modified or mutated.

It is applicable that the nucleic acid have a specific nucleotide sequence. The term “specific” means “particularly specified”.

The specific nucleotide sequence is not particularly limited and may be appropriately selected depending on the intended purpose. Examples of the specific nucleotide sequence include nucleotide sequences used for infectious disease testing, naturally non-existent non-natural nucleotide sequences, animal cell-derived nucleotide sequences, plant cell-derived nucleotide sequences, fungal cell-derived nucleotide sequences, bacterium-derived nucleotide sequences, and virus-derived nucleotide sequences. One of these nucleotide sequences may be used alone or two or more of these nucleotide sequences may be used in combination.

When using the non-natural nucleotide sequence, the specific nucleotide sequence preferably has a GC content of 30% or higher but 70% or lower, and preferably has a constant GC content (for example, see SEQ ID NO. 1).

The nucleotide length of the specific nucleotide sequence is not particularly limited, may be appropriately selected depending on the intended purpose, and may be, for example, a nucleotide length of 20 base pairs (or mer) or greater but 10,000 base pairs (or mer) or less.

When using the nucleotide sequence used for infectious disease testing, the nucleotide sequence is not particularly limited and may be appropriately selected depending on the intended purpose as long as the nucleotide sequence includes a nucleotide sequence specific to the intended infectious disease. It is applicable that the nucleotide sequence include a nucleotide sequence designated in official analytical methods or officially announced methods (for example, see SEQ ID NOS. 2 and 3).

The nucleic acid may be a nucleic acid derived from the cells to be used, or a nucleic acid introduced by transgenesis. When a nucleic acid introduced by transgenesis and a plasmid are used as the nucleic acid, it is applicable to confirm that one copy of the nucleic acid is introduced per cell. The method for confirming that one copy of the nucleic acid is introduced is not particularly limited and may be appropriately selected depending on the intended purpose. Examples of the method include DNA sequencing, a PCR method, and a Southern blotting method.

One kind or two or more kinds of nucleic acids having specific nucleotide sequences may be introduced by transgenesis. Also in the case of introducing only one kind of a nucleic acid by transgenesis, nucleotide sequences of the same kind may be introduced in tandem depending on the intended purpose.

The method for transgenesis is not particularly limited and may be appropriately selected depending on the intended purpose as long as the method can introduce an intended copy number of specific nucleic acid sequences at an intended position. Examples of the method include homologous recombination, CRISPR/Cas9, CRISPR/Cpf1, TALEN, Zinc finger nuclease, Flip-in, and Jump-in. In the case of yeast fungi, homologous recombination is preferable among these methods in terms of a high efficiency and ease of controlling.

—Carrier—

It is applicable to handle the amplifiable reagent in a state of being carried on a carrier. When the amplifiable reagent is a nucleic acid, a preferable form is the nucleic acid being carried (or preferably encapsulated) by the carrier having a particle shape (carrier particles).

The carrier is not particularly limited and may be appropriately selected depending on the intended purpose. Examples of the carrier include cells, resins, phages, viruses, liposomes, and microcapsules.

—Cells—

The cell means a structural, functional unit that includes an amplifiable reagent (for example, a nucleic acid) and forms an organism.

The cells are not particularly limited and may be appropriately selected depending on the intended purpose. All kinds of cells can be used regardless of whether the cells are eukaryotic cells, prokaryotic cells, multicellular organism cells, and unicellular organism cells. One of these kinds of cells may be used alone or two or more of these kinds of cells may be used in combination.

The eukaryotic cells are not particularly limited and may be appropriately selected depending on the intended purpose. Examples of the eukaryotic cells include animal cells, insect cells, plant cells, fungi, algae, and protozoans. One of these kinds of eukaryotic cells may be used alone or two or more of these kinds of eukaryotic cells may be used in combination. Among these eukaryotic cells, animal cells and fungi are preferable.

The adherent cells may be primary cells directly taken from tissues or organs, or may be cells obtained by passaging primary cells directly taken from tissues or organs a few times. Adherent cells may be appropriately selected depending on the intended purpose. Examples of adherent cells include differentiated cells and undifferentiated cells.

The differentiated cells are not particularly limited and may be appropriately selected depending on the intended purpose. Examples of differentiated cells include: hepatocytes, which are parenchymal cells of a liver; stellate cells; Kupffer cells; endothelial cells such as vascular endothelial cells, sinusoidal endothelial cells, and corneal endothelial cells; fibroblasts; osteoblasts; osteoclasts; periodontal ligamentderived cells; epidermal cells such as epidermal keratinocytes; epithelial cells such as tracheal epithelial cells, intestinal epithelial cells, cervical epithelial cells, and corneal epithelial cells; mammary glandular cells; pericytes; muscle cells such as smooth muscle cells and myocardial cells; renal cells; pancreatic islet cells; nerve cells such as peripheral nerve cells and optic nerve cells; chondrocytes; and bone cells.

The undifferentiated cells are not particularly limited and may be appropriately selected depending on the intended purpose. Examples of undifferentiated cells include: pluripotent stem cells such as embryotic stem cells, which are undifferentiated cells, and mesenchymal stem cells having pluripotency; unipotent stem cells such as vascular endothelial progenitor cells having unipotency; and iPS cells.

The fungi are not particularly limited and may be appropriately selected depending on the intended purpose. Examples of fungi include molds and yeast fungi. One of these kinds of fungi may be used alone or two or more of these kinds of fungi may be used in combination. Among these kinds of fungi, yeast fungi are preferable because the cell cycles are adjustable and monoploids can be used.

The cell cycle means a cell proliferation process in which cells undergo cell division and cells (daughter cells) generated by the cell division become cells (mother cells) that undergo another cell division to generate new daughter cells.

The yeast fungi are not particularly limited and may be appropriately selected depending on the intended purpose. For example, Bar1-deficient yeasts with enhanced sensitivity to a pheromone (sex hormone) that controls the cell cycle at a G1 phase are preferable. When yeast fungi are Bar1-deficient yeasts, the abundance ratio of yeast fungi with uncontrolled cell cycles can be reduced. This makes it possible to, for example, prevent the amplifiable reagent from increasing in number in the cells contained in a well.

The prokaryotic cells are not particularly limited and may be appropriately selected depending on the intended purpose. Examples of the prokaryotic cells include eubacteria and archaea. One of these kinds of prokaryotic cells may be used alone or two or more of these kinds of prokaryotic cells may be used in combination.

As the cells, dead cells are preferable. With the dead cells, it is possible to prevent occurrence of cell division after fractionation.

As the cells, cells that can emit light upon reception of light are preferable. With cells that can emit light upon reception of light, it is possible to land the cells into wells while having a highly accurate control on the number of cells.

The reception of light means receiving of light.

The optical sensor means a passive sensor configured to collect, with a lens, any light in the range from visible light rays visible by human eyes to near infrared rays, shortwavelength infrared rays, and thermal infrared rays that have longer wavelengths than the visible light rays, to obtain, for example, shapes of target cells in the form of image data.

—Cells that can Emit Light Upon Reception of Light—

The cells that can emit light upon reception of light are not particularly limited and may be appropriately selected depending on the intended purpose as long as the cells can emit light upon reception of light. Examples of the cells include cells stained with a fluorescent dye, cells expressing a fluorescent protein, and cells labeled with a fluorescent-labeled antibody.

The cellular site stained with a fluorescent dye, expressing a fluorescent protein, or labeled with a fluorescent-labeled antibody is not particularly limited. Examples of the cellular site include a whole cell, a cell nucleus, and a cellular membrane.

—Fluorescent Dye—

Examples of the fluorescent dye include fluoresceins, azo dyes, rhodamines, coumarins, pyrenes, cyanines. One of these fluorescent dyes may be used alone or two or more of these fluorescent dyes may be used in combination. Among these fluorescent dyes, fluoresceins, azo dyes, and rhodamines are preferable, and eosin, Evans blue, trypan blue, rhodamine 6G, rhodamine B, and rhodamine 123 are more preferable.

As the fluorescent dye, a commercially available product may be used. Examples of the commercially available product include product name: EOSIN Y (Wako Pure Chemical Industries, Ltd.), product name: EVANS BLUE (Wako Pure Chemical Industries, Ltd.), product name: TRYPAN BLUE (Wako Pure Chemical Industries, Ltd.), product name: RHODAMINE 6G (Wako Pure Chemical Industries, Ltd.), product name: RHODAMINE B (Wako Pure Chemical Industries, Ltd.), and product name: RHODAMINE 123 (Wako Pure Chemical Industries, Ltd.).

—Fluorescent Protein—

Examples of the fluorescent protein include Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, mPlum, PS-CFP, Dendra2, Kaede, EosFP, and KikumeGR. One of these fluorescent proteins may be used alone or two or more of these fluorescent proteins may be used in combination.

—Fluorescent-Labeled Antibody—

The fluorescent-labeled antibody is not particularly limited and may be appropriately selected depending on the intended purpose as long as the fluorescent-labeled antibody is fluorescent-labeled. Examples of the fluorescent-labeled antibody include CD4-FITC and CD8-PE. One of these fluorescent-labeled antibodies may be used alone or two or more of these fluorescent-labeled antibodies may be used in combination.

The volume average particle diameter of the cells is in the following order of preference (from lowest to highest): 30 micrometers or less, 10 micrometers or less, and 7 micrometers or less in a free state. When the volume average particle diameter of the cells is 30 micrometers or less, the cells can be suitably used in an inkjet method or a liquid droplet discharging unit such as a cell sorter.

The volume average particle diameter of the cells can be measured by, for example, a measuring method described below.

Ten microliters is extracted from a produced stained yeast dispersion liquid and poured onto a plastic slide formed of polymethyl methacrylate (PMMA). Then, with an automated cell counter (product name: COUNTESS AUTOMATED CELL COUNTER, Invitrogen), the volume average particle diameter of the cells can be measured. The cell number can be obtained by a similar measuring method.

The concentration of the cells in the cell suspension is not particularly limited, may be appropriately selected depending on the intended purpose, and is in the following order of preference (from lowest to highest): 5×10⁴ cells/mL or higher but 5×10⁸ cells/mL or lower, and 5×10⁴ cells/mL or higher but 5×10⁷ cells/mL or lower. When the cell number is 5×10⁴ cells/mL or higher but 5×10⁸ cells/mL or lower, it can be ensured that cells be contained in a discharged liquid droplet without fail. The cell number can be measured with an automated cell counter (product name: COUNTESS AUTOMATED CELL COUNTER, Invitrogen) in the same manner as measuring the volume average particle diameter.

The cell number of the cells including a nucleic acid is not particularly limited and may be appropriately selected depending on the intended purpose as long as the cell number is a plural number.

—Resin—

The material, the shape, the size, and the structure of the resin are not particularly limited and may be appropriately selected depending on the intended purpose as long as the resin can carry the amplifiable reagent (for example, a nucleic acid).

—Liposome—

The liposome is a lipid vesicle formed of a lipid bilayer containing lipid molecules. Specifically, the liposome means a lipid-containing closed vesicle including a space separated from the external environment by a lipid bilayer produced based on the polarities of a hydrophobic group and a hydrophilic group of lipid molecules.

The liposome is a closed vesicle formed of a lipid bilayer using a lipid, and contains an aqueous phase (internal aqueous phase) in the space in the closed vesicle. The internal aqueous phase contains, for example, water. The liposome may be singlelamellar (single-layer lamellar or unilamellar with a single bilayer) or multilayer lamellar (multilamellar, with an onion-like structure including multiple bilayers, with the individual layers separated by watery layers).

As the liposome, a liposome that can encapsulate an amplifiable reagent (for example, a nucleic acid) is preferable. The form of encapsulation is not particularly limited. “Encapsulation” means a form of a nucleic acid being contained in the internal aqueous phase and the layer of the liposome. Examples of the form include a form of encapsulating a nucleic acid in the closed space formed of the layer, a form of encapsulating a nucleic acid in the layer per se, and a combination of these forms.

The size (average particle diameter) of the liposome is not particularly limited as long as the liposome can encapsulate an amplifiable reagent (for example, a nucleic acid). It is applicable that the liposome have a spherical form or a form close to the spherical form.

The component (layer component) constituting the lipid bilayer of the liposome is selected from lipids. As the lipid, an arbitrary lipid that can dissolve in a mixture solvent of a water-soluble organic solvent and an ester-based organic solvent can be used. Specific examples of the lipid include phospholipids, lipids other than phospholipids, cholesterols, and derivatives of these lipids. These components may be formed of a single kind of a component or a plurality of kinds of components.

—Microcapsule—

The microcapsule means a minute particle having a wall material and a hollow structure, and can encapsulate an amplifiable reagent (for example, a nucleic acid) in the hollow structure.

The microcapsule is not particularly limited, and, for example, the wall material and the size of the microcapsule may be appropriately selected depending on the intended purpose.

Examples of the wall material of the microcapsule include polyurethane resins, polyurea, polyurea-polyurethane resins, urea-formaldehyde resins, melamineformaldehyde resins, polyamide, polyester, polysulfone amide, polycarbonate, polysulfinate, epoxyr, acrylic acid ester, methacrylic acid ester, vinyl acetate, and gelatin. One of these wall materials may be used alone or two or more of these wall materials may be used in combination.

The size of the microcapsule is not particularly limited and may be appropriately selected depending on the intended purpose as long as the microcapsule can encapsulate an amplifiable reagent (for example, a nucleic acid).

The method for producing the microcapsule is not particularly limited and may be appropriately selected depending on the intended purpose. Examples of the method include an in-situ method, an interfacial polymerization method, and a coacervation method.

The device of the present disclosure includes at least one well, and preferably includes an identifier unit, and further includes other components as needed.

In the present disclosure, a plate may include not only wells to be filled with the nucleic acid having at least one of a full-length nucleotide sequence and a partial nucleotide sequence of rRNA or rDNA in a defined copy number, but also wells to be filled with the testing target sample (the wells to be filled with the testing target sample may also be filled with an amplifiable reagent as described above). Wells in general will be described below.

<Well>

For example, the shape, the number, the volume, the material, and the color of the well are not particularly limited and may be appropriately selected depending on the intended purpose.

The shape of the well is not particularly limited and may be appropriately selected depending on the intended purpose as long as, for example, a nucleic acid can be located in the well. Examples of the shape of the well include: concaves such as a flat bottom, a round bottom, a U bottom, and a V bottom; and sections on a substrate.

The number of the wells is in the following order of preference (from lowest to highest): at least 1, a plural number of 2 or greater, 5 or greater, and 50 or greater.

Examples with the number of the wells of 1 include a PCR tube.

As an example, with the number of the wells of 2 or greater, a multi-well plate is suitably used.

Examples of the multi-well plate include a 24-well, 48-well, 96-well, 384-well, or 1,536-well plate.

The volume of the well is not particularly limited, may be appropriately selected depending on the intended purpose, and is preferably 10 microliters or greater but 1,000 microliters or less in consideration of the amount of a sample used in a common nucleic acid testing device.

The material of the well is not particularly limited and may be appropriately selected depending on the intended purpose. Examples of the material of the well include polystyrene, polypropylene, polyethylene, fluororesins, acrylic resins, polycarbonate, polyurethane, polyvinyl chloride, and polyethylene terephthalate.

Examples of the color of the well include transparent colors, semi-transparent colors, chromatic colors, and complete light-shielding colors.

Wettability of the well is not particularly limited and may be appropriately selected depending on the intended purpose. The wettability of the well is preferably water repellency. When the wettability of the well is water repellency, adsorption of the nucleic acid having at least one of a full-length nucleotide sequence and a partial nucleotide sequence of rRNA or rDNA to the internal wall of the well can be reduced. Further, when the wettability of the well is water repellency, the nucleic acid having at least one of a full-length nucleotide sequence and a partial nucleotide sequence of rRNA or rDNA, a set of primers, and an amplification reagent in the well can be added in a state of a solution.

The method for imparting water repellency to the internal wall of the well is not particularly limited and may be appropriately selected depending on the intended purpose. Examples of the method include a method of forming a fluororesin coating film, a fluorine plasma treatment, and an embossing treatment. Particularly, by applying a water repellency imparting treatment that imparts a contact angle of 100 degrees or greater, it is possible to suppress reduction of the nucleic acid having at least one of a full-length nucleotide sequence and a partial nucleotide sequence of rRNA or rDNA due to spill of the liquid and suppress increase of uncertainty (or coefficient of variation).

<Base Material>

The device is preferably a plate-shaped device obtained by providing a well in a base material but may be linking-type well tubes such as 8-series tubes.

For example, the material, the shape, the size, and the structure of the base material are not particularly limited and may be appropriately selected depending on the intended purpose.

The material of the base material is not particularly limited and may be appropriately selected depending on the intended purpose. Examples of the material of the base material include semiconductors, ceramics, metals, glass, quartz glass, and plastics.

Among these materials, plastics are preferable.

Examples of the plastics include polystyrene, polypropylene, polyethylene, fluororesins, acrylic resins, polycarbonate, polyurethane, polyvinyl chloride, and polyethylene terephthalate.

The shape of the base material is not particularly limited and may be appropriately selected depending on the intended purpose. For example, board shapes and plate shapes are preferable.

The structure of the base material is not particularly limited, may be appropriately selected depending on the intended purpose, and may be, for example, a single-layer structure or a multilayered structure.

<Identifier Unit>

It is applicable that the device include an identifier unit that enables identifying information on a coefficient of variation CV of the nucleic acid having at least one of a full-length nucleotide sequence and a partial nucleotide sequence of rRNA or rDNA in the defined copy number in the well and information on uncertainty. The information on a CV value and the information on uncertainly will be described in detail below.

The identifier unit is not particularly limited and may be appropriately selected depending on the intended purpose. Examples of the identifier unit include a memory, an IC chip, a barcode, a QR code (registered trademark), a Radio Frequency Identifier (hereinafter may also be referred to as “RFID”), color coding, and printing.

The position at which the identifier unit is provided and the number of identifier units are not particularly limited and may be appropriately selected depending on the intended purpose.

Examples of the information to be stored in the identifier unit include not only an existence probability at which the nucleic acid having at least one of a full-length nucleotide sequence and a partial nucleotide sequence of rRNA or rDNA in the defined copy number exists in a well in the defined copy number, but also results of analyses (for example, activity value and emission intensity), the number of nucleic acids having at least one of a full-length nucleotide sequence and a partial nucleotide sequence of rRNA or rDNA (for example, the number of cells), whether cells are alive or dead, a copy number of a specific nucleotide sequence, which of a plurality of wells is filled with the nucleic acid having at least one of a full-length nucleotide sequence and a partial nucleotide sequence of rRNA or rDNA in the defined copy number, the kind of the nucleic acid having at least one of a full-length nucleotide sequence and a partial nucleotide sequence of rRNA or rDNA in the defined copy number, the measurement date and time, and the name of the person in charge of measurement.

The information stored in the identifier unit can be read with various kinds of reading units. For example, when the identifier unit is a barcode, a barcode reader is used as the reading unit.

The method for writing information in the identifier unit is not particularly limited and may be appropriately selected depending on the intended purpose. Examples of the method include manual input, a method of directly writing data through a liquid droplet forming device configured to count the number of nucleic acids having at least one of a full-length nucleotide sequence and a partial nucleotide sequence of rRNA or rDNA in the defined copy number during dispensing of nucleic acids having at least one of a full-length nucleotide sequence and a partial nucleotide sequence of rRNA or rDNA in the defined copy number into the wells, transfer of data stored in a server, and transfer of data stored in a cloud system.

<Other Components>

The other components are not particularly limited and may be appropriately selected depending on the intended purpose. Examples of the other components include a sealing member.

—Sealing Member—

It is applicable that the device include a sealing member in order to prevent mixing of foreign matters into the wells and outflow of the filled materials.

It is applicable that the sealing member be configured to be capable of sealing an opening of at least one well and separable at a perforation in order to be capable of sealing or opening each one of the wells individually.

The shape of the sealing member is preferably a cap shape matching the inner diameter of a well, or a film shape for covering the well opening.

Examples of the material of the sealing member include polyolefin resins, polyester resins, polystyrene resins, and polyamide resins.

It is applicable that the sealing member have a film shape that can seal all wells at a time. It is also applicable that the sealing member be configured to have different adhesive strengths for wells that need to be reopened and wells that need not, in order that the user can reduce improper use.

It is applicable that the well contain at least one primer and an amplification reagent.

The primer is a synthetic oligonucleotide having a complementary nucleotide sequence that includes 18 or more but 30 or less nucleotides and is specific to a template DNA of a polymerase chain reaction (PCR). A pair of primers, namely a forward primer and a reverse primer, are set at two positions in a manner to sandwich the region to be amplified.

Examples of the amplification reagent for, for example, a polymerase chain reaction (PCR) include enzymes such as DNA polymerase, matrices such as the four kinds of bases (dGTP, dCTP, dATP, and dTTP), Mg²⁺ (2 mM magnesium chloride), and a buffer for maintaining the optimum pH (pH of from 7.5 through 9.5).

The state of the nucleic acid having at least one of a full-length nucleotide sequence and a partial nucleotide sequence of rRNA or rDNA, a primer, and an amplification reagent in the well is not particularly limited and may be appropriately selected depending on the intended purpose. For example, the state of the nucleic acid having at least one of a full-length nucleotide sequence and a partial nucleotide sequence of rRNA or rDNA, a primer, and an amplification reagent may be a state of either a solution or a solid. In terms of convenience of use, the state of the nucleic acid having at least one of a full-length nucleotide sequence and a partial nucleotide sequence of rRNA or rDNA, a primer, and an amplification reagent is particularly preferably a state of a solution. In a state of a solution, a user can use the nucleic acid having at least one of a full-length nucleotide sequence and a partial nucleotide sequence of rRNA or rDNA, a primer, and an amplification reagent for a test immediately. In terms of transportation, the state of the nucleic acid having at least one of a full-length nucleotide sequence and a partial nucleotide sequence of rRNA or rDNA, a primer, and an amplification reagent is particularly preferably a state of a solid and more preferably a solid dry state. In a solid dry state, a reaction speed at which the nucleic acid having at least one of a full-length nucleotide sequence and a partial nucleotide sequence of rRNA or rDNA is decomposed by, for example, a breakdown enzyme, can be reduced, and storage stability of the nucleic acid having at least one of a full-length nucleotide sequence and a partial nucleotide sequence of rRNA or rDNA, a primer, and an amplification reagent can be improved.

It is applicable that the nucleic acid having at least one of a full-length nucleotide sequence and a partial nucleotide sequence of rRNA or rDNA, a primer, and an amplification reagent be filled in appropriate amounts in the device in the solid dry state, in order to make it possible to use the nucleic acid having at least one of a full-length nucleotide sequence and a partial nucleotide sequence of rRNA or rDNA, a primer, and an amplification reagent in the form of a reaction solution immediately by dissolving the nucleic acid having at least one of a full-length nucleotide sequence and a partial nucleotide sequence of rRNA or rDNA, a primer, and an amplification reagent in a buffer or water immediately before use of the device.

The method for drying the nucleic acid having at least one of a full-length nucleotide sequence and a partial nucleotide sequence of rRNA or rDNA, a primer, and an amplification reagent is not particularly limited and may be appropriately selected depending on the intended purpose. Examples of the drying method include freeze drying, heating drying, hot-air drying, vacuum drying, steam drying, suction drying, infrared drying, barrel drying, and spin drying.

A coefficient of variation of the well expressed by CV value is in the following order of preference (from lowest to highest): 20% or lower, and 10% or lower.

It is applicable that the well have information on the specific number and uncertainty based on the specific number.

The CV value and the information on uncertainty will be described below.

Solute molecules of, for example, a nucleic acid sample, while being dissolved in solvent molecules, migrate through the solvent molecules due to thermal fluctuation. In this case, the distribution state of the molecules is generally said to conform to a Poisson distribution. This indicates that the number of molecules in the solution filled in a container has a distribution, i.e., a variation (coefficient of variation), regardless of with what level of accuracy the solution having a prescribed concentration is weighed out and filled in the container.

Here, the coefficient of variation means a relative value of the variation in the number of nucleic acids filled in each concave, where the variation occurs when nucleic acids are filled in the concave. That is, the coefficient of variation means the coefficient of variation for the number of nucleic acids filled in the concave. The coefficient of variation is a value obtained by dividing standard deviation σ by an average value x. Here, the coefficient of variation CV is assumed to be a value obtained by dividing standard deviation σ by an average copy number (average number of copies filled) x. In this case, a relational expression represented by Formula 1 below is established.

$$\begin{matrix}[ Math . 1 ] & \\ CV = \frac{σ}{x} & Formula 1\end{matrix}$$

Generally, nucleic acids have a random distribution state of a Poisson distribution in a dispersion liquid. Therefore, in a random distribution state by a serial dilution method, i.e., of a Poisson distribution, standard deviation σ can be regarded as satifying a relational expression represented by Formula 2 below with an average copy number x. Hence, in the case where a dispersion liquid of nucleic acids is diluted by a serial dilution method, when coefficients of variation CV (CV values) for average copy numbers x are calculated according to Formula 3 below derived from Formula 1 above and Formula 2 based on the standard deviation σ and the average copy numbers x, the results are as presented in Table 1 and FIG. 7.

[Math.2]

σ=\sqrt{x} Formula 2

$$\begin{matrix}[ Math . 3 ] & \\ CV = \frac{1}{\sqrt{x}} & Formula 3\end{matrix}$$

TABLE 1

| Average copy number x | Coefficient of variation CV |
|---|---|
| 1.00E+00 | 100.00% |
| 1.00E+01 | 31.62% |
| 1.00E+02 | 10.00% |
| 1.00E+03 | 3.16% |
| 1.00E+04 | 1.00% |
| 1.00E+05 | 0.32% |
| 1.00E+06 | 0.10% |
| 1.00E+07 | 0.03% |
| 1.00E+08 | 0.01% |

From the results of Table 1 and FIG. 7, it can be understood that when a well is to be filled with, for example, the nucleic acid having at least one of a full-length nucleotide sequence and a partial nucleotide sequence of rRNA or rDNA in a copy number of 100 by a serial dilution method, the final copy number of the nucleic acid having at least one of a full-length nucleotide sequence and a partial nucleotide sequence of rRNA or rDNA to be filled in the reaction solution has a coefficient of variation (CV) of at least 10%, even when other accuracies are ignored.

The coefficient of variation is a value obtained by dividing standard deviation σ by an average copy number x. “CV value” is used as abbreviation. The coefficient of variation CV for a copy number having variation according to a Poisson distribution can be obtained from FIG. 7.

As regards the number of the nucleic acid having at least one of a full-length nucleotide sequence and a partial nucleotide sequence of rRNA or rDNA in the well, it is applicable that the well have information on uncertainty based on the defined copy number.

Uncertainty is defined in ISO/IEC Guide 99:2007 [International Vocabulary of Metrology-Basics and general concepts and related terms (VIM)] as “a parameter that characterizes measurement result-incidental variation or dispersion of values rationally linkable to the measured quantity”. Here, “values rationally linkable to the measured quantity” means candidates for the true value of the measured quantity. That is, uncertainty means information on the variation of the results of measurement due to operations and devices involved in production of a measurement target. With a greater uncertainty, a greater variation is predicted in the results of measurement.

For example, the uncertainty may be standard deviation obtained from the results of measurement, or a half value of a reliability level, which is expressed as a numerical range in which the true value is contained at a predetermined probability or higher. The uncertainty may be calculated according to the methods based on, for example, Guide to the Expression of Uncertainty in Measurement (GUM:ISO/IEC Guide 98-3), and Japan Accreditation Board Note 10, Guideline on Uncertainty in Measurement in Test. As the method for calculating the uncertainty, for example, there are two types of applicable methods: a type-A evaluation method using, for example, statistics of the measured values, and a type-B evaluation method using information on uncertainty obtained from, for example, calibration certificate, manufacturer's specification, and information open to the public.

All uncertainties due to factors such as operations and measurement can be expressed by the same reliability level, by conversion of the uncertainties to standard uncertainty. Standard uncertainty indicates variation in the average value of measured values. In an example method for calculating the uncertainty, for example, factors that may cause uncertainties are extracted, and uncertainties (standard deviations) due to the respective factors are calculated. Then, the calculated uncertainties due to the respective factors are synthesized according to the sum-of-squares method, to calculate a synthesized standard uncertainty. In the calculation of the synthesized standard uncertainty, the sum-of-squares method is used. Therefore, a factor that causes a sufficiently small uncertainty can be ignored, among the factors that cause uncertainties. There are some conceivable factors that cause uncertainty. For example, in a production process of introducing the intended nucleic acid into cells and dispensing the cells while counting the number of cells, examples of the factors of uncertainties of the number of the intended nucleic acid having at least one of a full-length nucleotide sequence and a partial nucleotide sequence of rRNA or rDNA in each well include the number of nucleic acids having at least one of a full-length nucleotide sequence and a partial nucleotide sequence of rRNA or rDNA in a cell (e.g., the cell cycle of the cell), the unit configured to locate cells over a plate (including any outcomes of operations of an inkjet device or each section of the device, such as operation timings of the device, e.g., the number of cells included in a liquid droplet when a cell suspension is formed into a liquid droplet shape), the frequency at which located cells are located at appropriate positions of the plate (e.g., the number of cells located in a well), and contamination of the reagent.

When obtaining the coefficient of variation CV by dividing uncertainty (standard variation σ) by average defined copy number x, the calculation may be based on experimental results of average defined copy numbers of the nucleic acid having at least one of a full-length nucleotide sequence and a partial nucleotide sequence of rRNA or rDNA in the defined copy number and uncertainties.

<Method for Producing Device>

A method for producing a device containing the nucleic acid having at least one of a full-length nucleotide sequence and a partial nucleotide sequence of rRNA or rDNA in a defined copy number will be described below.

The method for producing a device of the present disclosure includes a cell suspension producing step of producing a cell suspension containing a plurality of cells including a nucleic acid having at least one of a full-length nucleotide sequence and a partial nucleotide sequence of rRNA or rDNA, and a solvent, a liquid droplet landing step of discharging the cell suspension in the form of liquid droplets to sequentially land the liquid droplets in wells of a plate, a cell number counting step of counting the number of cells contained in the liquid droplets with a sensor after the liquid droplets are discharged and before the liquid droplets land in the wells, and a nucleic acid extracting step of extracting nucleic acids from cells in the wells, preferably includes a step of calculating the degrees of certainty of estimated numbers of nucleic acids in the cell suspension producing step, the liquid droplet landing step, and the cell number counting step, an outputting step, and a recording step, and further includes other steps as needed.

<<Cell Suspension Producing Step>>

The cell suspension producing step is a step of producing a cell suspension containing a plurality of cells including a nucleic acid having at least one of a full-length nucleotide sequence and a partial nucleotide sequence of rRNA or rDNA, and a solvent.

The solvent means a liquid used for dispersing cells.

Suspension in the cell suspension means a state of cells being present dispersedly in the solvent.

Producing means a producing operation.

—Cell Suspension—

The cell suspension contains a plurality of cells including a nucleic acid having at least one of a full-length nucleotide sequence and a partial nucleotide sequence of rRNA or rDNA and a solvent, preferably contains an additive, and further contains other components as needed.

The plurality of cells including a nucleic acid having at least one of a full-length nucleotide sequence and a partial nucleotide sequence of rRNA or rDNA are as described above.

—Solvent—

The solvent is not particularly limited and may be appropriately selected depending on the intended purpose. Examples of the solvent include water, a culture fluid, a separation liquid, a diluent, a buffer, an organic matter dissolving liquid, an organic solvent, a polymeric gel solution, a colloid dispersion liquid, an electrolytic aqueous solution, an inorganic salt aqueous solution, a metal aqueous solution, and mixture liquids of these liquids. One of these solvents may be used alone or two or more of these solvents may be used in combination. Among these solvents, water and a buffer are preferable, and water, a phosphate buffered saline (PBS), and a Tris-EDTA buffer (TE) are preferable.

—Additive—

An additive is not particularly limited and may be appropriately selected depending on the intended purpose. Examples of the additive include a surfactant, a nucleic acid, and a resin. One of these additives may be used alone or two or more of these additives may be used in combination.

The surfactant can prevent mutual aggregation of cells and improve continuous discharging stability.

The surfactant is not particularly limited and may be appropriately selected depending on the intended purpose. Examples of the surfactant include ionic surfactants and nonionic surfactants. One of these surfactants may be used alone or two or more of these surfactants may be used in combination. Among these surfactants, nonionic surfactants are preferable because proteins are neither modified nor deactivated by nonionic surfactants, although depending on the addition amount of the nonionic surfactants.

Examples of the ionic surfactants include fatty acid sodium, fatty acid potassium, alpha-sulfo fatty acid ester sodium, sodium straight-chain alkyl benzene sulfonate, alkyl sulfuric acid ester sodium, alkyl ether sulfuric acid ester sodium, and sodium alpha-olefin sulfonate. One of these ionic surfactants may be used alone or two or more of these ionic surfactants may be used in combination. Among these ionic surfactants, fatty acid sodium is preferable and sodium dodecyl sulfonate (SDS) is preferable.

Examples of the nonionic surfactants include alkyl glycoside, alkyl polyoxyethylene ether (e.g., BRIJ series), octyl phenol ethoxylate (e.g., TRITON X series, IGEPAL CA series, NONIDET P series, and NIKKOL OP series), polysorbates (e.g., TWEEN series such as TWEEN 20), sorbitan fatty acid esters, polyoxyethylene fatty acid esters, alkyl maltoside, sucrose fatty acid esters, glycoside fatty acid esters, glycerin fatty acid esters, propylene glycol fatty acid esters, and fatty acid monoglyceride. One of these nonionic surfactants may be used alone or two or more of these nonionic surfactants may be used in combination. Among these nonionic surfactants, polysorbates are preferable.

The content of the surfactant is not particularly limited, may be appropriately selected depending on the intended purpose, and is preferably 0.001% by mass or greater but 30% by mass or less relative to the total amount of the cell suspension. When the content of the surfactant is 0.001% by mass or greater, an effect of adding the surfactant can be obtained. When the content of the surfactant is 30% by mass or less, aggregation of cells can be suppressed, making it possible to accurately control the number of nucleic acid molecules in the cell suspension.

The nucleic acid is not particularly limited and may be appropriately selected depending on the intended purpose as long as the nucleic acid does not affect the detection target nucleic acid. Examples of the nucleic acid include ColE1 DNA. With such a nucleic acid, it is possible to prevent the nucleic acid having at least one of a full-length nucleotide sequence and a partial nucleotide sequence of rRNA or rDNA from adhering to the wall surface of a well.

The resin is not particularly limited and may be appropriately selected depending on the intended purpose. Examples of the resin include polyethyleneimide.

—Other Materials—

Other materials are not particularly limited and may be appropriately selected depending on the intended purpose. Examples of the other materials include a crosslinking agent, a pH adjustor, an antiseptic, an antioxidant, an osmotic pressure regulator, a humectant, and a dispersant.

<Method for Dispersing Cells>

The method for dispersing the cells is not particularly limited and may be appropriately selected depending on the intended purpose. Examples of the method include a medium method such as a bead mill, an ultrasonic method such as an ultrasonic homogenizer, and a method using a pressure difference such as a French press. One of these methods may be used alone or two or more of these methods may be used in combination. Among these methods, the ultrasonic method is preferable because the ultrasonic method has low damage on the cells. With the medium method, a high crushing force may destroy cellular membranes or cell walls, and the medium may mix as contamination.

<Method for Screening Cells>

The method for screening the cells is not particularly limited and may be appropriately selected depending on the intended purpose. Examples of the method include screening by wet classification, a cell sorter, and a filter. One of these methods may be used alone or two or more of these methods may be used in combination. Among these methods, screening by a cell sorter and a filter is preferable because the method has low damage on the cells.

It is applicable to estimate the number of nucleic acids having at least one of a full-length nucleotide sequence and a partial nucleotide sequence of rRNA or rDNA from the cell number contained in the cell suspension, by measuring the cell cycles of the cells.

Measuring the cell cycles means quantifying the cell number due to cell division. Estimating the number of nucleic acids means obtaining the copy number of nucleic acids (the number of nucleic acid molecules) based on the cell number.

What is to be counted need not be the cell number, but may be the number of nucleic acids having at least one of a full-length nucleotide sequence and a partial nucleotide sequence of rRNA or rDNA. Typically, it is safe to consider that the number of nucleic acids having at least one of a full-length nucleotide sequence and a partial nucleotide sequence of rRNA or rDNA is equal to the cell number, because a nucleic acid region that is not fully included per cell is selected as the nucleic acid having at least one of a full-length nucleotide sequence and a partial nucleotide sequence of rRNA or rDNA and introduced per cell by gene recombination. However, nucleic acid replication occurs in cells in order for the cells to undergo cell division at specific cycles. Cell cycles are different depending on the kinds of cells. By extracting a predetermined amount of the solution from the cell suspension and measuring the cycles of a plurality of cells, it is possible to calculate an expected value of the number of nucleic acids having at least one of a full-length nucleotide sequence and a partial nucleotide sequence of rRNA or rDNA included in one cell and the degree of certainty of the estimated value. This can be realized by, for example, observing nuclear stained cells with a flow cytometer.

Degree of certainty means a probability of occurrence of one specific event, predicted beforehand, when there are possibilities of occurrence of some events.

Calculation means deriving a needed value by a calculating operation.

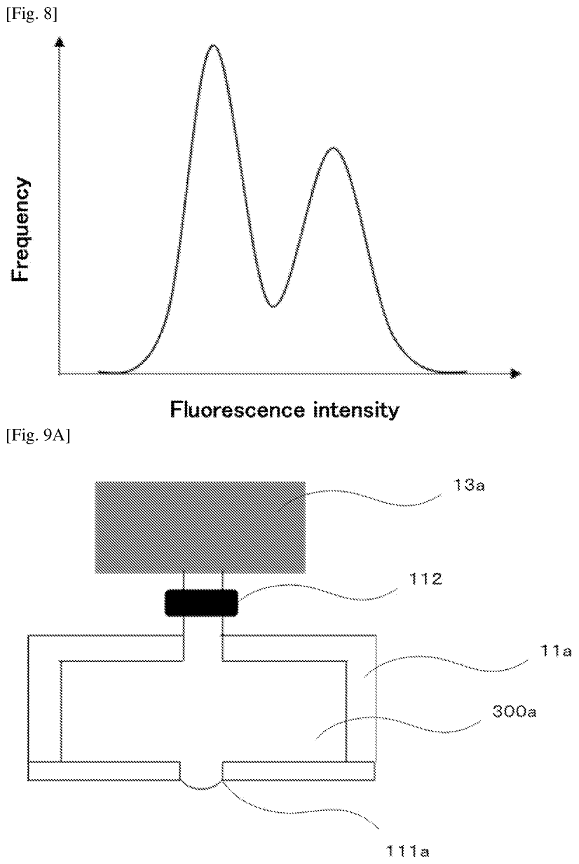

FIG. 8 is a graph plotting an example of a relationship between the frequency and the fluorescence intensity of cells in which DNA replication has occurred. As plotted in FIG. 8, based on presence or absence of replication of the nucleic acid having at least one of a full-length nucleotide sequence and a partial nucleotide sequence of rRNA or rDNA, two peaks appear on the histogram. Hence, the percentage of presence of cells in which DNA replication has occurred can be calculated. Based on this calculation result, the average number of nucleic acids having at least one of a full-length nucleotide sequence and a partial nucleotide sequence of rRNA or rDNA included in one cell can be calculated. The estimated number of nucleic acids having at least one of a full-length nucleotide sequence and a partial nucleotide sequence of rRNA or rDNA can be calculated by multiplying the counted cell number by the obtained average number of nucleic acids having at least one of a full-length nucleotide sequence and a partial nucleotide sequence of rRNA or rDNA.

It is applicable to perform an operation of controlling the cell cycles before producing the cell suspension. By preparing the cells uniformly to a state before replication occurs or a state after replication has occurred, it is possible to calculate the number of nucleic acids having at least one of a full-length nucleotide sequence and a partial nucleotide sequence of rRNA or rDNA based on the cell number more accurately.

It is applicable to calculate the degree of certainty (probability) for the estimated defined copy number. By calculating the degree of certainty (probability), it is possible to express and output the degree of certainty as a variance or a standard deviation based on these values. When adding up influences of a plurality of factors, it is possible use a square root of the sum of the squares of the standard deviation commonly used. For example, a correct answer percentage for the number of cells discharged, the number of DNA in a cell, and a landing ratio at which discharged cells land in wells can be used as the factors. A highly influential factor may be selected for calculation.

<<Liquid Droplet Landing Step>>

The liquid droplet landing step is a step of discharging the cell suspension in the form of liquid droplets to sequentially land the liquid droplets in wells of a plate.

A liquid droplet means a gathering of a liquid formed by a surface tension.

Discharging means making the cell suspension fly in the form of liquid droplets.

“Sequentially” means “in order”.

Landing means making liquid droplets reach the wells.

As a discharging unit, a unit configured to discharge the cell suspension in the form of liquid droplets (hereinafter may also be referred to as “discharging head”) can be suitably used.

Examples of the method for discharging the cell suspension in the form of liquid droplets include an on-demand method and a continuous method that are based on the inkjet method. Of these methods, in the case of the continuous method, there is a tendency that the dead volume of the cell suspension used is high, because of, for example, empty discharging until the discharging state becomes stable, adjustment of the amount of liquid droplets, and continued formation of liquid droplets even during transfer between the wells. In the present disclosure, in terms of cell number adjustment, it is applicable to suppress influence due to the dead volume. Hence, of the two methods, the on-demand method is more preferable.

Examples of the on-demand method include a plurality of known methods such as a pressure applying method of applying a pressure to a liquid to discharge the liquid, a thermal method of discharging a liquid by film boiling due to heating, and an electrostatic method of drawing liquid droplets by electrostatic attraction to form liquid droplets. Among these methods, the pressure applying method is preferable for the reason described below.

In the electrostatic method, there is a need for disposing an electrode in a manner to face a discharging unit that is configured to retain the cell suspension and form liquid droplets. In the method for producing a device, a plate for receiving liquid droplets is disposed at the facing position. Hence, it is applicable not to provide an electrode, in order to increase the degree of latitude in the plate configuration.

In the thermal method, there are a risk of local heating concentration that may affect the cells, which are a biomaterial, and a risk of kogation to the heater portion. Influences by heat depend on the components contained or the purpose for which the plate is used. Therefore, there is no need for flatly rejecting the thermal method. However, the pressure applying method is preferable because the pressure applying method has a lower risk of kogation to the heater portion than the thermal method.

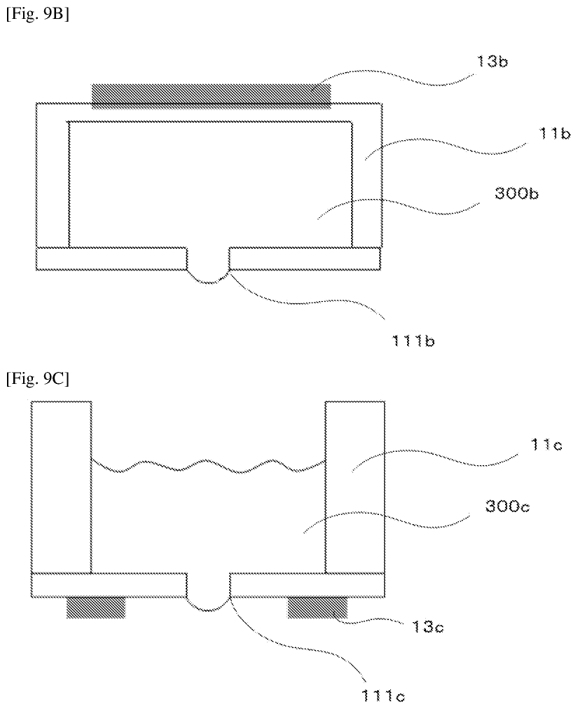

Examples of the pressure applying method include a method of applying a pressure to a liquid using a piezo element, and a method of applying a pressure using a valve such as an electromagnetic valve. The configuration example of a liquid droplet generating device usable for discharging liquid droplets of the cell suspension is illustrated in FIG. 9A to FIG. 9C.

FIG. 9A is an exemplary diagram illustrating an example of an electromagnetic valve-type discharging head. The electromagnetic valve-type discharging head includes an electric motor 13a, an electromagnetic valve 112, a liquid chamber 11a, a cell suspension 300a, and a nozzle 111a.

As the electromagnetic valve-type discharging head, for example, a dispenser of Tech Elan LLC can be suitably used.

FIG. 9B is an exemplary diagram illustrating an example of a piezo-type discharging head. The piezo-type discharging head includes a piezoelectric element 13b, a liquid chamber 11b, a cell suspension 300b, and a nozzle 111b.

As the piezo-type discharging head, for example, a single cell printer of Cytena GmbH can be suitably used.

Any of these discharging heads may be used. However, the pressure applying method by the electromagnetic valve is not capable of forming liquid droplets at a high speed repeatedly. Therefore, it is applicable to use the piezo method in order to increase the throughput of producing a plate. A piezo-type discharging head using a common piezoelectric element 13b may cause unevenness in the cell concentration due to settlement, or may have nozzle clogging.

Therefore, a more preferable configuration is the configuration illustrated in FIG. 9C. FIG. 9C is an exemplary diagram of a modified example of a piezo-type discharging head using the piezoelectric element illustrated in FIG. 9B. The discharging head of FIG. 9C includes a piezoelectric element 13c, a liquid chamber 11c, a cell suspension 300c, and a nozzle 111c.

In the discharging head of FIG. 9C, when a voltage is applied to the piezoelectric element 13c from an unillustrated control device, a compressive stress is applied in the horizontal direction of the drawing sheet. This can deform the membrane in the upward-downward direction of the drawing sheet.

Examples of any other method than the on-demand method include a continuous method for continuously forming liquid droplets. When pushing out liquid droplets from a nozzle by pressurization, the continuous method applies regular fluctuations using a piezoelectric element or a heater, to make it possible to continuously form minute liquid droplets. Further, the continuous method can select whether to land a flying liquid droplet into a well or to recover the liquid droplet in a recovery unit, by controlling the discharging direction of the liquid droplet with voltage application. Such a method is employed in a cell sorter or a flow cytometer. For example, a device named: CELL SORTER SH800Z of Sony Corporation can be used.

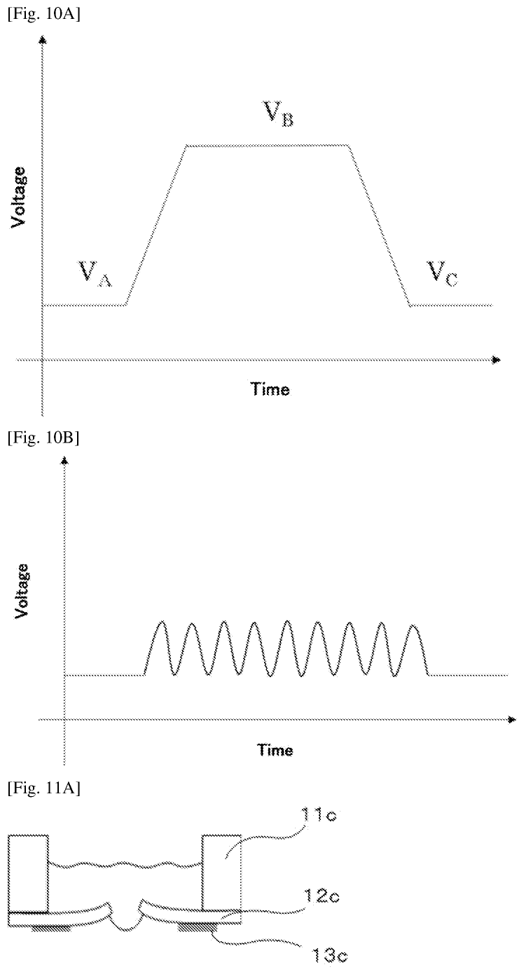

FIG. 10A is an exemplary graph plotting an example of a voltage applied to a piezoelectric element. FIG. 10B is an exemplary graph plotting another example of a voltage applied to a piezoelectric element. FIG. 10A plots a drive voltage for forming liquid droplets. Depending on the high or low level of the voltage (V_{A}, V_{B}, and V_{C}), it is possible to form liquid droplets. FIG. 10B plots a voltage for stirring the cell suspension without discharging liquid droplets.

During a period in which liquid droplets are not discharged, inputting a plurality of pulses that are not high enough to discharge liquid droplets enables the cell suspension in the liquid chamber to be stirred, making it possible to suppress occurrence of a concentration distribution due to settlement of the cells.

The liquid droplet forming operation of the discharging head that can be used in the present disclosure will be described below.

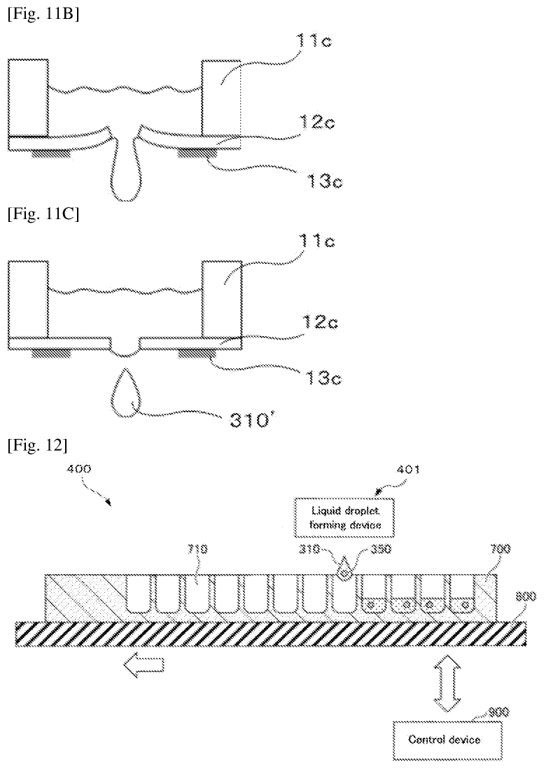

The discharging head can discharge liquid droplets with application of a pulsed voltage to the upper and lower electrodes formed on the piezoelectric element. FIG. 11A to FIG. 11C are exemplary diagrams illustrating liquid droplet states at the respective timings.

In FIG. 11A, first, upon application of a voltage to the piezoelectric element 13c, a membrane 12c abruptly deforms to cause a high pressure between the cell suspension retained in the liquid chamber 11c and the membrane 12c. This pressure pushes out a liquid droplet outward through the nozzle portion.

Next, as illustrated in FIG. 11B, for a period of time until when the pressure relaxes upward, the liquid is continuously pushed out through the nozzle portion, to grow the liquid droplet.

Finally, as illustrated in FIG. 11C, when the membrane 12c returns to the original state, the liquid pressure about the interface between the cell suspension and the membrane 12c lowers, to form a liquid droplet 310′.

In the method for producing a device, a plate in which wells are formed is secured on a movable stage, and by combination of driving of the stage with formation of liquid droplets from the discharging head, liquid droplets are sequentially landed in the concaves. A method of moving the plate along with moving the stage is described here. However, naturally, it is also possible to move the discharging head.

The plate is not particularly limited, and a plate that is commonly used in molecular biology fields and in which wells are formed can be used.

The number of wells in the plate is not particularly limited and may be appropriately selected depending on the intended purpose. The number of wells may be a single number or a plural number.

FIG. 12 is a schematic diagram illustrating an example of a dispensing device 400 configured to land liquid droplets sequentially into wells of a plate.

As illustrated in FIG. 12, the dispensing device 400 configured to land liquid droplets includes a liquid droplet forming device 401, a plate 700, a stage 800, and a control device 900.

In the dispensing device 400, the plate 700 is disposed over a movable stage 800. The plate 700 has a plurality of wells 710 (concaves) in which liquid droplets 310 discharged from a discharging head of the liquid droplet forming device 401 land. The control device 900 is configured to move the stage 800 and control the relative positional relationship between the discharging head of the liquid droplet forming device 401 and each well 710. This enables liquid droplets 310 containing fluorescent-stained cells 350 to be discharged sequentially into the wells 710 from the discharging head of the liquid droplet forming device 401.

The control device 900 may be configured to include, for example, a CPU, a ROM, a RAM, and a main memory. In this case, various functions of the control device 900 can be realized by a program recorded in, for example, the ROM being read out into the main memory and executed by the CPU. However, a part or the whole of the control device 900 may be realized only by hardware. Alternatively, the control device 900 may be configured with, for example, physically a plurality of devices.

When landing the cell suspension into the wells, it is applicable to land the liquid droplets to be discharged into the wells, in a manner that a plurality of levels is obtained.

A plurality of levels means a plurality of references serving as standards.

As the plurality of levels, it is applicable that a plurality of cells including a nucleic acid having at least one of a full-length nucleotide sequence and a partial nucleotide sequence of rRNA or rDNA have a predetermined concentration gradient in the wells. With a concentration gradient, the nucleic acid can be favorably used as a reagent for calibration curve. The plurality of levels can be controlled using values counted by a sensor.

As the plate, it is applicable to use, for example, a 1-well microtube, 8-series tubes, a 96-well plate, and a 384-well plate. When the number of wells is a plural number, it is possible to dispense the same number of cells into the wells of these plates, or it is also possible to dispense numbers of cells of different levels into the wells. There may be a well in which no cells are contained. Particularly, for producing a plate used for evaluating a real-time PCR device or digital PCR device configured to quantitatively evaluate an amount of nucleic acids, it is applicable to dispense numbers of nucleic acids of a plurality of levels. For example, it is conceivable to produce a plate into which cells (or nucleic acids) are dispensed at 7 levels, namely about 1 cell, 2 cells, 4 cells, 8 cells, 16 cells, 32 cells, and 64 cells. Using such a plate, it is possible to inspect, for example, quantitativity, linearity, and lower limit of evaluation of a real-time PCR device or digital PCR device.

<<Cell Number Counting Step>>

The cell number counting step is a step of counting the number of cells contained in the liquid droplets with a sensor after the liquid droplets are discharged and before the liquid droplets land in the wells.

A sensor means a device configured to, by utilizing some scientific principles, change mechanical, electromagnetic, thermal, acoustic, or chemical properties of natural phenomena or artificial products or spatial information/temporal information indicated by these properties into signals, which are a different medium easily handleable by humans or machines.

Counting Means Counting of Numbers.

The cell number counting step is not particularly limited and may be appropriately selected depending on the intended purpose, as long as the cell number counting step counts the number of cells contained in the liquid droplets with a sensor after the liquid droplets are discharged and before the liquid droplets land in the wells. The cell number counting step may include an operation for observing cells before discharging and an operation for counting cells after landing.

For counting the number of cells contained in the liquid droplets after the liquid droplets are discharged and before the liquid droplets land in the wells, it is applicable to observe cells in a liquid droplet at a timing at which the liquid droplet is at a position that is immediately above a well opening and at which the liquid droplet is predicted to enter the well in the plate without fail.

Examples of the method for observing cells in a liquid droplet include an optical detection method and an electric or magnetic detection method.

—Optical Detection Method—

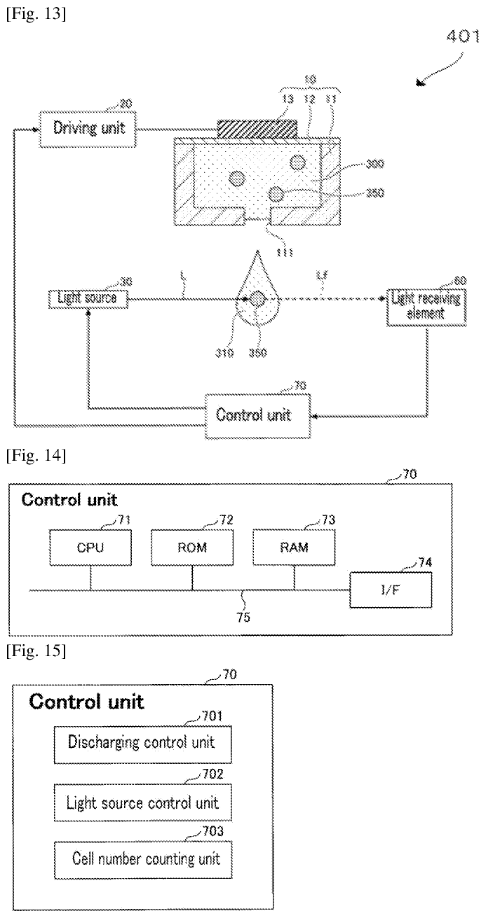

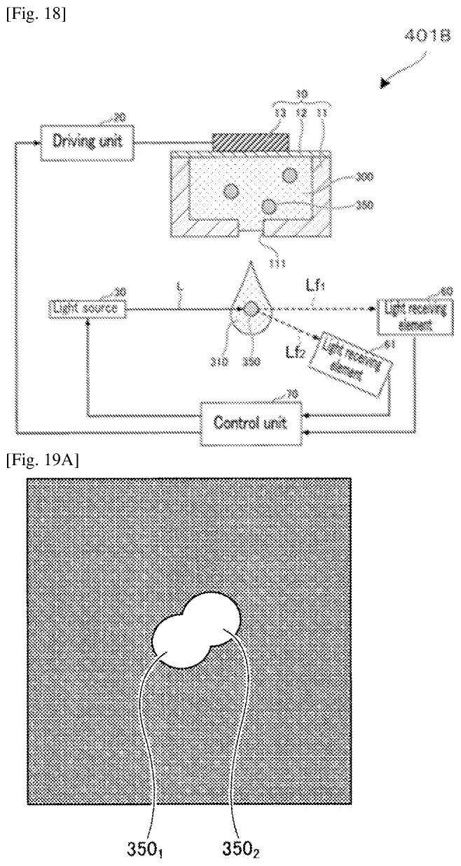

With reference to FIG. 13, FIG. 17, and FIG. 18, an optical detection method will be described below.

FIG. 13 is an exemplary diagram illustrating an example of a liquid droplet forming device 401. FIG. 17 and FIG. 18 are exemplary diagrams illustrating other examples of liquid droplet forming devices 401A and 401B. As illustrated in FIG. 13, the liquid droplet forming device 401 includes a discharging head (liquid droplet discharging unit) 10, a driving unit 20, a light source 30, a light receiving element 60, and a control unit 70.

In FIG. 13, a liquid obtained by dispersing cells in a predetermined solution after fluorescently staining the cells with a specific pigment is used as the cell suspension. Cells are counted by irradiating the liquid droplets formed by the discharging head with light having a specific wavelength and emitted from the light source and detecting fluorescence emitted by the cells with the light receiving element. Here, autofluorescence emitted by molecules originally contained in the cells may be utilized, in addition to the method of staining the cells with a fluorescent pigment. Alternatively, genes for producing fluorescent proteins (for example, GFP (Green Fluorescent Proteins)) may be previously introduced into the cells, in order that the cells may emit fluorescence.

Irradiation of light means application of light.

The discharging head 10 includes a liquid chamber 11, a membrane 12, and a driving element 13 and can discharge a cell suspension 300 suspending fluorescent-stained cells 350 in the form of liquid droplets.

The liquid chamber 11 is a liquid retaining portion configured to retain the cell suspension 300 suspending the fluorescent-stained cells 350. A nozzle 111, which is a through hole, is formed in the lower surface of the liquid chamber 11. The liquid chamber 11 may be formed of, for example, a metal, silicon, or a ceramic. Examples of the fluorescent-stained cells 350 include inorganic particles and organic polymer particles stained with a fluorescent pigment.

The membrane 12 is a film-shaped member secured on the upper end portion of the liquid chamber 11. The planar shape of the membrane 12 may be, for example, a circular shape, but may also be, for example, an elliptic shape or a quadrangular shape.

The driving element 13 is provided on the upper surface of the membrane 12. The shape of the driving element 13 may be designed to match the shape of the membrane 12. For example, when the planar shape of the membrane 12 is a circular shape, it is applicable to provide a circular driving element 13.

The membrane 12 can be vibrated by supplying a driving signal to the driving element 13 from a driving unit 20. The vibration of the membrane 12 can cause a liquid droplet 310 containing the fluorescent-stained cells 350 to be discharged through the nozzle 111.

When a piezoelectric element is used as the driving element 13, for example, the driving element 13 may have a structure obtained by providing the upper surface and the lower surface of the piezoelectric material with electrodes across which a voltage is to be applied. In this case, when the driving unit 20 applies a voltage across the upper and lower electrodes of the piezoelectric element, a compressive stress is applied in the horizontal direction of the drawing sheet, making it possible for the membrane 12 to vibrate in the upward-downward direction of the drawing sheet. As the piezoelectric material, for example, lead zirconate titanate (PZT) may be used. In addition, various piezoelectric materials can be used, such as bismuth iron oxide, metal niobate, barium titanate, or materials obtained by adding metals or different oxides to these materials.

The light source 30 is configured to irradiate a flying liquid droplet 310 with light L. A flying state means a state from when the liquid droplet 310 is discharged from a liquid droplet discharging unit 10 until when the liquid droplet 310 lands on the landing target. A flying liquid droplet 310 has an approximately spherical shape at the position at which the liquid droplet 310 is irradiated with the light L. The beam shape of the light L is an approximately circular shape.

It is applicable that the beam diameter of the light L be from about 10 times through 100 times as great as the diameter of the liquid droplet 310. This is for ensuring that the liquid droplet 310 is irradiated with the light L from the light source 30 without fail even when the position of the liquid droplet 310 fluctuates.

However, it is not preferable if the beam diameter of the light L is much greater than 100 times as great as the diameter of the liquid droplet 310. This is because the energy density of the light with which the liquid droplet 310 is irradiated is reduced, to lower the light volume of fluorescence Lf to be emitted upon the light L serving as excitation light, making it difficult for the light receiving element 60 to detect the fluorescence Lf.

It is applicable that the light L emitted by the light source 30 be pulse light. It is applicable to use, for example, a solid-state laser, a semiconductor laser, and a dye laser. When the light L is pulse light, the pulse width is preferably 10 microseconds or less and preferably 1 microsecond or less. The energy per unit pulse is preferably roughly 0.1 microjoules or higher and preferably 1 microjoule or higher, although significantly depending on the optical system such as presence or absence of light condensation.

The light receiving element 60 is configured to receive fluorescence Lf emitted by the fluorescent-stained cell 350 upon absorption of the light L as excitation light, when the fluorescent-stained cell 350 is contained in a flying liquid droplet 310. Because the fluorescence Lf is emitted to all directions from the fluorescent-stained cell 350, the light receiving element 60 can be disposed at an arbitrary position at which the fluorescence Lf is receivable. Here, in order to improve contrast, it is applicable to dispose the light receiving element 60 at a position at which direct incidence of the light L emitted by the light source 30 to the light receiving element 60 does not occur.

The light receiving element 60 is not particularly limited and may be appropriately selected depending on the intended purpose as long as the light receiving element 60 is an element capable of receiving the fluorescence Lf emitted by the fluorescent-stained cell 350. An optical sensor configured to receive fluorescence from a cell in a liquid droplet when the liquid droplet is irradiated with light having a specific wavelength is preferable. Examples of the light receiving element 60 include one-dimensional elements such as a photodiode and a photosensor. When high-sensitivity measurement is needed, it is applicable to use a photomultiplier tube and an Avalanche photodiode. As the light receiving element 60, two-dimensional elements such as a CCD (Charge Coupled Device), a CMOS (Complementary Metal Oxide Semiconductor), and a gate CCD may be used.

The fluorescence Lf emitted by the fluorescent-stained cell 350 is weaker than the light L emitted by the light source 30. Therefore, a filter configured to attenuate the wavelength range of the light L may be installed at a preceding stage (light receiving surface side) of the light receiving element 60. This enables the light receiving element 60 to obtain an extremely highly contrastive image of the fluorescent-stained cell 350. As the filter, for example, a notch filter configured to attenuate a specific wavelength range including the wavelength of the light L may be used.

As described above, it is applicable that the light L emitted by the light source 30 be pulse light. The light L emitted by the light source 30 may be continuously oscillating light. In this case, it is applicable to control the light receiving element 60 to be capable of receiving light at a timing at which a flying liquid droplet 310 is irradiated with the continuously oscillating light, to make the light receiving element 60 receive the fluorescence Lf.

The control unit 70 has a function of controlling the driving unit 20 and the light source 30. The control unit 70 also has a function of obtaining information that is based on the light volume received by the light receiving element 60 and counting the number of fluorescent-stained cells 350 contained in the liquid droplet 310 (the case where the number is zero is also included). With reference to FIG. 14 to FIG. 16, an operation of the liquid droplet forming device 401 including an operation of the control unit 70 will be described below.

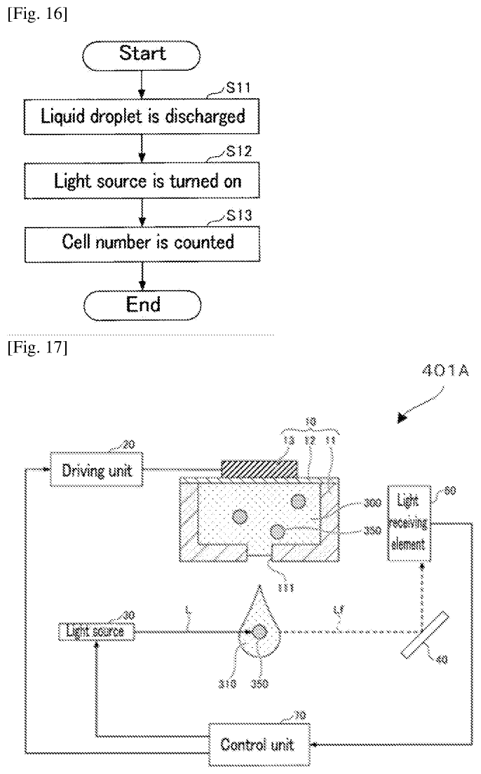

FIG. 14 is a diagram illustrating hardware blocks of the control unit of the liquid droplet forming device of FIG. 13. FIG. 15 is a diagram illustrating functional blocks of the control unit of the liquid droplet forming device of FIG. 13. FIG. 16 is a flowchart illustrating an example of the operation of the liquid droplet forming device.

As illustrated in FIG. 14, the control unit 70 includes a CPU 71, a ROM 72, a RAM 73, an I/F 74, and a bus line 75. The CPU 71, the ROM 72, the RAM 73, and the I/F 74 are coupled to one another via the bus line 75.

The CPU 71 is configured to control various functions of the control unit 70. The ROM 72 serving as a memory unit is configured to store programs to be executed by the CPU 71 for controlling the various functions of the control unit 70 and various information. The RAM 73 serving as a memory unit is configured to be used as, for example, the work area of the CPU 71. The RAM 73 is also configured to be capable of storing predetermined information for a temporary period of time. The I/F 74 is an interface configured to couple the liquid droplet forming device 401 to, for example, another device. The liquid droplet forming device 401 may be coupled to, for example, an external network via the I/F 74.

As illustrated in FIG. 15, the control unit 70 includes a discharging control unit 701, a light source control unit 702, and a cell number counting unit (cell number sensing unit) 703 as functional blocks.

With reference to FIG. 15 and FIG. 16, cell number (particle number) counting by the liquid droplet forming device 401 will be described.

In the step S11, the discharging control unit 701 of the control unit 70 outputs an instruction for discharging to the driving unit 20. Upon reception of the instruction for discharging from the discharging control unit 701, the driving unit 20 supplies a driving signal to the driving element 13 to vibrate the membrane 12. The vibration of the membrane 12 causes a liquid droplet 310 containing a fluorescent-stained cell 350 to be discharged through the nozzle 111.

Next, in the step S12, the light source control unit 702 of the control unit 70 outputs an instruction for lighting to the light source 30 in synchronization with the discharging of the liquid droplet 310 (in synchronization with a driving signal supplied by the driving unit 20 to the liquid droplet discharging unit 10). In accordance with this instruction, the light source 30 is turned on to irradiate the flying liquid droplet 310 with the light L.

Here, the light is emitted by the light source 30, not in synchronization with discharging of the liquid droplet 310 by the liquid droplet discharging unit 10 (supplying of the driving signal to the liquid droplet discharging unit 10 by the driving unit 20), but in synchronization with the timing at which the liquid droplet 310 has come flying to a predetermined position in order for the liquid droplet 310 to be irradiated with the light L. That is, the light source control unit 702 controls the light source 30 to emit light at a predetermined period of time of delay from the discharging of the liquid droplet 310 by the liquid droplet discharging unit 10 (from the driving signal supplied by the driving unit 20 to the liquid droplet discharging unit 10).

For example, the speed v of the liquid droplet 310 to be discharged when the driving signal is supplied to the liquid droplet discharging unit 10 may be measured beforehand. Based on the measured speed v, the time t taken from when the liquid droplet 310 is discharged until when the liquid droplet 310 reaches the predetermined position may be calculated, in order that the timing of light irradiation by the light source 30 may be delayed from the timing at which the driving signal is supplied to the liquid droplet discharging unit 10 by the period of time of t. This enables a good control on light emission, and can ensure that the liquid droplet 310 is irradiated with the light from the light source 30 without fail.

Next, in the step S13, the cell number counting unit 703 of the control unit 70 counts the number of fluorescent-stained cells 350 contained in the liquid droplet 310 (the case where the number is zero is also included) based on information from the light receiving element 60. The information from the light receiving element 60 indicates the luminance (light volume) and the area value of the fluorescent-stained cell 350.

The cell number counting unit 703 can count the number of fluorescent-stained cells 350 by, for example, comparing the light volume received by the light receiving element 60 with a predetermined threshold. In this case, a one-dimensional element may be used or a two-dimensional element may be used as the light receiving element 60.

When a two-dimensional element is used as the light receiving element 60, the cell number counting unit 703 may use a method of performing image processing for calculating the luminance or the area of the fluorescent-stained cell 350 based on a two-dimensional image obtained from the light receiving element 60. In this case, the cell number counting unit 703 can count the number of fluorescent-stained cells 350 by calculating the luminance or the area value of the fluorescent-stained cell 350 by image processing and comparing the calculated luminance or area value with a predetermined threshold.

The fluorescent-stained cell 350 may be a cell or a stained cell. A stained cell means a cell stained with a fluorescent pigment or a cell that can express a fluorescent protein.

The fluorescent pigment for the stained cell is not particularly limited and may be appropriately selected depending on the intended purpose. Examples of the fluorescent pigment include fluoresceins, rhodamines, coumarins, pyrenes, cyanines, and azo pigments. One of these fluorescent pigments may be used alone or two or more of these fluorescent pigments may be used in combination. Among these fluorescent pigments, eosin, Evans blue, trypan blue, rhodamine 6G, rhodamine B, and Rhodamine 123 are more preferable.

Examples of the fluorescent protein include Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, mPlum, PS-CFP, Dendra2, Kaede, EosFP, and KikumeGR. One of these fluorescent proteins may be used alone or two or more of these fluorescent proteins may be used in combination.

In this way, in the liquid droplet forming device 401, the driving unit 20 supplies a driving signal to the liquid droplet discharging unit 10 retaining the cell suspension 300 suspending fluorescent-stained cells 350 to cause the liquid droplet discharging unit 10 to discharge a liquid droplet 310 containing the fluorescent-stained cell 350, and the flying liquid droplet 310 is irradiated with the light L from the light source 30. Then, the fluorescent-stained cell 350 contained in the flying liquid droplet 310 emits the fluorescence Lf upon the light L serving as excitation light, and the light receiving element 60 receives the fluorescence Lf. Then, the cell number counting unit 703 counts the number of fluorescent-stained cells 350 contained in the flying liquid droplet 310, based on information from the light receiving element 60.

That is, the liquid droplet forming device 401 is configured for on-the-spot actual observation of the number of fluorescent-stained cells 350 contained in the flying liquid droplet 310. This can realize a better accuracy than hitherto obtained, in counting the number of fluorescent-stained cells 350. Moreover, because the fluorescent-stained cell 350 contained in the flying liquid droplet 310 is irradiated with the light L and emits the fluorescence Lf that is to be received by the light receiving element 60, an image of the fluorescent-stained cell 350 can be obtained with a high contrast, and the frequency of occurrence of erroneous counting of the number of fluorescent-stained cells 350 can be reduced.

FIG. 17 is an exemplary diagram illustrating a modified example of the liquid droplet forming device 401 of FIG. 13. As illustrated in FIG. 17, a liquid droplet forming device 401A is different from the liquid droplet forming device 401 (see FIG. 13) in that a mirror 40 is arranged at the preceding stage of the light receiving element 60. Description about components that are the same as in the embodiment already described may be skipped.

In the liquid droplet forming device 401A, arranging the mirror 40 at the perceiving stage of the light receiving element 60 can improve the degree of latitude in the layout of the light receiving element 60.

For example, in the layout of FIG. 13, when a nozzle 111 and a landing target are brought close to each other, there is a risk of occurrence of interference between the landing target and the optical system (particularly, the light receiving element 60) of the liquid droplet forming device 401. With the layout of FIG. 17, occurrence of interference can be avoided.

That is, by changing the layout of the light receiving element 60 as illustrated in FIG. 17, it is possible to reduce the distance (gap) between the landing target on which a liquid droplet 310 is landed and the nozzle 111 and suppress landing on a wrong position. As a result, the dispensing accuracy can be improved.

FIG. 18 is an exemplary diagram illustrating another modified example of the liquid droplet forming device 401 of FIG. 13. As illustrated in FIG. 18, a liquid droplet forming device 401B is different from the liquid droplet forming device 401 (see FIG. 13) in that a light receiving element 61 configured to receive fluorescence Lf₂ emitted by the fluorescent-stained cell 350 is provided in addition to the light receiving element 60 configured to receive fluorescence Lf₁ emitted by the fluorescent-stained cell 350. Description about components that are the same as in the embodiment already described may be skipped.

The fluorescences Lf₁ and Lf₂ represent parts of fluorescence emitted to all directions from the fluorescent-stained cell 350. The light receiving elements 60 and 61 can be disposed at arbitrary positions at which the fluorescence emitted to different directions by the fluorescent-stained cell 350 is receivable. Three or more light receiving elements may be disposed at positions at which the fluorescence emitted to different directions by the fluorescent-stained cell 350 is receivable. The light receiving elements may have the same specifications or different specifications.

With one light receiving element, when a plurality of fluorescent-stained cells 350 are contained in a flying liquid droplet 310, there is a risk that the cell number counting unit 703 may erroneously count the number of fluorescent-stained cells 350 contained in the liquid droplet 310 (a risk that a counting error may occur) because the fluorescent-stained cells 350 may overlap each other.

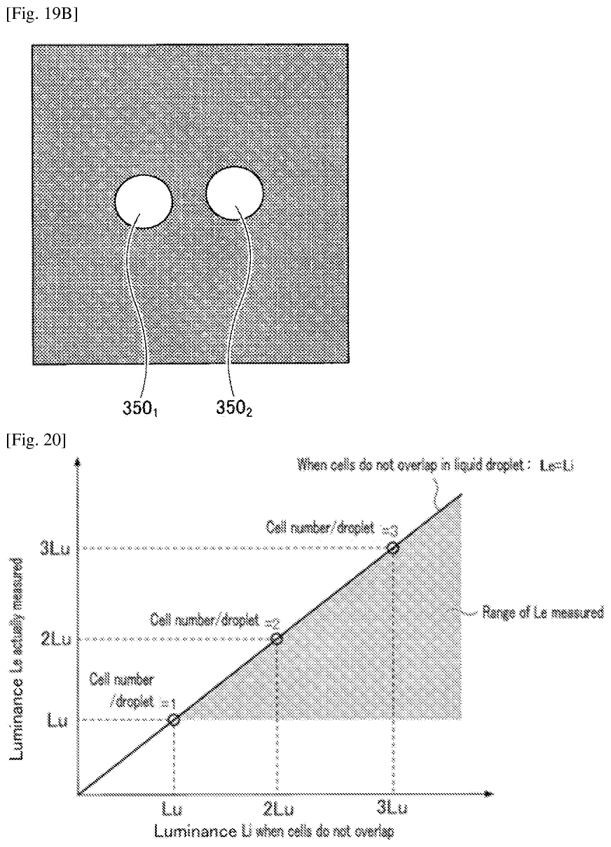

FIG. 19A and FIG. 19B are diagrams illustrating a case where two fluorescent-stained cells are contained in a flying liquid droplet. For example, as illustrated in FIG. 19A, there may be a case where fluorescent-stained cells 350₁ and 350₂ overlap each other, or as illustrated in FIG. 19B, there may be a case where the fluorescent-stained cells 350₁ and 350₂ do not overlap each other. By providing two or more light receiving elements, it is possible to reduce the influence of overlap of the fluorescent-stained cells.

As described above, the cell number counting unit 703 can count the number of fluorescent particles, by calculating the luminance or the area value of fluorescent particles by image processing and comparing the calculated luminance or area value with a predetermined threshold.

When two or more light receiving elements are installed, it is possible to suppress occurrence of a counting error, by adopting the data indicating the maximum value among the luminance values or area values obtained from these light receiving elements. This will be described in more detail with reference to FIG. 20.

FIG. 20 is a graph plotting an example of a relationship between a luminance Li when particles do not overlap each other and a luminance Le actually measured. As plotted in FIG. 20, when particles in the liquid droplet do not overlap each other, Le is equal to Li. For example, in the case where the luminance of one cell is assumed to be Lu, Le is equal to Lu when the number of cells per droplet is 1, and Le is equal to nLu when the number of cells per droplet is n (n: natural number).

However, actually, when n is 2 or greater, because particles may overlap each other, the luminance to be actually measured is Lu≤Le≤nLu (the half-tone dot meshed portion in FIG. 20). Hence, when the number of cells per droplet is n, the threshold may be set to, for example, (nLu−Lu/2)≤threshold<(nLu+Lu/2). When a plurality of light receiving elements are installed, it is possible to suppress occurrence of a counting error, by adopting the maximum value among the data obtained from these light receiving elements. An area value may be used instead of luminance.

When a plurality of light receiving elements are installed, the number of particles may be determined according to an algorithm for estimating the number of cells based on a plurality of shape data to be obtained.

As can be understood, with the plurality of light receiving elements configured to receive fluorescence emitted to different directions by the fluorescent-stained cell 350, the liquid droplet forming device 401B can further reduce the frequency of occurrence of erroneous counting of the number of fluorescent-stained cells 350.

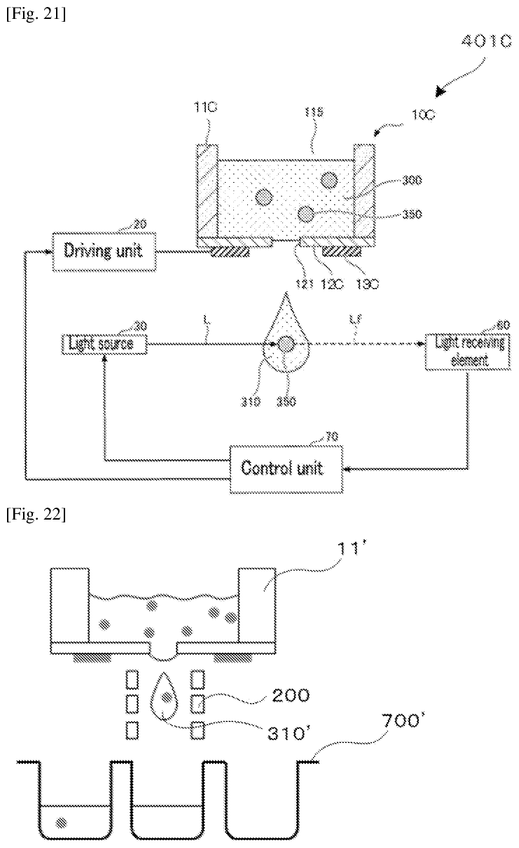

FIG. 21 is an exemplary diagram illustrating another modified example of the liquid droplet forming device 401 of FIG. 13. As illustrated in FIG. 21, a liquid droplet forming device 401C is different from the liquid droplet forming device 401 (see FIG. 13) in that a liquid droplet discharging unit 10C is provided instead of the liquid droplet discharging unit 10. Description about components that are the same as in the embodiment already described may be skipped.

The liquid droplet discharging unit 10C includes a liquid chamber 11C, a membrane 12C, and a driving element 13C. At the top, the liquid chamber 11C has an atmospherically exposed portion 115 configured to expose the interior of the liquid chamber 11C to the atmosphere, and bubbles mixed in the cell suspension 300 can be evacuated through the atmospherically exposed portion 115.

The membrane 12C is a film-shaped member secured at the lower end of the liquid chamber 11C. A nozzle 121, which is a through hole, is formed in approximately the center of the membrane 12C, and the vibration of the membrane 12C causes the cell suspension 300 retained in the liquid chamber 11C to be discharged through the nozzle 121 in the form of a liquid droplet 310. Because the liquid droplet 310 is formed by the inertia of the vibration of the membrane 12C, it is possible to discharge the cell suspension 300 even when the cell suspension 300 has a high surface tension (a high viscosity). The planar shape of the membrane 12C may be, for example, a circular shape, but may also be, for example, an elliptic shape or a quadrangular shape.

The material of the membrane 12C is not particularly limited. However, if the material of the membrane 12C is extremely flexible, the membrane 12C easily undergo vibration and is not easily able to stop vibration immediately when there is no need for discharging. Therefore, a material having a certain degree of hardness is preferable. As the material of the membrane 12C, for example, a metal material, a ceramic material, and a polymeric material having a certain degree of hardness can be used.

Particularly, when a cell is used as the fluorescent-stained cell 350, the material of the membrane is preferably a material having a low adhesiveness with the cell or proteins. Generally, adhesiveness of cells is said to be dependent on the contact angle of the material with respect to water. When the material has a high hydrophilicity or a high hydrophobicity, the material has a low adhesiveness with cells. As the material having a high hydrophilicity, various metal materials and ceramics (metal oxides) can be used. As the material having a high hydrophobicity, for example, fluororesins can be used.

Other examples of such materials include stainless steel, nickel, and aluminum, and silicon dioxide, alumina, and zirconia. In addition, it is conceivable to reduce cell adhesiveness by coating the surface of the material. For example, it is possible to coat the surface of the material with the metal or metal oxide materials described above, or coat the surface of the material with a synthetic phospholipid polymer mimicking a cellular membrane (e.g., LIPIDURE of NOF Corporation).

It is applicable that the nozzle 121 be formed as a through hole having a substantially perfect circle shape in approximately the center of the membrane 12C. In this case, the diameter of the nozzle 121 is not particularly limited but is preferably twice or more greater than the size of the fluorescent-stained cell 350 in order to prevent the nozzle 121 from being clogged with the fluorescent-stained cell 350. When the fluorescent-stained cell 350 is, for example, an animal cell, particularly, a human cell, the diameter of the nozzle 121 is in the following order of preference (from lowest to highest): 10 micrometers or greater, and 100 micrometers or greater in conformity with the cell used, because a human cell typically has a size of about from 5 micrometers through 50 micrometers.

On the other hand, when a liquid droplet is extremely large, it is difficult to achieve an object of forming a minute liquid droplet. Therefore, the diameter of the nozzle 121 is preferably 200 micrometers or less. That is, in the liquid droplet discharging unit 10C, the diameter of the nozzle 121 is typically in the range of from 10 micrometers through 200 micrometers.

The driving element 13C is formed on the lower surface of the membrane 12C. The shape of the driving element 13C can be designed to match the shape of the membrane 12C. For example, when the planar shape of the membrane 12C is a circular shape, it is applicable to form a driving element 13C having an annular (ring-like) planar shape around the nozzle 121. The driving method for driving the driving element 13C may be the same as the driving method for driving the driving element 13.

The driving unit 20 can selectively (for example, alternately) apply to the driving element 13C, a discharging waveform for vibrating the membrane 12C to form a liquid droplet 310 and a stirring waveform for vibrating the membrane 12C to an extent until which a liquid droplet 310 is not formed.

For example, the discharging waveform and the stirring waveform may both be rectangular waves, and the driving voltage for the stirring waveform may be set lower than the driving voltage for the discharging waveform. This makes it possible for a liquid droplet 310 not to be formed by application of the stirring waveform. That is, it is possible to control the vibration state (degree of vibration) of the membrane 12C depending on whether the driving voltage is high or low.

In the liquid droplet discharging unit 10C, the driving element 13C is formed on the lower surface of the membrane 12C. Therefore, when the membrane 12 is vibrated by means of the driving element 13C, a flow can be generated in a direction from the lower portion to the upper portion in the liquid chamber 11C.

Here, the fluorescent-stained cells 350 move upward from lower positions, to generate a convection current in the liquid chamber 11C to stir the cell suspension 300 containing the fluorescent-stained cells 350. The flow from the lower portion to the upper portion in the liquid chamber 11C disperses the settled, aggregated fluorescent-stained cells 350 uniformly in the liquid chamber 11C.

That is, by applying the discharging waveform to the driving element 13C and controlling the vibration state of the membrane 12C, the driving unit 20 can cause the cell suspension 300 retained in the liquid chamber 11C to be discharged through the nozzle 121 in the form of a liquid droplet 310. Further, by applying the stirring waveform to the driving element 13C and controlling the vibration state of the membrane 12C, the driving unit 20 can stir the cell suspension 300 retained in the liquid chamber 11C. During stirring, no liquid droplet 310 is discharged through the nozzle 121.

In this way, stirring the cell suspension 300 while no liquid droplet 310 is being formed can prevent settlement and aggregation of the fluorescent-stained cells 350 over the membrane 12C and can disperse the fluorescent-stained cells 350 in the cell suspension 300 without unevenness. This can suppress clogging of the nozzle 121 and variation in the number of fluorescent-stained cells 350 in the liquid droplets 310 to be discharged. This makes it possible to stably discharge the cell suspension 300 containing the fluorescent-stained cells 350 in the form of liquid droplets 310 continuously for a long time.

In the liquid droplet forming device 401C, bubbles may mix in the cell suspension 300 in the liquid chamber 11C. Also in this case, with the atmospherically exposed portion 115 provided at the top of the liquid chamber 11C, the liquid droplet forming device 401C can be evacuated of the bubbles mixed in the cell suspension 300 to the outside air through the atmospherically exposed portion 115. This enables continuous, stable formation of liquid droplets 310 without a need for disposing of a large amount of the liquid for bubble evacuation.

That is, the discharging state is affected when mixed bubbles are present at a position near the nozzle 121 or when many mixed bubbles are present over the membrane 12C. Therefore, in order to perform stable formation of liquid droplets for a long time, there is a need for eliminating the mixed bubbles. Typically, mixed bubbles present over the membrane 12C move upward autonomously or by vibration of the membrane 12C. Because the liquid chamber 11C is provided with the atmospherically exposed portion 115, the mixed bubbles can be evacuated through the atmospherically exposed portion 115. This makes it possible to prevent occurrence of empty discharging even when bubbles mix in the liquid chamber 11C, enabling continuous, stable formation of liquid droplets 310.

At a timing at which a liquid droplet is not being formed, the membrane 12C may be vibrated to an extent until which a liquid droplet is not formed, in order to positively move the bubbles upward in the liquid chamber 11C.

—Electric or Magnetic Detection Method—

In the case of the electric or magnetic detection method, as illustrated in FIG. 22, a coil 200 configured to count the number of cells is installed as a sensor immediately below a discharging head configured to discharge the cell suspension onto a plate 700′ from a liquid chamber 11′ in the form of a liquid droplet 310′. Cells are coated with magnetic beads that are modified with a specific protein and can adhere to the cells. Therefore, when the cells to which magnetic beads adhere pass through the coil, an induced current is generated to enable detection of presence or absence of the cells in the flying liquid droplet. Generally, cells have proteins specific to the cells on the surfaces of the cells. Modification of magnetic beads with antibodies that can adhere to the proteins enables adhesion of the magnetic beads to the cells. As such magnetic beads, a ready-made product can be used. For example, DYNABEADS (registered trademark) of Veritas Corporation can be used.

<Operation for Observing Cells Before Discharging>

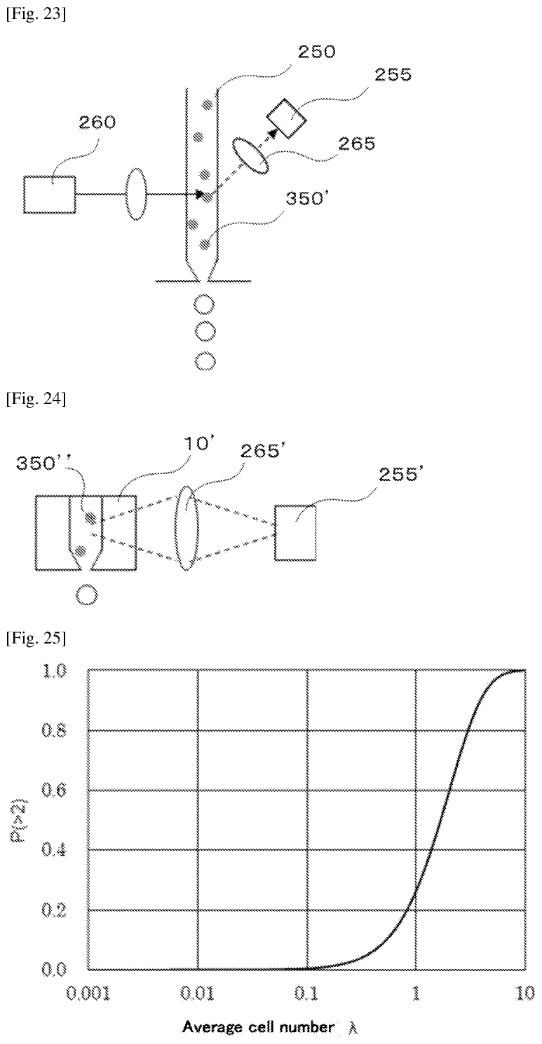

The operation for observing cells before discharging may be performed by, for example, a method for counting cells 350′ that have passed through a micro-flow path 250 illustrated in FIG. 23 or a method for capturing an image of a portion near a nozzle portion of a discharging head illustrated in FIG. 24. The method of FIG. 23 is a method used in a cell sorter device, and, for example, CELL SORTER SH800Z of Sony Corporation can be used. In FIG. 23, a light source 260 emits laser light into the micro-flow path 250, and a detector 255 detects scattered light or fluorescence through a condenser lens 265. This enables discrimination of presence or absence of cells or the kind of the cells, while a liquid droplet is being formed. Based on the number of cells that have passed through the micro-flow path 250, this method enables estimation of the number of cells that have landed in a predetermined well.

As the discharging head 10′ illustrated in FIG. 24, a single cell printer of Cytena GmbH can be used. In FIG. 24, it is possible to estimate the number of cells that have landed in a predetermined well, by capturing an image of the portion near the nozzle portion with an image capturing unit 255′ through a lens 265′ before discharging and estimating based on the captured image that cells 350″ present near the nozzle portion have been discharged, or by estimating the number of cells that are considered to have been discharged based on a difference between images captured before and after discharging. The method of FIG. 24 is more preferable because the method enables on-demand liquid droplet formation, whereas the method of FIG. 23 for counting cells that have passed through the micro-flow path generates liquid droplets continuously.

<Operation for Counting Cells after Landing>

The operation for counting cells after landing may be performed by a method for detecting fluorescent-stained cells by observing the wells in the plate with, for example, a fluorescence microscope. This method is described in, for example, Sangjun et al., PLoS One, Volume 6(3), e17455.

Methods for observing cells before discharging a liquid droplet or after landing have the problems described below. Depending on the kind of the plate to be produced, it is the most applicable to observe cells in a liquid droplet that is being discharged. In the method for observing cells before discharging, the number of cells that are considered to have landed is counted based on the number of cells that have passed through a flow path and image observation before discharging (and after discharging). Therefore, it is not confirmed whether the cells have actually been discharged, and an unexpected error may occur. For example, there may be a case where because the nozzle portion is stained, a liquid droplet is not discharged appropriately but adheres to the nozzle plate, thus failing to make the cells in the liquid droplet land. Moreover, there may occur a problem that the cells stay behind in a narrow region of the nozzle portion, or a discharging operation causes the cells to move beyond assumption and go outside the range of observation.

The method for detecting cells on the plate after landing also have problems. First, there is a need for preparing a plate that can be observed with a microscope. As a plate that can be observed, it is common to use a plate having a transparent, flat bottom surface, particularly a plate having a bottom surface formed of glass. However, there is a problem that such a special plate is incompatible with use of ordinary wells. Further, when the number of cells is large, such as some tens of cells, there is a problem that correct counting is impossible because the cells may overlap with each other. Accordingly, it is applicable to perform the operation for observing cells before discharging and the operation for counting cells after landing, in addition to counting the number of cells contained in a liquid droplet with a sensor and a particle number (cell number) counting unit after the liquid droplet is discharged and before the liquid droplet lands in a well.

As the light receiving element, a light receiving element including one or a small number of light receiving portion(s), such as a photodiode, an Avalanche photodiode, and a photomultiplier tube may be used. In addition, a two-dimensional sensor including light receiving elements in a two-dimensional array formation, such as a CCD (Charge Coupled Device), a CMOS (Complementary Metal Oxide Semiconductor), and a gate CCD may be used.

When using a light receiving element including one or a small number of light receiving portion(s), it is conceivable to determine the number of cells contained, based on the fluorescence intensity, using a calibration curve prepared beforehand. Here, binary detection of whether cells are present or absent in a flying liquid droplet is common. When the cell suspension is discharged in a state that the cell concentration is so sufficiently low that almost only 1 or 0 cell(s) will be contained in a liquid droplet, sufficiently accurate counting is available by the binary detection. On the premise that cells are randomly distributed in the cell suspension, the cell number in a flying liquid droplet is considered to conform to a Poisson distribution, and the probability P (>2) at which two or more cells are contained in a liquid droplet is represented by a formula (1) below. FIG. 25 is a graph plotting a relationship between the probability P (>2) and an average cell number. Here, λ is a value representing an average cell number in a liquid droplet and obtained by multiplying the cell concentration in the cell suspension by the volume of a liquid droplet discharged.

P(>2)=1−(1−λ)×e^{-λ} formula (1)

When performing cell number counting by binary detection, in order to ensure accuracy, it is applicable that the probability P (>2) be a sufficiently low value, and that λ satisfy: λ<0.15, at which the probability P (>2) is 1% or lower. The light source is not particularly limited and may be appropriately selected depending on the intended purpose, as long as the light source can excite fluorescence from cells. It is possible to use, for example, an ordinary lamp such as a mercury lamp and a halogen lamp to which a filter is applied for emission of a specific wavelength, a LED (Light Emitting Diode), and a laser. However, particularly when forming a minute liquid droplet of 1 nL or less, there is a need for irradiating a small region with a high light intensity. Therefore, use of a laser is preferable. As a laser light source, various commonly known lasers such as a solid-state laser, a gas laser, and a semiconductor laser can be used. The excitation light source may be a light source that is configured to continuously irradiate a region through which a liquid droplet passes or may be a light source that is configured for pulsed irradiation in synchronization with discharging of a liquid droplet at a timing delayed by a predetermined period of time from the operation for discharging the liquid droplet.

<<<Step of Calculating Degrees of Certainty of Estimated Numbers of Nucleic Acids in Cell Suspension Producing Step, Liquid Droplet Landing Step, and Cell Number Counting Step>>>

The step of calculating degrees of certainty of estimated numbers of nucleic acids in the cell suspension producing step, the liquid droplet landing step, and the cell number counting step is a step of calculating the degree of certainty in each of the cell suspension producing step, the liquid droplet landing step, and the cell number counting step.

The degree of certainty of an estimated number of nucleic acids can be calculated in the same manner as calculating the degree of certainty in the cell suspension producing step.

The timing at which the degrees of certainty are calculated may be collectively in the next step to the cell number counting step, or may be at the end of each of the cell suspension producing step, the liquid droplet landing step, and the cell number counting step in order for the degrees of certainty to be summed in the next step to the cell number counting step. In other words, the degrees of certainty in these steps need only to be calculated at arbitrary timings by the time when summing is performed.

<<Outputting Step>>

The outputting step is a step of outputting a counted value of the number of cells contained in the cell suspension that has landed in a well, counted by a particle number counting unit based on a detection result measured by a sensor.

The counted value means a number of cells contained in the well, calculated by the particle number counting unit based on the detection result measured by the sensor.

Outputting means sending a value counted by a device such as a motor, communication equipment, and a calculator upon reception of an input to an external server serving as a count result memory unit in the form of electronic information, or printing the counted value as a printed matter.

In the outputting step, an observed value or an estimated value obtained by observing or estimating the number of cells or the number of nucleic acids in each well of a plate during production of the plate is output to an external memory unit.

Outputting may be performed at the same time as the cell number counting step, or may be performed after the cell number counting step.

<<Recording Step>>

The recording step is a step of recording the observed value or the estimated value output in the outputting step.

The recording step can be suitably performed by a recording unit.

Recording may be performed at the same time as the outputting step, or may be performed after the outputting step.

Recording means not only supplying information to a recording medium but also storing information in a memory unit.

<<Nucleic Acid Extracting Step>>

The nucleic acid extracting step is a step of extracting the nucleic acid having at least one of a full-length nucleotide sequence and a partial nucleotide sequence of rRNA or rDNA from cells in the well.

Extracting means destroying, for example, cellular membranes and cell walls to pick out the nucleic acid having at least one of a full-length nucleotide sequence and a partial nucleotide sequence of rRNA or rDNA.

As the method for extracting the nucleic acid having at least one of a full-length nucleotide sequence and a partial nucleotide sequence of rRNA or rDNA from cells, there is known a method of thermally treating cells at from 90 degrees C. through 100 degrees C. By a thermal treatment at 90 degrees C. or lower, there is a possibility that the nucleic acid may not be extracted. By a thermal treatment at 100 degrees C. or higher, there is a possibility that the nucleic acid may be decomposed. Here, it is applicable to perform thermal treatment with addition of a surfactant.

The surfactant is not particularly limited and may be appropriately selected depending on the intended purpose. Examples of the surfactant include ionic surfactants and nonionic surfactants. One of these surfactants may be used alone or two or more of these surfactants may be used in combination. Among these surfactants, nonionic surfactants are preferable because proteins are neither modified nor deactivated by nonionic surfactants, although depending on the addition amount of the nonionic surfactants.

Examples of the ionic surfactants include fatty acid sodium, fatty acid potassium, alpha-sulfo fatty acid ester sodium, sodium straight-chain alkyl benzene sulfonate, alkyl sulfuric acid ester sodium, alkyl ether sulfuric acid ester sodium, and sodium alpha-olefin sulfonate. One of these ionic surfactants may be used alone or two or more of these ionic surfactants may be used in combination. Among these ionic surfactants, fatty acid sodium is preferable and sodium dodecyl sulfate (SDS) is more preferable.

Examples of the nonionic surfactants include alkyl glycoside, alkyl polyoxyethylene ether (e.g., BRIJ series), octyl phenol ethoxylate (e.g., TRITON X series, IGEPAL CA series, NONIDET P series, and NIKKOL OP series), polysorbates (e.g., TWEEN series such as TWEEN 20), sorbitan fatty acid esters, polyoxyethylene fatty acid esters, alkyl maltoside, sucrose fatty acid esters, glycoside fatty acid esters, glycerin fatty acid esters, propylene glycol fatty acid esters, and fatty acid monoglyceride. One of these nonionic surfactants may be used alone or two or more of these nonionic surfactants may be used in combination. Among these nonionic surfactants, polysorbates are preferable.

The content of the surfactant is preferably 0.01% by mass or greater but 5.00% by mass or less relative to the total amount of the cell suspension in the well. When the content of the surfactant is 0.01% by mass or greater, the surfactant can be effective for extraction of nucleic acids. When the content of the surfactant is 5.00% by mass or less, inhibition against amplification can be prevented during PCR. As a numerical range in which both of these effects can be obtained, the range of 0.01% by mass or greater but 5.00% by mass or less is preferable.

The method described above may not be able to sufficiently extract a nucleic acid from a cell that has a cell wall. Examples of methods for such a case include an osmotic shock procedure, a freeze-thaw method, an enzymic digestive method, use of a DNA extraction kit, an ultrasonic treatment method, a French press method, and a homogenizer method. Among these methods, an enzymic digestive method is preferable because the method can save loss of extracted nucleic acids.

<<Other Steps>>

The other steps are not particularly limited and may be appropriately selected depending on the intended purpose. Examples of the other steps include an enzyme deactivating step.

—Enzyme Deactivating Step

The enzyme deactivating step is a step of deactivating an enzyme.

Examples of the enzyme include DNase, RNase, and an enzyme used in the nucleic acid extracting step in order to extract the nucleic acid having at least one of a full-length nucleotide sequence and a partial nucleotide sequence of rRNA or rDNA.

The method for deactivating an enzyme is not particularly limited and may be appropriately selected depending on the intended purpose. A known method can be suitably used.

Next, a nucleic acid testing method, a nucleic acid testing device, and a nucleic acid testing program using the device of the present disclosure will be described in detail below.

(Nucleic Acid Testing Method, Nucleic Acid Testing Device, and Nucleic Acid Testing Program)

The nucleic acid testing method of the present disclosure includes a step of using the device of the present disclosure and subjecting to amplification reaction, a testing target sample and a nucleic acid, which is provided in a defined copy number and has at least one of a full-length nucleotide sequence and a partial nucleotide sequence of rRNA or rDNA, to detect rRNA or rDNA contained in the testing target sample, and further includes other steps as needed.

Further, the nucleic acid testing method of the present disclosure is a nucleic acid testing method of subjecting a testing target sample and a nucleic acid, which is provided in a defined copy number and has at least one of a full-length nucleotide sequence and a partial nucleotide sequence of rRNA or rDNA, to amplification reaction to detect rRNA or rDNA contained in the testing target sample. The nucleic acid testing method includes a determining step of determining that a nucleic acid having at least one of a full-length nucleotide sequence and a partial nucleotide sequence of rRNA or rDNA is present in the testing target sample and a detection result is positive when the nucleic acid provided in the defined copy number and having at least one of a full-length nucleotide sequence and a partial nucleotide sequence of rRNA or rDNA and the testing target sample are both amplified, and determining that a nucleic acid having at least one of a full-length nucleotide sequence and a partial nucleotide sequence of rRNA or rDNA is absent or less than or equal to a limit of detection in the testing target sample and a detection result is negative when the nucleic acid provided in the defined copy number and having at least one of a full-length nucleotide sequence and a partial nucleotide sequence of rRNA or rDNA is amplified and the testing target sample is not amplified.

The nucleic acid testing method preferably includes an obtaining step of obtaining a result of amplification of the nucleic acid, which is provided in the defined copy number and has at least one of a full-length nucleotide sequence and a partial nucleotide sequence of rRNA or rDNA, and a result of amplification of the testing target sample, and an analyzing step of analyzing the result of amplification of the nucleic acid, which is provided in the defined copy number and has at least one of a full-length nucleotide sequence and a partial nucleotide sequence of rRNA or rDNA and the result of amplification of the testing target sample, and further includes other steps as needed.

A nucleic acid testing device of the present disclosure is a nucleic acid testing device used in detection of rRNA or rDNA contained in the testing target sample by subjecting a nucleic acid, which is provided in a defined copy number and has at least one of a full-length nucleotide sequence and a partial nucleotide sequence of rRNA or rDNA, and a testing target sample to amplification reaction. The nucleic acid testing device includes a determining unit configured to determine that a nucleic acid having at least one of a full-length nucleotide sequence and a partial nucleotide sequence of rRNA or rDNA is present in the testing target sample and a detection result is positive when the nucleic acid, which is provided in the defined copy number and has at least one of a full-length nucleotide sequence and a partial nucleotide sequence of rRNA or rDNA and the testing target sample are both amplified, and determine that a nucleic acid having at least one of a full-length nucleotide sequence and a partial nucleotide sequence of rRNA or rDNA is absent or less than or equal to a limit of detection in the testing target sample and a detection result is negative when the nucleic acid, which is provided in the defined copy number and has at least one of a full-length nucleotide sequence and a partial nucleotide sequence of rRNA or rDNA, is amplified and the testing target sample is not amplified. The nucleic acid testing device further includes other units as needed.

A nucleic acid testing program of the present disclosure is a nucleic acid testing program used in detection of rRNA or rDNA in the testing target sample by subjecting a nucleic acid, which is provided in a defined copy number and has at least one of a full-length nucleotide sequence and a partial nucleotide sequence of rRNA or rDNA and the testing target sample to amplification reaction.

The nucleic acid testing program causes a computer to execute a process including determining that a nucleic acid having at least one of a full-length nucleotide sequence and a partial nucleotide sequence of rRNA or rDNA is present in the testing target sample and a detection result is positive when the nucleic acid, which is provided in the defined copy number and has at least one of a full-length nucleotide sequence and a partial nucleotide sequence of rRNA or rDNA and the testing target sample are both amplified, and determining that a nucleic acid having at least one of a full-length nucleotide sequence and a partial nucleotide sequence of rRNA or rDNA is absent or less than or equal to a limit of detection in the testing target sample and a detection result is negative when the nucleic acid, which is provided in the defined copy number and has at least one of a full-length nucleotide sequence and a partial nucleotide sequence of rRNA or rDNA, is amplified and the testing target sample is not amplified. The nucleic acid testing program further causes a computer to execute any other process as needed.

Control being performed by, for example, a control unit of the nucleic acid testing device of the present disclosure has the same meaning as the nucleic acid testing method of the present disclosure being carried out. Therefore, details of the nucleic acid testing method of the present disclosure will also be specified through description of the nucleic acid testing device of the present disclosure. Further, the nucleic acid testing program of the present disclosure realizes the nucleic acid testing device of the present disclosure with the use of, for example, computers as hardware resources. Therefore, details of the nucleic acid testing program of the present disclosure will also be specified through description of the nucleic acid testing device of the present disclosure.

In the present disclosure, the nucleic acid testing method of the present disclosure, the nucleic acid testing device of the present disclosure, and the nucleic acid testing program of the present disclosure are based on the use of the device of the present disclosure having a nucleic acid dispensed in a defined copy number in each well with a coefficient of variation of a certain level or lower (with a filling accuracy of a certain level or higher), the nucleic acid having at least one of a full-length nucleotide sequence and a partial nucleotide sequence of rRNA or rDNA.

Use of the device of the present disclosure to subject a testing target sample to an amplification reaction makes it possible to detect a nucleic acid contained in the sample, and avoid a false-negative determination more infallibly, enable an accurate qualitative testing of whether positive or negative, and better improve negative determination accuracy particularly when the copy number of the nucleic acid in the sample is low.

According to the present disclosure, a negative determination result ensures that even if present in the testing target sample, a nucleic acid having at least one of a full-length nucleotide sequence and a partial nucleotide sequence of rRNA or rDNA is at least less than or equal to the defined copy number of the reference nucleic acid having at least one of a full-length nucleotide sequence and a partial nucleotide sequence of rRNA or rDNA, i.e., less than or equal to the limit of detection. That is, the present disclosure ensures, also from a quantitative point of view, an ambiguous “negative” determination result indicating absence of a nucleic acid having at least one of a full-length nucleotide sequence and a partial nucleotide sequence of rRNA or rDNA in the testing target sample.

In the present disclosure, “a low copy number” means that the copy number is low. The nucleic acid testing method of the present disclosure, the nucleic acid testing device of the present disclosure, and the nucleic acid testing program of the present disclosure are more effective for a testing target sample containing a nucleic acid in a low copy number. For example, the copy number of a nucleic acid having at least one of a full-length nucleotide sequence and a partial nucleotide sequence of rRNA or rDNA contained in a testing target sample is in the following order of preference (from lowest to highest): 1,000 or less, 500 or less, 200 or less, 100 or less, and 10 or less.

The copy number of the reference nucleic acid having at least one of a full-length nucleotide sequence and a partial nucleotide sequence of rRNA or rDNA, i.e., the nucleic acid provided in the defined copy number and having at least one of a full-length nucleotide sequence and a partial nucleotide sequence of rRNA or rDNA is a specific number and is a known copy number. The defined copy number is the same as in the device of the present disclosure. Hence, description about the defined copy number will be skipped.

The copy number of the nucleic acid provided in the defined copy number and having at least one of a full-length nucleotide sequence and a partial nucleotide sequence of rRNA or rDNA is in the following order of preference (from lowest to highest):1,000 or less, 500 or less, 200 or less, 100 or less, and 10 or less.

<Determining Step and Determining Unit>

The determining step is a step of using a nucleic acid, which is provided in a defined copy number and has at least one of a full-length nucleotide sequence and a partial nucleotide sequence of rRNA or rDNA, determining that a nucleic acid having at least one of a full-length nucleotide sequence and a partial nucleotide sequence of rRNA or rDNA is present in the testing target sample and a detection result is positive when the nucleic acid, which is provided in the defined copy number and has at least one of a full-length nucleotide sequence and a partial nucleotide sequence of rRNA or rDNA as positive control is amplified and the testing target sample is amplified, and determining that a nucleic acid having at least one of a full-length nucleotide sequence and a partial nucleotide sequence of rRNA or rDNA is absent or less than a limit of detection in the testing target sample and a detection result is negative when the nucleic acid, which is provided in the defined copy number and has at least one of a full-length nucleotide sequence and a partial nucleotide sequence of rRNA or rDNA as positive control is amplified and the testing target sample is not amplified. The determining step is performed by the determining unit.

Because the reference nucleic acid to be used as a control in quantitative PCR is prepared by a serial dilution method as in the existing techniques, there is a possibility that the result of the quantitative PCR measurement will have a large variation (e.g., CT (Threshold cycle) value variation) when the copy number of the nucleic acid is low, and a highly accurate determination of the detection result may be impossible.

As compared, the nucleic acid testing method of the present disclosure can suppress variation of the result of quantitative PCR measurement (e.g., CT value variation) even when the copy number of the nucleic acid is low and can perform a highly accurate determination of the detection result, based on use of the device of the present disclosure having a nucleic acid located in the defined copy number in the wells at a high accuracy, the nucleic acid having at least one of a full-length nucleotide sequence and a partial nucleotide sequence of rRNA or rDNA.

Accordingly, the nucleic acid testing method of the present disclosure can suppress variation of the result of quantitative PCR measurement (e.g., CT value variation) of a nucleic acid in a low copy number, and can ensure a high reliability for the result of detection of the reference nucleic acid. Therefore, even if the copy number of the nucleic acid contained in the testing target sample is low, the nucleic acid testing method can avoid false-negative determination of the detection result more infallibly, better improve negative determination accuracy, and enable an accurate qualification of whether positive or negative.

Furthermore, according to the nucleic acid testing method of the present disclosure, it is possible to locate a nucleic acid in the wells in different defined copy numbers highly accurately even if the copy numbers are low. Therefore, the nucleic acid testing method can accurately quantify the amount of the nucleic acid contained in the testing target sample, even if the copy number of the nucleic acid contained in the testing target sample is low.

For example, when the copy number of the reference nucleic acid having at least one of a full-length nucleotide sequence and a partial nucleotide sequence of rRNA or rDNA used as a control is not specified as in the existing techniques, for example, determination about detection of nucleic acid such as rRNA or rDNA made based on the result of amplification of the testing target sample (a sample that may possibly contain rRNA or rDNA) and the result of amplification of the reference nucleic acid having at least one of a full-length nucleotide sequence and a partial nucleotide sequence of rRNA or rDNA will result as presented in Table 2 below.

TABLE 2

| | | Reference nucleic acid having at least one of full-length nucleotide | |
|---|---|---|---|
| | | sequence and partial nucleotide sequence of rRNA or rDNA (copy | |
| | | number of reference nucleic acid not defined) | |
| | | + | − |
| Testing target sample | + | (1) Positive (highly | (3) Reconsideration of PCR reaction |
| (sample possibly | | probable) | system and reconsideration of copy |
| containing rRNA or | | | number of reference nucleic acid |
| rDNA) | | | having at least one of full-length |
| | | | nucleotide sequence and partial |
| | | | nucleotide sequence of rRNA or rDNA |
| | | | are needed |
| | − | (2) Negative or false- | (4) Reconsideration of PCR reaction |
| | | negative (impossible to | system and reconsideration of copy |
| | | detect whether negative | number of reference nucleic acid |
| | | or false-negative) | having at least one of full-length |
| | | | nucleotide sequence and partial |
| | | | nucleotide sequence of rRNA or rDNA |
| | | | are needed |

As presented in Table 2, amplification reaction results include four patterns, namely (1) a case where amplification is observed in both of the testing target sample (the sample that may possibly contain rRNA or rDNA) and the reference nucleic acid having at least one of a full-length nucleotide sequence and a partial nucleotide sequence of rRNA or rDNA, (2) a case where amplification is observed in the reference nucleic acid having at least one of a full-length nucleotide sequence and a partial nucleotide sequence of rRNA or rDNA, but amplification is not observed in the testing target sample (the sample that may possibly contain rRNA or rDNA), (3) a case where amplification is observed in the testing target sample (the sample that may possibly contain rRNA or rDNA), but amplification is not observed in the reference nucleic acid having at least one of a full-length nucleotide sequence and a partial nucleotide sequence of rRNA or rDNA, and (4) a case where amplification is observed in neither the testing target sample (the sample that may possibly contain rRNA or rDNA) nor the reference nucleic acid having at least one of a full-length nucleotide sequence and a partial nucleotide sequence of rRNA or rDNA.

When the copy number of the reference nucleic acid having at least one of a full-length nucleotide sequence and a partial nucleotide sequence of rRNA or rDNA is not specified as in Table 2, the results (1) to (4) described above can be determined as follows.

In the case of (1), it is possible to confirm that the experiment by PCR reaction has been successful. Further, it is possible to confirm that a testing target nucleic acid (nucleic acid having at least one of a full-length nucleotide sequence and a partial nucleotide sequence of rRNA or rDNA) is present in the testing target sample.

In the case of (2), it is possible to confirm that the experiment by PCR reaction has been successful. However, detection of whether the testing target nucleic acid (nucleic acid having at least one of a full-length nucleotide sequence and a partial nucleotide sequence of rRNA or rDNA) is present in the testing target sample has been unsuccessful. Because the copy number of the reference nucleic acid having at least one of a full-length nucleotide sequence and a partial nucleotide sequence of rRNA or rDNA is not defined, it is impossible to specify which of the following cases is pertinent, namely a case where the testing target nucleic acid (nucleic acid having at least one of a full-length nucleotide sequence and a partial nucleotide sequence of rRNA or rDNA) is absent in the testing target sample (negative) and a case where the testing target nucleic acid is present in the testing target sample, but could not be identified and was erroneously determined as negative (false-negative). Particularly, when the copy number of the nucleic acid is a low copy number, the determination of whether negative or false-negative is more difficult.

In the case of (3) and (4), because amplification is not observed in the reference nucleic acid having at least one of a full-length nucleotide sequence and a partial nucleotide sequence of rRNA or rDNA, for example, it is considered that the PCR has not progressed due to some causes (for example, reaction temperature conditions, preparation of the reference nucleic acid having at least one of a full-length nucleotide sequence and a partial nucleotide sequence of rRNA or rDNA, a thermal cycler, and settings of the real-time PCR device), or that the copy number of the reference nucleic acid having at least one of a full-length nucleotide sequence and a partial nucleotide sequence of rRNA or rDNA is insufficient with respect to the limit of detection, and it is determined that “reconsideration of the PCR system and reconsideration of the copy number of the reference nucleic acid having at least one of a full-length nucleotide sequence and a partial nucleotide sequence of rRNA or rDNA are needed”. When the copy number of the reference nucleic acid having at least one of a full-length nucleotide sequence and a partial nucleotide sequence of rRNA or rDNA is not specified, the copy number has a large variation, and the probability that the copy number is higher than or equal to the limit of detection is low. This inevitably increases the frequency that the test results of (3) and (4) will be obtained. Therefore, when the copy number of the reference nucleic acid having at least one of a full-length nucleotide sequence and a partial nucleotide sequence of rRNA or rDNA is not defined, there is a need for performing a test using a copy number that is twice or three times as high as the limit of detection.

On the other hand, when the copy number of the reference nucleic acid having at least one of a full-length nucleotide sequence and a partial nucleotide sequence of rRNA or rDNA is defined as in the present disclosure, for example, determination about detection of the testing target sample (a sample that may possibly contain rRNA or rDNA) made based on the result of amplification of the testing target sample (the sample that may possibly contain rRNA or rDNA) and the result of amplification of the reference nucleic acid which is provided in the defined copy number and having at least one of a full-length nucleotide sequence and a partial nucleotide sequence of rRNA or rDNA will be assigned according to Table 3 below.

TABLE 3

| | | Reference nucleic acid having at least one of full-length nucleotide | |
|---|---|---|---|
| | | sequence and partial nucleotide sequence of rRNA or rDNA (in | |
| | | defined copy number) | |
| | | + | − |
| Testing target sample | + | (1) Positive (ascertained) | (3) Reconsideration of PCR reaction |
| (sample possibly | | | system and reconsideration of copy |
| containing rRNA or | | | number of reference nucleic acid |
| rDNA) | | | having at least one of full-length |
| | | | nucleotide sequence and partial |
| | | | nucleotide sequence of rRNA or |
| | | | rDNA are needed |
| | − | (2) Negative (ascertained) | (4) Reconsideration of PCR reaction |
| | | (copy number of testing | system and reconsideration of copy |
| | | target is less than or equal | number of reference nucleic acid |
| | | to limit of detection | having at least one of full-length |
| | | | nucleotide sequence and partial |
| | | | nucleotide sequence of rRNA or |
| | | | rDNA are needed |

When the defined copy number of the reference nucleic acid having at least one of a full-length nucleotide sequence and a partial nucleotide sequence of rRNA or rDNA is assigned according to Table 3, the results (1) to (4) described above can be determined as follows.

In the case of (1), it is possible to ascertain that the experiment by PCR reaction has been successful. Further, it is possible to ascertain that the testing target nucleic acid (nucleic acid having at least one of a full-length nucleotide sequence and a partial nucleotide sequence of rRNA or rDNA) is present in the testing target sample. Even when the copy number of the nucleic acid is a low copy number, the “positive” determination result can be ensured.

In the case of (2), it is possible to say that the testing target nucleic acid (nucleic acid having at least one of a full-length nucleotide sequence and a partial nucleotide sequence of rRNA or rDNA) is absent in the testing target sample because the testing target nucleic acid is less than or equal to the limit of detection and has not been detected. In the case of (2), it is impossible to specify whether negative or false-negative according to Table 2, whereas it is possible to conclude that the result is “negative” according to Table 3 of the present disclosure because the copy number of the reference nucleic acid having at least one of a full-length nucleotide sequence and a partial nucleotide sequence of rRNA or rDNA is specified.

The present disclosure makes it possible to more securely exclude false-negative determination. The present disclosure can reduce false-negative and ensure a “negative” determination result based on the reasoning that the testing target nucleic acid is at least less than or equal to the defined copy number of the reference nucleic acid having at least one of a full-length nucleotide sequence and a partial nucleotide sequence of rRNA or rDNA, or less than or equal to the limit of detection.

In the case of (3) and (4), because amplification is not observed in the reference nucleic acid provided in the defined copy number and having at least one of a full-length nucleotide sequence and a partial nucleotide sequence of rRNA or rDNA, for example, it is estimated that the PCR reaction has not progressed due to some causes (for example, reaction temperature conditions, preparation of the reference nucleic acid having at least one of a full-length nucleotide sequence and a partial nucleotide sequence of rRNA or rDNA, a thermal cycler, and settings of the real-time PCR device), or that the copy number of the reference nucleic acid having at least one of a full-length nucleotide sequence and a partial nucleotide sequence of rRNA or rDNA is insufficient with respect to the limit of detection, and it is determined that “reconsideration of the PCR reaction system and reconsideration of the defined copy number of the reference nucleic acid having at least one of a full-length nucleotide sequence and a partial nucleotide sequence of rRNA or rDNA are needed”.

In the nucleic acid testing method of the present disclosure, it is applicable that the limit of detection of the testing target sample (the sample that may possibly contain rRNA or rDNA) be comparable to the limit of detection of the nucleic acid provided in the defined copy number and having at least one of a full-length nucleotide sequence and a partial nucleotide sequence of rRNA or rDNA.

This makes it possible to determine a limit of detection, which is obtained based on a result of amplification of the nucleic acid provided in the defined copy number and having at least one of a full-length nucleotide sequence and a partial nucleotide sequence of rRNA or rDNA, as a limit of detection of the testing target nucleic acid.

The nucleic acid testing method of the present disclosure may fill a well in which the testing target sample (a sample that may possibly contain rRNA or rDNA) is located, except a well in which the nucleic acid having at least one of a full-length nucleotide sequence and a partial nucleotide sequence of rRNA or rDNA is contained in the defined copy number, with an amplifiable reagent different from the testing target sample (the sample that may possibly contain rRNA or rDNA), and subject the nucleic acid provided in the defined copy number and having at least one of a full-length nucleotide sequence and a partial nucleotide sequence of rRNA or rDNA, the testing target sample (the sample that may possibly contain rRNA or rDNA), and the amplifiable reagent to amplification reaction.

That is, the amplifiable reagent different from the testing target sample (the sample that may possibly contain rRNA or rDNA) is used in a certain amount and located in the same well in which the testing target sample (the sample that may possibly contain rRNA or rDNA) is located, and is subjected to amplification reaction. If the amplifiable reagent is amplified, it is possible to confirm that the amplification reaction is successful in the well in which the amplifiable reagent is located. This better ensures the reliability of the result of amplification of the testing target sample (the sample that may possibly contain rRNA or rDNA) in the same well in which the amplifiable reagent is located. Here, the certain amount needs at least to be a sufficiently detectable amount.

Hence, according to the nucleic acid testing method, it is possible to determine the results more infallibly by determining that the testing target nucleic acid (nucleic acid having at least one of a full-length nucleotide sequence and a partial nucleotide sequence of rRNA or rDNA) is present and a detection result is positive when all of the nucleic acid provided in the defined copy number and having at least one of a full-length nucleotide sequence and a partial nucleotide sequence of rRNA or rDNA, the amplifiable reagent, and the testing target sample (the sample that may possibly contain rRNA or rDNA) are amplified, and determining that the testing target nucleic acid (nucleic acid having at least one of a full-length nucleotide sequence and a partial nucleotide sequence of rRNA or rDNA) is absent or less than or equal to the limit of detection and a detection result is negative when the nucleic acid provided in the defined copy number and having at least one of a full-length nucleotide sequence and a partial nucleotide sequence of rRNA or rDNA and the amplifiable reagent are amplified but the testing target sample (the sample that may possibly contain rRNA or rDNA) is not amplified.

The amplifiable reagent is not particularly limited and may be appropriately selected depending on the intended purpose as long as the amplifiable reagent is a nucleic acid different from the testing garget nucleic acid (nucleic acid having at least one of a full-length nucleotide sequence and a partial nucleotide sequence of rRNA or rDNA). For example, the nucleic acid described in the foregoing section “-Nucleic acid-” may be used. In the nucleic acid testing method of the present disclosure, a naturally non-existent non-natural nucleic acid may be used as the amplifiable reagent, because a naturally non-existent non-natural nucleic acid can be clearly distinguished from the testing target nucleic acid (nucleic acid having at least one of a full-length nucleotide sequence and a partial nucleotide sequence of rRNA or rDNA).

Further, in the nucleic acid testing method of the present disclosure, it is applicable that the well in which the nucleic acid having at least one of a full-length nucleotide sequence and a partial nucleotide sequence of rRNA or rDNA is contained in the defined copy number include one well in which the nucleic acid having at least one of a full-length nucleotide sequence and a partial nucleotide sequence of rRNA or rDNA is contained in a predetermined defined copy number and another one well in which the nucleic acid having at least one of a full-length nucleotide sequence and a partial nucleotide sequence of rRNA or rDNA is contained in a defined copy number different from the defined copy number in the one well, and that the nucleic acid testing method include: subjecting the nucleic acids having at least one of a full-length nucleotide sequence and a partial nucleotide sequence of rRNA or rDNA contained in the one well and the another one well varied in defined copy number, and the testing target sample to amplification reaction; and comparing results of amplification of the nucleic acids having at least one of a full-length nucleotide sequence and a partial nucleotide sequence of rRNA or rDNA contained in the respective defined copy numbers with a result of amplification of the testing target sample to determine an amount of rRNA or rDNA contained in the testing target sample.

Furthermore, with the use of the device of the present disclosure including one well and another well in which a nucleic acid having at least one of a full-length nucleotide sequence and a partial nucleotide sequence of rRNA or rDNA are contained in different defined copy numbers, the nucleic acid testing method of the present disclosure can quantify the amount of the testing target nucleic acid (nucleic acid having at least one of a full-length nucleotide sequence and a partial nucleotide sequence of rRNA or rDNA) contained in the testing target sample.

That is, using the device, the nucleic acid testing method can compare results of amplification of the nucleic acids provided in different defined copy numbers and having at least one of a full-length nucleotide sequence and a partial nucleotide sequence of rRNA or rDNA with a result of amplification of the testing target sample (the sample that may possibly contain rRNA or rDNA), and determine the amount of the testing target nucleic acid (nucleic acid having at least one of a full-length nucleotide sequence and a partial nucleotide sequence of rRNA or rDNA).

Examples of the method for comparing results of amplification of the nucleic acids provided in different defined copy numbers and having at least one of a full-length nucleotide sequence and a partial nucleotide sequence of rRNA or rDNA with a result of amplification of the testing target sample (the sample that may possibly contain rRNA or rDNA), and determining the amount of the testing target nucleic acid (nucleic acid having at least one of a full-length nucleotide sequence and a partial nucleotide sequence of rRNA or rDNA) include a method of generating a calibration curve based on the results of amplification of the nucleic acids provided in different defined copy numbers and having at least one of a full-length nucleotide sequence and a partial nucleotide sequence of rRNA or rDNA and quantifying the amount of the testing target nucleic acid based on the result of amplification of the testing target sample (the sample that may possibly contain rRNA or rDNA) and the calibration curve.

<Testing Result Obtaining Step and Testing Result Obtaining Unit>

The testing result obtaining step is a step of obtaining a result of amplification of the nucleic acid provided in the defined copy number and having at least one of a full-length nucleotide sequence and a partial nucleotide sequence of rRNA or rDNA and a result of amplification of the testing target sample (the sample that may possibly contain rRNA or rDNA), and is performed by a testing result obtaining unit. The testing result obtaining unit 131 is configured to obtain a result of amplification of the nucleic acid provided in the defined copy number and having at least one of a full-length nucleotide sequence and a partial nucleotide sequence of rRNA or rDNA and a result of amplification of the testing target sample (the sample that may possibly contain rRNA or rDNA) obtained from PCR reactions. The data of the obtained results of amplification is stored in a testing result database 141.

<Testing Result Analyzing Step and Testing Result Analyzing Unit>

The testing result analyzing step is a step of analyzing the obtained result of amplification of the nucleic acid provided in the defined copy number and having at least one of a full-length nucleotide sequence and a partial nucleotide sequence of rRNA or rDNA and the obtained result of amplification of the testing target sample (the sample that may possibly contain rRNA or rDNA), and is performed by a testing result analyzing unit.

The testing result analyzing unit 132 is configured to obtain the data of the results of amplification stored in the testing result database 141, and based on the data, analyze whether amplification is observed in the nucleic acid provided in the defined copy number and having at least one of a full-length nucleotide sequence and a partial nucleotide sequence of rRNA or rDNA and whether amplification is observed in the testing target sample (the sample that may possibly contain rRNA or rDNA).

The procedure of a nucleic acid testing program of the present disclosure can be executed using a computer including a control unit constituting a nucleic acid testing device.

The hardware configuration and the functional configuration of the nucleic acid testing device will be described below.

<Hardware Configuration of Nucleic Acid Testing Device>

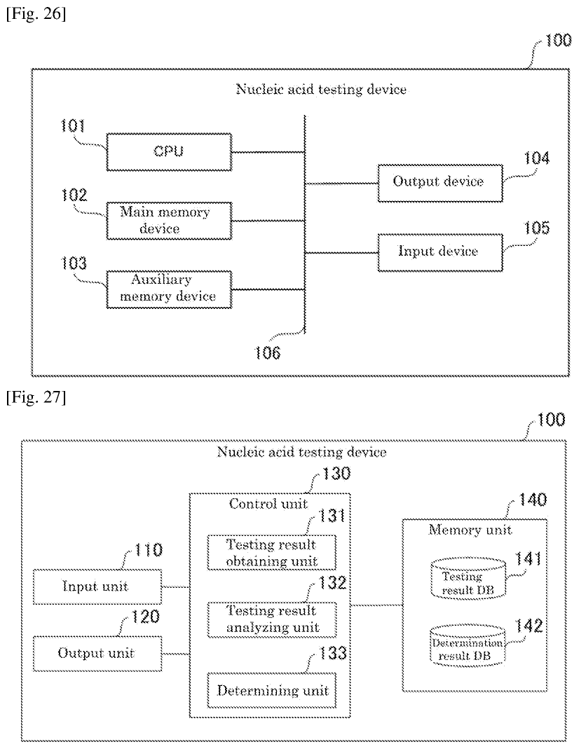

FIG. 26 is a block diagram illustrating an example of the hardware configuration of a nucleic acid testing device 100.

As illustrated in FIG. 26, the nucleic acid testing device 100 includes units such as a CPU (Central Processing Unit) 101, a main memory device 102, an auxiliary memory device 103, an output device 104, and an input device 105. These units are coupled to one another through a bus 106.

The CPU 101 is a processing device configured to execute various controls and operations. The CPU 101 realizes various functions by executing OS (Operating System) and programs stored in, for example, the main memory device 102. That is, in the present example, the CPU 101 functions as a control unit 130 of the nucleic acid testing device 100 by executing the nucleic acid testing program.

The CPU 101 also controls the operation of the entire nucleic acid testing device 100. In the present example, the CPU 101 is used as the device configured to control the operation of the entire nucleic acid testing device 100. However, this is non-limiting. For example, FPGA (Field Programmable Gate Array) may be used.

The nucleic acid testing program and various databases need not indispensably be stored in, for example, the main memory device 102 and the auxiliary memory device 103. The nucleic acid testing program and various databases may be stored in, for example, another information processing device that is coupled to the nucleic acid testing device 100 through, for example, the Internet, a LAN (Local Area Network), and a WAN (Wide Area Network). The nucleic acid testing device 100 may receive the nucleic acid testing program and various databases from such another information processing device and execute the program and databases.

The main memory device 102 is configured to store various programs and store, for example, data needed for execution of the various programs.

The main memory device 102 includes a ROM (Read Only Memory) and a RAM (Random Access Memory) that are not illustrated.

The ROM is configured to store various programs such as BIOS (Basic Input/Output System).

The RAM functions as a work area to be developed when the various programs stored in the ROM are executed by the CPU 101. The RAM is not particularly limited and may be appropriately selected depending on the intended purpose. Examples of the RAM include a DRAM (Dynamic Random Access Memory) and a SRAM (Static Random Access Memory).

The auxiliary memory device 103 is not particularly limited and may be appropriately selected depending on the intended purpose as long as the auxiliary memory device 103 can store various information. Examples of the auxiliary memory device 103 include portable memory devices such as a CD (Compact Disc) drive, a DVD (Digital Versatile Disc) drive, and a BD (Blue-ray (registered trademark) Disc) drive.

For example, a display or a speaker can be used as the output device 104. The display is not particularly limited and a known display can be appropriately used. Examples of the display include a liquid crystal display and an organic EL display.

The input device 105 is not particularly limited and a known input device can be appropriately used as long as the input device can receive various requests to the nucleic acid testing device 100. Examples of the input device include a keyboard, a mouse, and a touch panel.

The hardware configuration as described above can realize the process functions of the nucleic acid testing device 100.

<Functional Configuration of Nucleic Acid Testing Device>

FIG. 27 is a diagram illustrating an example of the functional configuration of the nucleic acid testing device 100.

As illustrated in FIG. 27, the nucleic acid testing device 100 includes an input unit 110, an output unit 120, the control unit 130, and a memory unit 140.

The control unit 130 includes the testing result obtaining unit 131, the testing result analyzing unit 132, and a determining unit 133. The control unit 130 is configured to control the entire nucleic acid testing device 100.

The memory unit 140 includes the testing result database 141 and a determination result database 142. Hereinafter, “database” may be referred to as “DB”.

The testing result obtaining unit 131 is configured to obtain a result of amplification of the nucleic acid provided in the defined copy number and having at least one of a full-length nucleotide sequence and a partial nucleotide sequence of rRNA or rDNA and a result of amplification of the testing target sample (the sample that may possibly contain rRNA or rDNA) obtained from PCR reactions. The control unit 130 is configured to store data of the obtained results of amplification in the testing result DB 141.

The testing result analyzing unit 132 is configured to analyze the result of amplification of the nucleic acid provided in the defined copy number and having at least one of a full-length nucleotide sequence and a partial nucleotide sequence of rRNA or rDNA and the result of amplification of the testing target sample (the sample that may possibly contain rRNA or rDNA), using the data of the results of amplification stored in the testing result DB 141 of the memory unit 140.

The determining unit 133 is configured to determine “positive” and “negative” when the classifications described below are applicable, based on the results of the analyses of the testing result analyzing unit 132.

(1) When the nucleic acid provided in the defined copy number and having at least one of a full-length nucleotide sequence and a partial nucleotide sequence of rRNA or rDNA is amplified and the testing target sample (the sample that may possibly contain rRNA or rDNA) is amplified, it is determined that the testing target nucleic acid (nucleic acid having at least one of a full-length nucleotide sequence and a partial nucleotide sequence of rRNA or rDNA) is present and the testing result is positive.

(2) When the nucleic acid provided in the defined copy number and having at least one of a full-length nucleotide sequence and a partial nucleotide sequence of rRNA or rDNA is amplified and the testing target sample (the sample that may possibly contain rRNA or rDNA) is not amplified, it is determined that the testing target nucleic acid (nucleic acid having at least one of a full-length nucleotide sequence and a partial nucleotide sequence of rRNA or rDNA) is absent or less than or equal to the limit of detection and the testing result is negative.

In addition to the determinations of (1) and (2) above, the determining unit 133 may make a determination of, for example, failure of experiment when the cases of (3) and (4) in Table 3 are applicable.

The control unit 130 is configured to store the determination result of the determining unit 133 in the determination result DB 142.

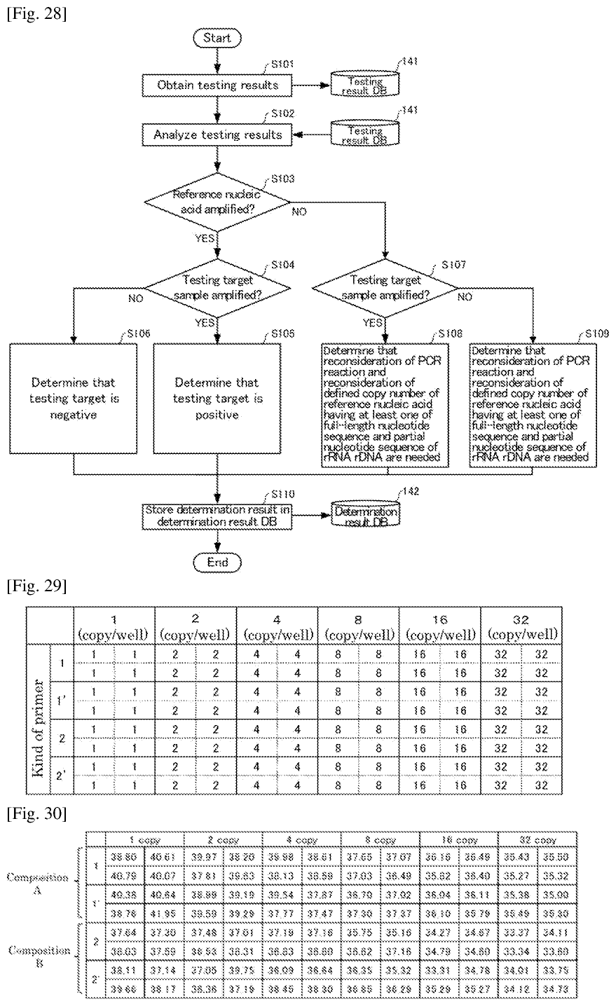

Next, the process procedure of the nucleic acid testing program of the present disclosure will be described. FIG. 28 is a flowchart illustrating an example of the process procedure of the nucleic acid testing program by the control unit 130 of the nucleic acid testing device 100.

In the steps S101, the testing result obtaining unit 131 of the control unit 130 of the nucleic acid testing device 100 obtains a result of amplification of a nucleic acid provided in the defined copy number and having at least one of a full-length nucleotide sequence and a partial nucleotide sequence of rRNA or rDNA and a result of amplification of a testing target sample (a sample that may possibly contain rRNA or rDNA) obtained from PCR reactions, and moves the flow to the step S102. In the step S101, the control unit 130 stores the data of the results of amplification obtained by the testing result obtaining unit 131 in the testing result DB 141 of the memory unit 140.

In the step S102, the testing result analyzing unit 132 of the control unit 130 of the nucleic acid testing device 100 obtains the data of the results of amplification stored in the testing result DB 141. Then, the testing result analyzing unit 132 analyzes the respective results as to whether amplification is observed in the nucleic acid provided in the defined copy number and having at least one of a full-length nucleotide sequence and a partial nucleotide sequence of rRNA or rDNA and whether amplification is observed in the testing target nucleic acid, and moves the flow to the step S103.

In the step S103, the determining unit 133 of the control unit 130 of the nucleic acid testing device 100 moves the flow to the step S104 when amplification is observed in the nucleic acid provided in the defined copy number and having at least one of a full-length nucleotide sequence and a partial nucleotide sequence of rRNA or rDNA, based on the result of the analysis by the testing result analyzing unit 132. On the other hand, the determining unit 133 moves the flow to the step S107 when amplification is not observed in the nucleic acid provided in the defined copy number and having at least one of a full-length nucleotide sequence and a partial nucleotide sequence of rRNA or rDNA.

In the step S104, the determining unit 133 moves the flow to the step S105 when amplification is observed in the testing target sample (the sample that may possibly contain rRNA or rDNA), based on the result of the analysis by the testing result analyzing unit 132. On the other hand, the determining unit 133 moves the flow to step S106 when amplification is not observed in the testing target sample (the sample that may possibly contain rRNA or rDNA).

In the step S105, the determining unit 133 determines that the testing target nucleic acid (nucleic acid having at least one of a full-length nucleotide sequence and a partial nucleotide sequence of rRNA or rDNA) is present and the testing result is positive, based on the results indicating that the nucleic acid provided in the defined copy number and having at least one of a full-length nucleotide sequence and a partial nucleotide sequence of rRNA or rDNA is amplified and that the testing target sample (the sample that may possibly contain rRNA or rDNA) is amplified, and moves the flow to the step S110.

In the step S106, the determining unit 133 determines that the testing target nucleic acid (nucleic acid having at least one of a full-length nucleotide sequence and a partial nucleotide sequence of rRNA or rDNA) is absent or less than or equal to the limit of detection and the testing result is negative, based on the results indicating that the nucleic acid provided in the defined copy number and having at least one of a full-length nucleotide sequence and a partial nucleotide sequence of rRNA or rDNA is amplified and that the testing target sample (the sample that may possibly contain rRNA or rDNA) is not amplified, and moves the flow to the step S110.

In the step S107, the determining unit 133 moves the flow to the step S108 when amplification is observed in the testing target sample (the sample that may possibly contain rRNA or rDNA), based on the result of the analysis by the testing result analyzing unit 132. On the other hand, the determining unit 133 moves the flow to step S109 when amplification is not observed in the testing target sample (the sample that may possibly contain rRNA or rDNA).

In the step S108, the determining unit 133 determines that reconsideration of the PCR reaction system and reconsideration of the defined copy number of the reference nucleic acid having at least one of a full-length nucleotide sequence and a partial nucleotide sequence of rRNA or rDNA are needed, based on the results indicating that the nucleic acid provided in the defined copy number and having at least one of a full-length nucleotide sequence and a partial nucleotide sequence of rRNA or rDNA is not amplified and that the testing target sample (the sample that may possibly contain rRNA or rDNA) is amplified, and moves the flow to the step S110.

In the step S109, the determining unit 133 determines that reconsideration of the PCR reaction system and reconsideration of the defined copy number of the reference nucleic acid having at least one of a full-length nucleotide sequence and a partial nucleotide sequence of rRNA or rDNA are needed, based on the results indicating that the nucleic acid provided in the defined copy number and having at least one of a full-length nucleotide sequence and a partial nucleotide sequence of rRNA or rDNA is not amplified and that the testing target sample (the sample that may possibly contain rRNA or rDNA) is not amplified, and moves the flow to the step S110.

In the step S110, the control unit 130 stores the determination result made by the determining unit 133 in the determination result DB 142 of the memory unit 140 and terminates the flow.

In the present disclosure, it is at least needed to perform the determination in the step S105 or the step S106, and a mode in which the flow is terminated without moving to the step S107 is possible when amplification is not observed in the nucleic acid provided in the defined copy number and having at least one of a full-length nucleotide sequence and a partial nucleotide sequence of rRNA or rDNA.

(Gene Testing Method)

A gene testing method of the present disclosure is a gene testing method targeting rRNA or rDNA. The gene testing method manages accuracy of an accuracy managing target, using a standard substance, of which absolute number is prescribed by counting the rRNA or rDNA, where the absolute number contains uncertainty.

The accuracy managing target is not particularly limited and may be appropriately selected depending on the intended purpose. Examples of the accuracy managing target include a gene testing/analyzing device, a reagent, and a primer used in the gene testing method.

The gene testing method of the present disclosure is based on use of the device of the present disclosure. Use of the device of the present disclosure makes it possible to perform gene testing at a high sensitivity and at a high accuracy.

The standard substance means the same as the nucleic acid provided in the defined copy number and having at least one of a full-length nucleotide sequence and a partial nucleotide sequence of rRNA or rDNA used in the device of the present disclosure. Therefore, description about the standard substance will be skipped. Other terms also mean the same as in the device of the present disclosure. Therefore, description about the terms will be skipped.

### EXAMPLES

The present disclosure will be described below by way of Example. The present disclosure should not be construed as being limited to the Example.

### Example

<Production of Device>

A device was produced in the manner described below.

<Preparation of Nucleic Acid Sample>

—Gene Recombinant Yeast—

For producing a recombinant, a budding yeast YIL015W BY4741 (ATCC, ATCC4001408) was used as a carrier cell for one copy of a specific nucleotide sequence.

The specific nucleotide sequence was a pig 12S rRNA nucleotide sequence (FASMAC, see SEQ ID NO. 1). In the form of a plasmid produced by arranging the specific nucleotide sequence in tandem with URA3, which was a selectable marker, one copy of the specific nucleotide sequence was introduced into yeast genome DNA by homologous recombination, targeting a BAR1 region of the carrier cell, to produce a gene recombinant yeast. In the present Example, only a partial sequence of the pig 12S rRNA nucleotide sequence was used as the specific nucleotide sequence. However, it would also be possible to locate other specific nucleotide sequences in different wells and perform testing of a plurality of samples simultaneously with one plate (one device).

—Culturing and Cell-Cycle Control—

In an Erlenmeyer flask, a 90-mL fraction of the gene recombinant yeast cultured in 50 g/L of a YPD medium (Takara Bio Inc., CLN-630409) was mixed with 900 microliters of α1-MATING FACTOR ACETATE SALT (Sigma-Aldrich Co., LLC, T6901-5MG, hereinafter referred to as “a factor”) prepared to 500 micrograms/mL with a Dulbecco's phosphate buffered saline (Thermo Fisher Scientific Inc., 14190-144, hereinafter referred to as “DPBS”).

Next, the resultant was incubated with a bioshaker (Taitec Corporation, BR-23FH) at a shaking speed of 250 rpm at a temperature of 28 degrees C. for 2 hours, to synchronize the yeast at a G0/G1 phase, to obtain a yeast suspension.

—Fixing—

Forty-five milliliters of the synchronization-confirmed yeast suspension was transferred to a centrifuge tube (As One Corporation, VIO-50R) and centrifuged with a centrifugal separator (Hitachi, Ltd., F16RN,) at a rotation speed of 3,000 rpm for 5 minutes, with subsequent supernatant removal, to obtain yeast pellets.

Four milliliters of formalin (Wako Pure Chemical Industries, Ltd., 062-01661) was added to the obtained yeast pellets, and the resultant was left to stand still for 5 minutes, then centrifuged with subsequent supernatant removal, and suspended with addition of 10 mL of ethanol, to obtain a fixed yeast suspension.

—Nuclear Staining—

Two hundred microliters of the fixed yeast suspension was fractionated, washed with DPBS once, and resuspended in 480 microliters of DPBS.

Next, to the resultant, 20 microliters of 20 mg/mL RNase A (Nippon Gene Co., Ltd., 318-06391) was added, followed by incubation with a bioshaker at 37 degrees C. for 2 hours.

Next, to the resultant, 25 microliters of 20 mg/mL proteinase K (Takara Bio Inc., TKR-9034) was added, followed by incubation with PETIT COOL (Waken B Tech Co., Ltd., PETIT COOL MINI T-C) at 50 degrees C. for 2 hours.

Finally, to the resultant, 6 microliters of 5 mM SYTOX GREEN NUCLEIC ACID STAIN (Thermo Fisher Scientific Inc., 57020) was added, followed by staining in a light-shielded environment for 30 minutes.

—Dispersing

The stained yeast suspension was subjected to dispersion treatment using an ultrasonic homogenizer (Yamato Scientific Co., Ltd., LUH150,) at a power output of 30% for 10 seconds, to obtain a yeast suspension ink.

<Filling of Nucleic Acid Samples>

—Filling of Series of Low-Concentration Nucleic Acid Samples

—Dispensing of Yeast Suspension with Number Counting—

After a filling container (96-well flat bottom plate (Watson Co., Ltd., 4846-96-FS)) was filled with a dissolving liquid for dissolving cell walls in an amount of 4 microliters per well beforehand, the series of low-concentration nucleic acid samples were dispensed one cell per well, using a cell sorter (Sony Corporation, SH800Z).

Next, with a Tris-EDTA (TE) buffer (Thermo Fisher Scientific Inc., AM9861) serving as a cell wall dissolving liquid and ColE1 DNA (Nippon Gene Co., Ltd., 312-00434), ColE1/TE was prepared at 5 ng/microliter. With ColE1/TE, a Zymolyase solution of Zymolyase® 100T (Nacalai Tesque Inc., 07665-55) was prepared at 1 mg/mL.

Note that dispensing by a cell sorter was performed in a single cell mode, with an analysis of the cell cycle at an excitation wavelength of 488 nm, to select only a region in which G0/G1 phase cells were present.

—Extraction of Nucleic Acids from Dispensed Yeast Cells—

For extraction of nucleic acids from the yeast cells, the filling container was incubated at 37 degrees C. for 30 minutes, to dissolve the cell walls (extraction of nucleic acids), and then thermally treated at 95 degrees C. for 2 minutes.

<Test for Evaluating Performance of Primer Using Pig-Derived Standard Substance for Nucleic Acid Testing>

It is said that efficiency and sensitivity of qPCR reaction are greatly dependent on the performance of primers. If a target in a high copy number is used for evaluation of the performance, a sufficient performance difference cannot appear. Hence, by an amplification test performed at a low copy template concentration made available by the present disclosure, what degree of performance difference would appear between two different primer-and-probe sets was tested.

In order to formulate a high-sensitivity, high-accuracy scheme for testing a pig-derived nucleic acid, appropriate primers and probes were explored by comparison. In order to explore appropriate primers and probes by comparison, a device into which a pig 12S rRNA nucleotide sequence, which was the target, was dispensed by 1 copy, 2 copies, 4 copies, 8 copies, 16 copies, and 32 copies per well was produced in the same manner as producing a device into which 1 copy was dispensed per well. The respective copy numbers were located on the device at the positions indicated in FIG. 29.

A PCR reagent having the composition described below was added by 16 microliters per well in the produced device.

<PCR Reagent (Composition)>
TaqMan 2×Universal PCR Master Mix*¹: 10 microliters
Forward primer 1 or 1′ *² (10 micromoles): 1 microliter
Reverse primer 2 or 2′*² (10 micromoles): 1 microliter
TaqMan probe*³: 2 microliters

DW: 2 microliters

Total: 16 microliters (per well)

*1: available from Thermo Fisher Scientific Inc.

*2: As regards the primers used, primers indicated by SEQ ID NO. 30, SEQ ID NO. 2, SEQ ID NO. 31, and SEQ ID NO. 3 were synthesized as the primer 1, the primer 1′, the primer 2, and the primer 2′, respectively. As the combinations of the primers, a composition A including 1 and 2 and a composition B including 1′ and 2′ were used.

*3: modified with 5′FAM and 3′TAMRA

As the probe sequence, the nucleotide sequence of SEQ ID NO. 32 was used with the composition A, and the nucleotide sequence of SEQ ID NO. 4 was used with the composition B.

Next, the prepared device was subjected to quantitative PCR reaction and measurement under the conditions described below. The reaction and measurement was performed with QUANT STUDIO 5 of Thermo Fisher Scientific Inc.

<Reaction Conditions>
Pre-heat—
at 50 degrees C. for 2 minutes
at 95 degrees C. for 10 minutes
Cycle—(50 cycles)
at 95 degrees C. for 30 seconds
at 61 degrees C. for 1 minute

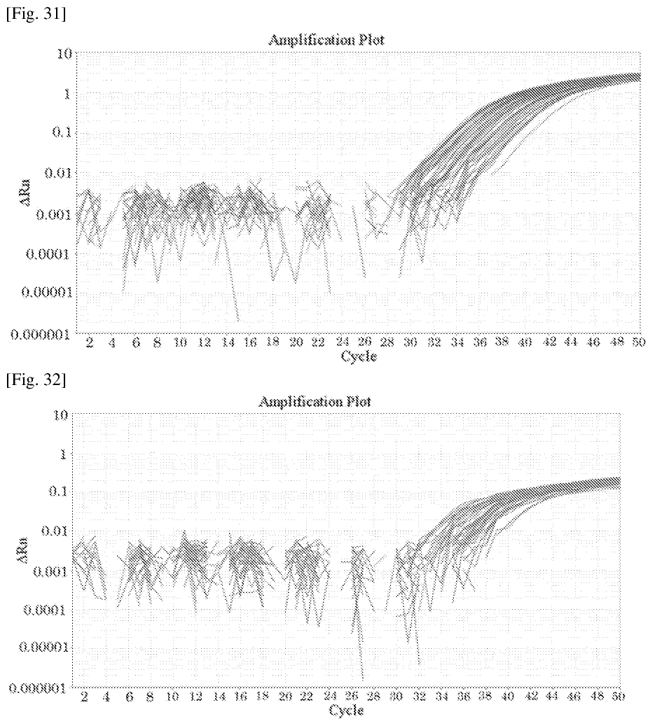

The result was analyzed under Auto setting, with no Baseline Threshold prescribed. The result is plotted in FIG. 30 to FIG. 32.

As plotted in FIG. 30 to FIG. 32, it was revealed that the formulated method was a high-sensitivity qPCR method that was able to detect even 1 copy both with the composition A and the composition B. When differences were extracted, it was confirmed that smaller values were observed with the composition B in the regions of 16 copies and 32 copies, indicating that the reactions were rapid. There is a possibility that rapid reactions would consequently increase the sensitivity, and hence reaction speed may be employed as a criterion for selection. Therefore, the composition B can be evaluated as better than the composition A.

As plotted in FIG. 30 to FIG. 32, as regards fluorescence intensity, the difference between the background value to the Max value was greater with the composition A than with the composition B. This means that the composition A was better in terms of reaction stability.

As can be understood from the foregoing, the present disclosure makes it possible to more clearly know the performance of each testing scheme, and is useful for selecting a testing scheme.

Aspects of the present disclosure are, for example, as follows.

<1> A device including

a well provided in a number of at least one,

wherein a nucleic acid having at least one of a full-length nucleotide sequence and a partial nucleotide sequence of rRNA or rDNA is contained in a defined copy number in at least one well of the well, and

wherein the defined copy number of the nucleic acid having at least one of a full-length nucleotide sequence and a partial nucleotide sequence of rRNA or rDNA is 1,000 or less.

<2> The device according to <1>,

wherein the nucleic acid having at least one of a full-length nucleotide sequence and a partial nucleotide sequence of rRNA or rDNA is contained in a carrier.

<3> The device according to <2>,

wherein the carrier is at least any one selected from the group consisting of cells, phages, and viruses.

<4> The device according to <3>,

wherein the cells are selected from the group consisting of yeast fungi, animal cells, and plant cells.

<5> The device according to any one of <1> to <4>, including

a sealing member configured to seal an opening of the well in which the nucleic acid having at least one of a full-length nucleotide sequence and a partial nucleotide sequence of rRNA or rDNA is contained.

<6> The device according to any one of <1> to <5>,

wherein a number in which the well in which the nucleic acid having at least one of a full-length nucleotide sequence and a partial nucleotide sequence of rRNA or rDNA is contained is present is two or greater, and

wherein the defined copy number of the nucleic acid having at least one of a full-length nucleotide sequence and a partial nucleotide sequence of rRNA or rDNA in one of the well is different from the defined copy number of the nucleic acid having at least one of a full-length nucleotide sequence and a partial nucleotide sequence of rRNA or rDNA in another one of the well.

<7> The device according to any one of <1> to <6>,

wherein the well (1) which is different from the well in which the nucleic acid having at least one of a full-length nucleotide sequence and a partial nucleotide sequence of rRNA or rDNA is contained, and (2) in which a testing target sample is located contains an amplifiable reagent different from the testing target sample.

<8> The device according to any one of <1> to <7>,

wherein the nucleic acid having at least one of a full-length nucleotide sequence and a partial nucleotide sequence of rRNA or rDNA is (1) a nucleic acid having at least one of a full-length nucleotide sequence and a partial nucleotide sequence of pig 12S rRNA or rDNA or (2) a nucleic acid having at least one of a full-length nucleotide sequence and a partial nucleotide sequence of eel 16S rRNA or rDNA.

<9> The device according to any one of <1> to <8>,

wherein the nucleic acid having at least one of a full-length nucleotide sequence and a partial nucleotide sequence of rRNA or rDNA has: (1) at least one of a full-length nucleotide sequence and a partial nucleotide sequence of SEQ ID NO. 1, which is a nucleotide sequence of pig 12S rDNA; or (2) at least one of a full-length nucleotide sequence and a partial nucleotide sequence of SEQ ID NO. 5, which is a nucleotide sequence of eel 16S rDNA, and

wherein a total length of the nucleic acid having at least one of a full-length nucleotide sequence and a partial nucleotide sequence of rRNA or rDNA is 50 nucleotides or more.

<10> The device according to any one of <1> to <9>,

wherein the nucleic acid having at least one of a full-length nucleotide sequence and a partial nucleotide sequence of rRNA or rDNA includes a nucleotide sequence having a homology of 80% or higher with respect to a nucleotide sequence of SEQ ID NO. 1. or with respect to a nucleotide sequence having an arbitrary length, or

wherein the nucleic acid having at least one of a full-length nucleotide sequence and a partial nucleotide sequence of rRNA or rDNA includes a nucleotide sequence having a homology of 80% or higher with respect to a nucleotide sequence of SEQ ID NO. 5 or with respect to a nucleotide sequence having an arbitrary length.

<11> The device according to any one of <8> to <10>,

wherein the nucleic acid having at least one of a full-length nucleotide sequence and a partial nucleotide sequence of pig 12S rRNA or rDNA includes a nucleotide sequence

X including: a nucleotide sequence of SEQ ID NO. 1; and a nucleotide sequence having an arbitrary length less than or equal to 1,000 nucleotides at a 5′ terminal side or a 3′ terminal side, and a nucleotide sequence Y having a homology of 80% or higher with respect to the nucleotide sequence X, or

wherein the nucleic acid having at least one of a full-length nucleotide sequence and a partial nucleotide sequence of eel 16S rRNA or rDNA includes a nucleotide sequence X including: a nucleotide sequence of SEQ ID NO. 5; and a nucleotide sequence having an arbitrary length less than or equal to 1,000 nucleotides at a 5′ terminal side or a 3′ terminal side, and a nucleotide sequence Y having a homology of 80% or higher with respect to the nucleotide sequence X.

<12> The device according to any one of <8> to <11>,

wherein the well in which the nucleic acid having at least one of a full-length nucleotide sequence and a partial nucleotide sequence of pig 12S rRNA or rDNA is contained contains at least any one of primers of SEQ ID NOS. 2 and 3, a probe of SEQ ID NO. 4, and an amplification reagent for a PCR reaction or contains at least any one of primers of SEQ ID NOS. 9, 10, 11, 12, 13, and 14 and an amplification reagent for a LAMP reaction, or

wherein the well in which the nucleic acid having at least one of a full-length nucleotide sequence and a partial nucleotide sequence of eel 16S rRNA or rDNA is contained contains at least any one of primers of SEQ ID NOS. 6 and 7, a probe of SEQ ID NO. 8, and an amplification reagent for a PCR reaction or contains at least any one of primers of SEQ ID NOS. 15, 16, 17, 18, 19, and 20 and an amplification reagent for a LAMP reaction.

<13> The device according to any one of <8> to <12>,

wherein the eel is Japanese eel,

wherein the well in which the nucleic acid having at least one of a full-length nucleotide sequence and a partial nucleotide sequence of the Japanese eel 16S rRNA or rDNA is contained contains at least any one of primers of SEQ ID NOS. 21 and 22, a probe of SEQ ID NO. 23, and an amplification reagent for a PCR reaction or contains at least any one of primers of SEQ ID NOS. 24, 25, 26, 27, 28, and 29 and an amplification reagent for a LAMP reaction.

<14> A nucleic acid testing method including

using the device according to any one of <1> to <13> and subjecting a testing target sample and the nucleic acid, which is contained in the defined copy number and has at least one of a full-length nucleotide sequence and a partial nucleotide sequence of rRNA or rDNA, to amplification reaction to detect rRNA or rDNA contained in the testing target sample.

<15> The nucleic acid testing method according to <14>, including:

determining that a nucleic acid having at least one of a full-length nucleotide sequence and a partial nucleotide sequence of rRNA or rDNA is present in the testing target sample and a detection result is positive when the nucleic acid contained in the defined copy number and having at least one of a full-length nucleotide sequence and a partial nucleotide sequence of rRNA or rDNA and the testing target sample are both amplified; and

determining that a nucleic acid having at least one of a full-length nucleotide sequence and a partial nucleotide sequence of rRNA or rDNA is absent or less than or equal to a limit of detection in the testing target sample and a detection result is negative when the nucleic acid contained in the defined copy number and having at least one of a full-length nucleotide sequence and a partial nucleotide sequence of rRNA or rDNA is amplified and the testing target sample is not amplified.

<16> The nucleic acid testing method according to <14> or <15>, including:

filling the well in which the testing target sample is located, except the well in which the nucleic acid having at least one of a full-length nucleotide sequence and a partial nucleotide sequence of rRNA or rDNA is contained in the defined copy number, with an amplifiable reagent different from the testing target sample, and subjecting the nucleic acid contained in the defined copy number and having at least one of a full-length nucleotide sequence and a partial nucleotide sequence of rRNA or rDNA, the testing target sample, and the amplifiable reagent to amplification reaction;

determining that a nucleic acid having at least one of a full-length nucleotide sequence and a partial nucleotide sequence of rRNA or rDNA is present in the testing target sample and a detection result is positive when all of the nucleic acid contained in the defined copy number and having at least one of a full-length nucleotide sequence and a partial nucleotide sequence of rRNA or rDNA, the testing target sample, and the amplifiable reagent are amplified as a result of the subjecting to amplification reaction; and

determining that a nucleic acid having at least one of a full-length nucleotide sequence and a partial nucleotide sequence of rRNA or rDNA is absent or less than or equal to a limit of detection in the testing target sample and a detection result is negative when the nucleic acid contained in the defined copy number and having at least one of a full-length nucleotide sequence and a partial nucleotide sequence of rRNA or rDNA and the amplifiable reagent are amplified but the testing target sample is not amplified as a result of the subjecting to amplification reaction.

<17> The nucleic acid testing method according to any one of <14> to <16>,

wherein the at least one well in which the nucleic acid having at least one of a full-length nucleotide sequence and a partial nucleotide sequence of rRNA or rDNA is contained in the defined copy number in the device includes one well in which the nucleic acid having at least one of a full-length nucleotide sequence and a partial nucleotide sequence of rRNA or rDNA is contained in a predetermined defined copy number and another one well in which the nucleic acid having at least one of a full-length nucleotide sequence and a partial nucleotide sequence of rRNA or rDNA is contained in a defined copy number different from the defined copy number in the one well,

wherein the nucleic acid testing method includes:

subjecting the nucleic acids having at least one of a full-length nucleotide sequence and a partial nucleotide sequence of rRNA or rDNA contained in the one well and the another one well varied in the defined copy number, and the testing target sample to amplification reaction; and

comparing results of amplification of the nucleic acids having at least one of a full-length nucleotide sequence and a partial nucleotide sequence of rRNA or rDNA contained in respective defined copy numbers with a result of amplification of the testing target sample to determine an amount of rRNA or rDNA contained in the testing target sample.

<18> A nucleic acid testing device used in detection of rRNA or rDNA contained in a testing target sample by subjecting a nucleic acid provided in a defined copy number and having at least one of a full-length nucleotide sequence and a partial nucleotide sequence of rRNA or rDNA and the testing target sample to amplification reaction, the nucleic acid testing device including

a determining unit configured to determine that a nucleic acid having at least one of a full-length nucleotide sequence and a partial nucleotide sequence of rRNA or rDNA is present in the testing target sample and a detection result is positive when the nucleic acid provided in the defined copy number and having at least one of a full-length nucleotide sequence and a partial nucleotide sequence of rRNA or rDNA and the testing target sample are both amplified, and determine that a nucleic acid having at least one of a full-length nucleotide sequence and a partial nucleotide sequence of rRNA or rDNA is absent or less than or equal to a limit of detection in the testing target sample and a detection result is negative when the nucleic acid provided in the defined copy number and having at least one of a full-length nucleotide sequence and a partial nucleotide sequence of rRNA or rDNA is amplified and the testing target sample is not amplified.

<19> A gene testing method targeting rRNA or rDNA, the gene testing method including

managing accuracy of an accuracy management target, using a standard substance, of which absolute number is prescribed by counting the rRNA or rDNA, where the absolute number contains uncertainty.

The device according to any one of <1> to <13>, the nucleic acid testing method according to any one of <14> to <17>, the nucleic acid testing device according to <18>, and the gene testing method according to <19> can solve the various problems in the related art and achieve the object of the present disclosure.

### REFERENCE SIGNS LIST

1: device
2: base material
3: well
4: nucleic acid provided in defined copy number and having at least any one of full-length nucleotide sequence and partial nucleotide sequence of rRNA or rDNA
5: sealing member

1. A device comprising
a well provided in a number of at least one,
wherein a nucleic acid that comprises at least one of a full-length nucleotide sequence and a partial nucleotide sequence of rRNA or rDNA is contained in a defined copy number in at least one well of the well, and
wherein the defined copy number of the nucleic acid that comprises at least one of a full-length nucleotide sequence and a partial nucleotide sequence of rRNA or rDNA is 1,000 or less.

2. The device according to claim 1,
wherein the nucleic acid that comprises at least one of a full-length nucleotide sequence and a partial nucleotide sequence of rRNA or rDNA is contained in a carrier.

3. The device according to claim 2,
wherein the carrier comprises at least any one selected from the group consisting of cells, phages, and viruses.

4. The device according to claim 3,
wherein the cells are selected from the group consisting of yeast fungi, animal cells, and plant cells.

5. The device according to claim 1, comprising
a sealing member configured to seal an opening of the well in which the nucleic acid that comprises at least one of a full-length nucleotide sequence and a partial nucleotide sequence of rRNA or rDNA is contained.

6. The device according to claim 1,
wherein a number in which the well in which the nucleic acid that comprises at least one of a full-length nucleotide sequence and a partial nucleotide sequence of rRNA or rDNA is contained is present is two or greater, and
wherein the defined copy number of the nucleic acid that comprises at least one of a full-length nucleotide sequence and a partial nucleotide sequence of rRNA or rDNA in one of the well is different from the defined copy number of the nucleic acid that comprises at least one of a full-length nucleotide sequence and a partial nucleotide sequence of rRNA or rDNA in another one of the well.

7. The device according to claim 1,
wherein the well (1) which is different from the well in which the nucleic acid that comprises at least one of a full-length nucleotide sequence and a partial nucleotide sequence of rRNA or rDNA is contained, and (2) in which a testing target sample is located contains an amplifiable reagent different from the testing target sample.

8. The device according to claim 1,
wherein the nucleic acid that comprises at least one of a full-length nucleotide sequence and a partial nucleotide sequence of rRNA or rDNA comprises (1) a nucleic acid that comprises at least one of a full-length nucleotide sequence and a partial nucleotide sequence of pig 12S rRNA or rDNA or (2) a nucleic acid that comprises at least one of a full-length nucleotide sequence and a partial nucleotide sequence of eel 16S rRNA or rDNA.

9. The device according to claim 1,
wherein the nucleic acid that comprises at least one of a full-length nucleotide sequence and a partial nucleotide sequence of rRNA or rDNA comprises (1) at least one of a full-length nucleotide sequence and a partial nucleotide sequence of SEQ ID NO. 1, which is a nucleotide sequence of pig 12S rDNA or (2) at least one of a full-length nucleotide sequence and a partial nucleotide sequence of SEQ ID NO. 5, which is a nucleotide sequence of eel 16S rDNA, and
wherein a total length of the nucleic acid that comprises at least one of a full-length nucleotide sequence and a partial nucleotide sequence of rRNA or rDNA is 50 nucleotides or more.

10. The device according to claim 1,
wherein the nucleic acid that comprises at least one of a full-length nucleotide sequence and a partial nucleotide sequence of rRNA or rDNA comprises a nucleotide sequence having a homology of 80% or higher with respect to a nucleotide sequence of SEQ ID NO. 1. or with respect to a nucleotide sequence having an arbitrary length, or
wherein the nucleic acid that comprises at least one of a full-length nucleotide sequence and a partial nucleotide sequence of rRNA or rDNA comprises a nucleotide sequence having a homology of 80% or higher with respect to a nucleotide sequence of SEQ ID NO. 5 or with respect to a nucleotide sequence having an arbitrary length.

11. The device according to claim 8,
wherein the nucleic acid that comprises at least one of a full-length nucleotide sequence and a partial nucleotide sequence of pig 12S rRNA or rDNA comprises a nucleotide sequence X that comprises: a nucleotide sequence of SEQ ID NO. 1; and a nucleotide sequence having an arbitrary length less than or equal to 1,000 nucleotides at a 5′ terminal side or a 3′ terminal side, and a nucleotide sequence Y having a homology of 80% or higher with respect to the nucleotide sequence X, or
wherein the nucleic acid that comprises at least one of a full-length nucleotide sequence and a partial nucleotide sequence of eel 16S rRNA or rDNA comprises a nucleotide sequence X that comprises: a nucleotide sequence of SEQ ID NO. 5; and a nucleotide sequence having an arbitrary length less than or equal to 1,000 nucleotides at a 5′ terminal side or a 3′ terminal side, and a nucleotide sequence Y having a homology of 80% or higher with respect to the nucleotide sequence X.

12. The device according to claim 8,
wherein the well in which the nucleic acid that comprises at least one of a full-length nucleotide sequence and a partial nucleotide sequence of pig 12S rRNA or rDNA is contained contains at least any one of primers of SEQ ID NOS. 2 and 3, a probe of SEQ ID NO. 4, and an amplification reagent for a PCR reaction or contains at least any one of primers of SEQ ID NOS. 9, 10, 11, 12, 13, and 14 and an amplification reagent for a LAMP reaction, or
wherein the well in which the nucleic acid that comprises at least one of a full-length nucleotide sequence and a partial nucleotide sequence of eel 16S rRNA or rDNA is contained contains at least any one of primers of SEQ ID NOS. 6 and 7, a probe of SEQ ID NO. 8, and an amplification reagent for a PCR reaction or contains at least any one of primers of SEQ ID NOS. 15, 16, 17, 18, 19, and 20 and an amplification reagent for a LAMP reaction.

13. The device according to claim 8,
wherein the eel is Japanese eel,
wherein the well in which the nucleic acid that comprises at least one of a full-length nucleotide sequence and a partial nucleotide sequence of the Japanese eel 16S rRNA or rDNA is contained contains at least any one of primers of SEQ ID NOS. 21 and 22, a probe of SEQ ID NO. 23, and an amplification reagent for a PCR reaction or contains at least any one of primers of SEQ ID NOS. 24, 25, 26, 27, 28, and 29 and an amplification reagent for a LAMP reaction.

14. A nucleic acid testing method comprising
the device according to claim 1 and subjecting a testing target sample and the nucleic acid, which is contained in the defined copy number and comprises at least one of a full-length nucleotide sequence and a partial nucleotide sequence of rRNA or rDNA, to amplification reaction to detect rRNA or rDNA contained in the testing target sample.

15. The nucleic acid testing method according to claim 14, comprising:
determining that a nucleic acid that comprises at least one of a full-length nucleotide sequence and a partial nucleotide sequence of rRNA or rDNA is present in the testing target sample and a detection result is positive when the nucleic acid, which is contained in the defined copy number and comprises at least one of a full-length nucleotide sequence and a partial nucleotide sequence of rRNA or rDNA, and the testing target sample are both amplified; and
determining that a nucleic acid that comprises at least one of a full-length nucleotide sequence and a partial nucleotide sequence of rRNA or rDNA is absent or less than a limit of detection in the testing target sample and a detection result is negative when the nucleic acid, which is contained in the defined copy number and comprises at least one of a full-length nucleotide sequence and a partial nucleotide sequence of rRNA or rDNA is amplified and the testing target sample is not amplified.

16. The nucleic acid testing method according to claim 14, comprising:
filling the well in which the testing target sample is located, except the well in which the nucleic acid that comprises at least one of a full-length nucleotide sequence and a partial nucleotide sequence of rRNA or rDNA is contained in the defined copy number, with an amplifiable reagent different from the testing target sample, and subjecting the nucleic acid, which is contained in the defined copy number and comprises at least one of a full-length nucleotide sequence and a partial nucleotide sequence of rRNA or rDNA, the testing target sample, and the amplifiable reagent to amplification reaction;
determining that a nucleic acid that comprises at least one of a full-length nucleotide sequence and a partial nucleotide sequence of rRNA or rDNA is present in the testing target sample and a detection result is positive when all of the nucleic acid, which is contained in the defined copy number and comprises at least one of a full-length nucleotide sequence and a partial nucleotide sequence of rRNA or rDNA, the testing target sample, and the amplifiable reagent are amplified as a result of the subjecting to amplification reaction; and
determining that a nucleic acid that comprises at least one of a full-length nucleotide sequence and a partial nucleotide sequence of rRNA or rDNA is absent or less than a limit of detection in the testing target sample and a detection result is negative when the nucleic acid, which is contained in the defined copy number and comprises at least one of a full-length nucleotide sequence and a partial nucleotide sequence of rRNA or rDNA and the amplifiable reagent are amplified but the testing target sample is not amplified as a result of the subjecting to amplification reaction.

17. The nucleic acid testing method according to claim 14,
wherein the at least one well in which the nucleic acid that comprises at least one of a full-length nucleotide sequence and a partial nucleotide sequence of rRNA or rDNA is contained in the defined copy number in the device comprises one well in which the nucleic acid that comprises at least one of a full-length nucleotide sequence and a partial nucleotide sequence of rRNA or rDNA is contained in a predetermined defined copy number and another one well in which the nucleic acid that comprises at least one of a full-length nucleotide sequence and a partial nucleotide sequence of rRNA or rDNA is contained in a defined copy number different from the defined copy number in the one well,
wherein the nucleic acid testing method comprises:
subjecting the nucleic acids that comprise at least one of a full-length nucleotide sequence and a partial nucleotide sequence of rRNA or rDNA contained in the one well and the another one well varied in the defined copy number, and the testing target sample to amplification reaction; and
comparing results of amplification of the nucleic acids that comprise at least one of a full-length nucleotide sequence and a partial nucleotide sequence of rRNA or rDNA contained in respective defined copy numbers with a result of amplification of the testing target sample to determine an amount of rRNA or rDNA contained in the testing target sample.

18. A nucleic acid testing device used in detection of rRNA or rDNA contained in a testing target sample by subjecting a nucleic acid, which is provided in a defined copy number and comprises at least one of a full-length nucleotide sequence and a partial nucleotide sequence of rRNA or rDNA, and the testing target sample to amplification reaction, the nucleic acid testing device comprising
a determining unit configured to determine that a nucleic acid that comprises at least one of a full-length nucleotide sequence and a partial nucleotide sequence of rRNA or rDNA is present in the testing target sample and a detection result is positive when the nucleic acid, which is provided in the defined copy number and comprises at least one of a full-length nucleotide sequence and a partial nucleotide sequence of rRNA or rDNA, and the testing target sample are both amplified, and determine that a nucleic acid that comprises at least one of a full-length nucleotide sequence and a partial nucleotide sequence of rRNA or rDNA is absent or less than or equal to a limit of detection in the testing target sample and a detection result is negative when the nucleic acid, which is provided in the defined copy number and comprises at least one of a full-length nucleotide sequence and a partial nucleotide sequence of rRNA or rDNA is amplified and the testing target sample is not amplified.

19. A gene testing method targeting rRNA or rDNA, the gene testing method comprising
managing accuracy of an accuracy management target, using a standard substance, of which absolute number is prescribed by counting number of the rRNA or rDNA, the absolute number containing uncertainty.


SEQUENCE LISTING (32 sequences)

SEQ ID NO: 1 - DNA, 960 bp, Artificial Sequence
  pig 12S rDNA
cacaggtttggtcctggcctttctattaattcttaataaaattacacatgcaagtatccgcgccccggtgagaatgccctccagatcttaaagatcaaaaggagcaggtatcaagcacacctataacggtagctcataacgccttgctcaaccacacccccacgggaaacagcagtgataaaaattaagccatgaacgaaagtttgactaagttatattaattagagttggtaaatctcgtgccagccaccgcggtcatacgattaacccaaattaatagatccacggcgtaaagagtgtttaagaaaaaaaatcacaatagagttaaattataactaagctgtaaaaagccctagttaaaataaaataacccacgaaagtgactctaataatcctgacacacgatagctaggacccaaactgggattagataccccactatgcctagccctaaacccaaatagttacataacaaaactattcgccagagtactactcgcaactgcctaaaactcaaaggacttggcggtgcttcacatccacctagaggagcctgttctataatcgataaaccccgatagaccttaccaacccttgccaattcagcctatataccgccatcttcagcaaaccctaaaaaggaacaatagtaagcacaatcatagcacataaaaacgttaggtcaaggtgtagcttatgggttggaaagaaatgggctacattttctacatgagtatatccaccacacgaaagtttttatgaaactaaaaacccaaggaggatttagcagtaaatcgagaatagagtgcttgattgaataaggccatgaagcacgcacacaccgcccgtcaccctcctcaagcatgtagtaataaaaataacctatattcaattacacaaccatgcaagaagagacaagtcgtaacaaggtaagcatactggaaagtgtgcttggattac

SEQ ID NO: 2 - DNA, 30 bp, Artificial Sequence
  primer
gcgtaaagagtgtttaagaaaaaaaatcac

SEQ ID NO: 3 - DNA, 22 bp, Artificial Sequence
  primer
tcctagctatcgtgtgtcagga

SEQ ID NO: 4 - DNA, 22 bp, Artificial Sequence
  probe
aaataacccacgaaagtgactc

SEQ ID NO: 5 - DNA, 1704 bp, Artificial Sequence
  eel 16S rDNA
gcaaaacagctagcctaaccacaataaaacaaatgaccaagcatatataacaaaataaacccaaatataaaataaaacattcttccccctaagtataggtgatagaaaaggacaaaacgcgcaatagaaaaagtaccgcaagggaaagctgaaagagaaatgaaacaatccatataagcaaaaaaaagcagagactaaaactcgtaccttttgcatcatggtttagcaagtaaaaatcaagcaaagagaactttagtttgaaaccccgaaactagacgagctactccggggcagcctaattagggccaacccgtctctgtggcaaaagagtgggaagacccccgagtagaggtgataagcctaccgagcctagttatagctggttgcttaagaaatgaatgttagttcagccttatgtaattctataaccaaaacttattaacaaaaagaacaaaagcaatacataagagttagtcaaagggggtacagcccctttgaaacagaacacaaccttattcaggaggacaaggatcataccataaaaggacaaaattacctcagtgggcccaaaagcagccacctgtaaagaaagcgttaaagctccggtaaatacagaccaataataaagacaatacactccccaccccctaaaaatattaagctatcctatgcacacataggagaaacaatgctaaaatcagtaataagagggcccaagccctctcctagcacatgtgtaagtcagaacggaccgaccactgacaatcaacggacccaaacagagagaaaaagaacaaattacaaaaaacaagaaaaatctatttaataccacaaaccgttaacccaacacaggagtgcctaaaggaaagactaaaaggagaagaaggaactcggcaaacacaaaccccgcctgtttaccaaaaacatcgcctcttgctaacaaaggaagtactagaggtcccgcctgccctgtgaccacaaagtttaacggccgcggtatcctgaccgtgcgaaggtagcgtaatcatttgtcttttaaatagagacctgtatgaatggcataacgagggtttaactgtctccttcccctagtcaatgaaattgatctgcccgtgcagaagcggacataaatacataagacgagaagaccctatggagctttagacaaaagatcaaacatgtaaagaaaccaagttaaccaaaaggaacacaaaggccacgaaacccgacgtaaactgatccaaatgtcttcggttggggcgaccatgggggagaaaaaagcccccacgaggaacagggactaatccctaagccaagagagacatctctaagcaacagaaaatctgaccaaaagtgacccagaatactaattctgatcaacgaaccaagttaccctagggataacagcgcaatcctttcccagagtccatatcgacgaaagggtttacgacctcgatgttggatcaggacatcctaatggtgcagccgctattaagggttcgtttgttcaacgattaatagtcctacgtgatctgagttcagaccggagtaatccaggtcggtttctatctatgaacttacttcttcccagtacgaaaggaccggaatgaagagggccaatactaaaagcaagccctacctctacctgctgaaagcaaataaaacagacaaagaggtatattaaacctaccaaagataatggta

SEQ ID NO: 6 - DNA, 23 bp, Artificial Sequence
  primer
ctgatcaacgaaccaagttaccc

SEQ ID NO: 7 - DNA, 22 bp, Artificial Sequence
  primer
gatgtcctgatccaacatcgag

SEQ ID NO: 8 - DNA, 22 bp, Artificial Sequence
  probe
ctttcccagagtccatatcgac

SEQ ID NO: 9 - DNA, 19 bp, Artificial Sequence
  primer
cgttaggtcaaggtgtagc

SEQ ID NO: 10 - DNA, 20 bp, Artificial Sequence
  primer
tccagtatgcttaccttgtt

SEQ ID NO: 11 - DNA, 45 bp, Artificial Sequence
  primer
tcaatcaagcactctattctcgctacatgagtatatccaccacac

SEQ ID NO: 12 - DNA, 37 bp, Artificial Sequence
  primer
gccatgaagcacgcacacacgacttgtctcttcttgc

SEQ ID NO: 13 - DNA, 21 bp, Artificial Sequence
  primer
ctgctaaatcctccttgggtt

SEQ ID NO: 14 - DNA, 21 bp, Artificial Sequence
  primer
cctcctcaagcatgtagtaat

SEQ ID NO: 15 - DNA, 43 bp, Artificial Sequence
  primer
tcgtcgatatggactctgggaaaatcctgatcaacgaaccaag

SEQ ID NO: 16 - DNA, 43 bp, Artificial Sequence
  primer
cgacctcgatgttggatcagggaacaaacgaacccttaatagc

SEQ ID NO: 17 - DNA, 20 bp, Artificial Sequence
  primer
ggattgcgctgttatcccta

SEQ ID NO: 18 - DNA, 19 bp, Artificial Sequence
  primer
acatcctaatggtgcagcc

SEQ ID NO: 19 - DNA, 21 bp, Artificial Sequence
  primer
taagccaagagatacacctct

SEQ ID NO: 20 - DNA, 21 bp, Artificial Sequence
  primer
tcagatcacgtaggactgtta

SEQ ID NO: 21 - DNA, 26 bp, Artificial Sequence
  primer
gatcaaacatgtaaagaaaccaagtt

SEQ ID NO: 22 - DNA, 24 bp, Artificial Sequence
  primer
atcagaattagtattctgggtcac

SEQ ID NO: 23 - DNA, 24 bp, Artificial Sequence
  probe
gaggaacagggactaatccctaag

SEQ ID NO: 24 - DNA, 47 bp, Artificial Sequence
  primer
cgccccaaccgaagacatttggatcaaacatgtaaagaaaccaagtt

SEQ ID NO: 25 - DNA, 47 bp, Artificial Sequence
  primer
aacagggactaatccctaagccaagaattagtattctgggtcacttt

SEQ ID NO: 26 - DNA, 21 bp, Artificial Sequence
  primer
gatcagtttacgtcgggtttc

SEQ ID NO: 27 - DNA, 24 bp, Artificial Sequence
  primer
gagagacatctctaagcaacagaa

SEQ ID NO: 28 - DNA, 19 bp, Artificial Sequence
  primer
gacgagaagaccctatgga

SEQ ID NO: 29 - DNA, 20 bp, Artificial Sequence
  primer
caatcctttcccagagtcca

SEQ ID NO: 30 - DNA, 20 bp, Artificial Sequence
  primer
ctatgcctagccctaaaccc

SEQ ID NO: 31 - DNA, 20 bp, Artificial Sequence
  primer
taggtggatgtgaagcaccg

SEQ ID NO: 32 - DNA, 26 bp, Artificial Sequence
  probe
attcgccagagtactactcgcaactg